(19) United States
(12) Patent Application Publication
(10) Pub. No.: US 2021/0247468 A1
(43) Pub. Date: Aug. 12, 2021

(54) SYSTEMS AND METHODS THAT EXPLOIT MAXWELL'S EQUATIONS AND GEOMETRY TO REDUCE NOISE FOR ULTRA-FINE MEASUREMENTS OF MAGNETIC FIELDS FROM THE BRAIN USING A NEURAL DETECTION SYSTEM

(71) Applicant: HI LLC, Los Angeles, CA (US)

(72) Inventors: Benjamin Shapiro, Culver City, CA (US); Zachary Bednarke, Los Angeles, CA (US); Ricardo Jiménez-Martínez, Culver City, CA (US); Julian Kates-Harbeck, Marina Del Rey, CA (US)

(73) Assignee: HI LLC, Los Angeles, CA (US)

(21) Appl. No.: 17/160,179

(22) Filed: Jan. 27, 2021

Related U.S. Application Data
(60) Provisional application No. 62/975,723, filed on Feb. 12, 2020.
(60) Provisional application No. 63/035,683, filed on Jun. 5, 2020.

(51) Int. Cl. G01R 33/02; G01R 33/565; G01R 33/56; A61B 5/245

(52) U.S. Cl. CPC G01R 33/0206; A61B 5/245; G01R 33/5608; G01R 33/56581

(57) ABSTRACT

Measurements of an arbitrary magnetic field having one or more magnetic field components are acquired from a plurality of magnetometers, and a generic model of at least one of the one or more magnetic field components of the arbitrary magnetic field is generated in the vicinity of the magnetometers. The generic magnetic field model comprises an initial number of different basis functions. Maxwell's equations are applied to the generic magnetic field model to reduce the initial number of different basis functions, thereby yielding a Maxwell-constrained model of the magnetic field component(s) of the arbitrary magnetic field, and the magnetic field component(s) of the arbitrary magnetic field are estimated at each of at least one of the magnetometers based on the constrained magnetic field model and the arbitrary magnetic field measurements acquired from each magnetometer.

### RELATED APPLICATION DATA

Pursuant to 35 U.S.C. § 119(e), this application claims the benefit of U.S. Provisional Patent Application 62/975,723, filed Feb. 12, 2020, and U.S. Provisional Patent Application 63/035,683, filed Jun. 5, 2020, which are expressly incorporated herein by reference.

### FIELD OF THE INVENTION

The present inventions relate to methods and systems for non-invasive measurements from the human body, and in particular, methods and systems related to detecting physiological activity from the human brain, animal brain, and/or peripheral nerves.

### BACKGROUND OF THE INVENTION

Measuring neural activity in the brain is useful for medical diagnostics, neuromodulation therapies, neuroengineering, and brain-computer interfacing. Conventional methods for measuring neural activity in the brain include X-Ray Computed Tomography (CT) scans, positron emission tomography (PET), functional magnetic resonance imaging (fMRI), or other methods that are large, expensive, require dedicated rooms in hospitals and clinics, and are not wearable or convenient to use.

In contrast to these techniques, one promising technique for measuring neural activity in the brain is magnetoencephalography (MEG), which is capable of non-invasively detecting neural activity in the brain without potentially harmful ionizing radiation, and without use of heavy or large equipment. Thus, MEG-based neural activity measurement systems can be scaled to wearable or portable form factors, which is especially important in brain-computer interface (BCI) applications that require subjects to interact freely within their environment. MEG operates under the principle that time-varying electrical current within activated neurons inherently generate magnetic signals in the form of a magnetic field that can be detected by very sensitive magnetometers located around the head.

Measuring the small magnetic fields emanating from the brain, and doing so non-invasively (without surgically penetrating the skin and bone of the head) and doing so with high spatial and temporal resolution, is difficult. The magnetic fields produced by the brain are small, and they are smaller still by the time they propagate out past the skull and the skin surface of the head. In comparison, the magnetic field emitted from various outside magnetic sources in the environment, including from global sources, such as the Earth's magnetic field, and from localized sources, such as electrical outlets and sockets, electrical wires or connections in the wall, and everyday electrical equipment in a home, office, or laboratory setting, far exceed the strength of the magnetic signals generated in the brain by many orders of magnitude, and has a distribution in space and time that is not known a-priori. Hence, it is a difficult challenge to extract the small desired signal from the brain, and to discriminate it from much larger unwanted magnetic field signals from the rest of the user's natural environment.

One type of system that can be used for MEG is a Superconductive Quantum Interference Device (SQUID), which is sensitive enough to measure magnetic fields as small as 5×10⁻¹⁸ Tesla, which can be compared to magnetic fields resulting from physiological processes in animals, which may be in the range of 10⁻⁹ to 10⁻⁶ Tesla. However, SQUIDs rely on superconducting loops, and thus require cryogenic cooling, which may make it prohibitively costly and too large to be incorporated into a wearable or portable form factor. Thus, neural activity measurement systems that utilize SQUIDs may not be appropriate for BCI applications.

Optically pumped magnetometers (OPMs) have emerged as a viable and wearable alternative to cryogenic, superconducting, SQUID-based MEG systems, and have an advantage of obviating the need for cryogenic cooling, and as a result, may be flexibly placed on any part of the body, including around the head, which is especially important for BCI applications. Because cryogenic cooling is not required, OPMs may be placed within millimeters of the scalp, thereby enabling measurement of a larger signal from the brain (brain signals dissipate with distance), especially for sources of magnetic signals at shallow depths beneath the skull, as well as providing consistency across different head shapes and sizes.

OPMs optically pump a sample (usually a vapor formed of one of the alkali metals (e.g., rubidium, cesium, or potassium) due to their simple atomic structure, low melting point, and ease of pumping with readily available lasers) with circularly polarized light at a precisely defined frequency, thereby transferring polarized light to the vapor, and producing a large macroscopic polarization in the vapor in the direction of the light (i.e., the alkali metal atoms in the vapor will all have spins that are oriented in the direction of the light) that induces a magnetically sensitive state in the vapor. Once this magnetically sensitive state is established, polarized light is no longer transferred to the vapor, but instead, passes transparently through the vapor. In the presence of an ambient magnetic field, the spin orientation (or precession) of the alkali metal atoms in the optically pumped vapor will uniformly change, thereby disrupting the magnetically sensitive state, which is then subsequently reestablished by the transfer of the polarized light to the vapor. Because the transmission of light through the vapor varies as the spin precession of the alkali metal atoms in the vapor (and thus the magnetically sensitive state) changes in response to changes in the ambient magnetic field, the transmission of light (either the pumping light or a separate probe light) through the vapor represents a magnetic field-dependent signal (i.e., a MEG signal) that may be detected, thereby providing a measure of magnitude changes in the magnetic field.

To maintain the magnetically sensitive state of the vapor, it is important that spin relaxation due to spin exchange collisions be suppressed. In low magnetic fields (<10 nT), spin relaxation due to spin exchange collisions can be suppressed greatly, and thus, some OPMs are operated as zero-field magnetometers or Spin Exchange Relaxation Free (SERF) OPMs (referred to as “SERF OPMs”), thereby allowing for very high magnetometer sensitivities. Furthermore, because OPM measurements can be quite sensitive to low-frequency noise, the polarization of the vapor may be modulated to move the MEG signal away from the low-frequency end of the spectrum. SERF OPMs typically amplitude modulate the vapor polarization using magnetic coils that generate oscillating magnetic fields that vary at a frequency (e.g., 2000 Hz) much greater than the relaxation rate of the vapor (approximately 100 Hz). The amplitude modulated MEG signal can then be demodulated using lock-in detection to recover the MEG signal.

Although SERF OPMs allow for very high magnetometer sensitivities, they have a small dynamic range and bandwidth compared to SQUIDs, and can thus only operate in small magnetic fields (tens of nT, and often lower, to stay in the linear range of the OPMs). This becomes problematic when attempting to detect a very weak neural activity-induced magnetic field from the brain against an outside magnetic field.

For example, referring to FIG. 1, the magnitude of the magnetic field generated by a human brain (i.e., the MEG signal) may range from below 5 fT to just below 1 pT, while the magnitude of the outside magnetic field, including the Earth's magnetic field, may range from just above 5 μT to 100 μT. It should be appreciated that Earth's magnetic field covers a large range as it depends on the position of the Earth, as well as the materials of the surrounding environment where the magnetic field is measured. There are also magnetic fields from electrical power lines, everyday electric objects (microwaves, fridges, cell phones), and their interaction with magnetizable objects (metal chair legs, tables, metal posts, wall rebar, etc.). In the United States these magnetic fields appear at 60 Hz and its harmonics (120 Hz, 180 Hz, etc.) and can range in amplitude from about 500 nT to below 10 nT. In Europe electrical power is at 50 Hz, with harmonics at 100 Hz, 150 Hz, etc., and similar magnitudes.

The approximate operating range of a SERF OPM (i.e., the range in which the metallic alkali vapor resonates) extends from below 1 fT up to 200 nT. Outside of this range, the metallic alkali vapor in the OPM loses sensitivity to magnetic fields. In contrast, the approximate operating range of a less sensitive sensor, such as a flux gate magnetometer, extends from around 100 fT to close to 100 μT. Thus, in contrast to flux gate magnetometers, the limited dynamic range of a SERF OPM presents a challenge in measuring signals having a high dynamic range, e.g., approximately 2×10¹⁰, which corresponds to the ratio of the lower range magnitude of the MEG signal (approximately 5 fT) to the higher range magnitude of the outside magnetic field (approximately 100 μT).

Thus, to take advantage of SERF OPMs for MEG, the outside magnetic field must be suppressed to near-zero. Otherwise, the SERF OPM cannot operate. One conventional technique for suppressing the outside magnetic field involves using large, immobile, and expensive magnetically shielded rooms to passively isolate the SERF OPMs from the sources of the outside magnetic field, effectively reducing the dynamic range requirements of the SERF OPMs used to measure the weak MEG signals.

These shielded rooms, however, are generally not viable for the consumer market, especially with regard to BCI applications, where it desirable that the MEG-based neural activity measurement system be incorporated into a wearable or portable form factor. Thus, for BCI applications, SERF OPMs must be capable of operating in the ambient background magnetic field of the native environment, including the Earth's magnetic field and other local sources of magnetic fields.

Another technique for suppressing the outside magnetic field without using magnetically shielded rooms involves incorporating a direct broadband feedback control system to actively null the outside magnetic field at the SERF OPM. In this case, the system actuators attempt to cancel the entire bandwidth of the outside magnetic field by applying a noise-cancelling, broadband, magnetic field to the sensors. However, such feedback control for OPM systems has not been implemented in a wearable system.

There, thus, remains a need to provide means for more effectively suppressing an outside magnetic field in a wearable neural detection system.

### SUMMARY OF THE INVENTION

In accordance with a first aspect of the present inventions, a system comprises a plurality of magnetometers (e.g., a plurality of coarse magnetometers, such as flux gate magnetometers, and a plurality of fine magnetometers, such as optically pumped magnetometers (OPMs)) configured for taking measurements of an arbitrary magnetic field having one or more magnetic field components. The system further comprises a processor configured for acquiring the arbitrary magnetic field measurements from the plurality of magnetometers, and generating a generic model of at least one of the one or more magnetic field components of the arbitrary magnetic field in the vicinity of the plurality of magnetometers. The generic magnetic field model comprises an initial number of different basis functions (e.g., 0^{th} order basis functions and 1st order basis functions or at least one non-linear basis function, such as, e.g., a vector spherical harmonics (VSH) basis function).

The processor is further configured for applying Maxwell's equations to the generic magnetic field model to reduce the initial number of different basis functions, thereby yielding a Maxwell-constrained model of the magnetic field component(s) of the arbitrary magnetic field, estimating the magnetic field component(s) of the arbitrary magnetic field at each of at least one of the plurality of magnetometers (e.g., fine magnetometers) based on the constrained magnetic field model and the arbitrary magnetic field measurements acquired from each of the magnetometer(s).

In one embodiment, the magnetic field component(s) of the magnetic field measurement acquired from each of the magnetometer(s) comprises a physical portion and a non-physical portion, and the magnetic field component estimate(s) at the magnetometer(s) has a physical portion and a non-physical portion. The non-physical portion of the magnetic field component estimate(s) at each of the magnetometer(s) is respectively less than the non-physical portion of the magnetic field component of the magnetic field measurement acquired from each of the magnetometer(s).

In another embodiment, the processor is configured for estimating the magnetic field component(s) at each of the magnetometer(s) by parameterizing the constrained magnetic field model at least partially based on the arbitrary magnetic field measurements acquired from the plurality of magnetometers, thereby yielding a parameterized model of magnetic field component(s) of the arbitrary magnetic field in the vicinity of the plurality of magnetometers, and substituting each location of the magnetometer(s) into the parameterized magnetic field model. In this embodiment, the processor may be configured for parameterizing the constrained magnetic field model by fitting the coefficients of the reduced number of basis functions of the constrained magnetic field model at least partially to the arbitrary magnetic field measurements acquired from the plurality of magnetometers (e.g., using a least squares optimization technique), and incorporating the fitted coefficients into the constrained magnetic field model.

In still another embodiment, the magnetic field component(s) of the arbitrary magnetic field of which the measurements are taken comprises an outside magnetic field and a magnetoencephalography (MEG) magnetic field, the magnetic field component(s) of the arbitrary magnetic field of which the generic model is generated comprises the outside magnetic field, the initial number of different basis functions in the generic magnetic field model comprises basis functions for the outside magnetic field, and the magnetic field component estimate(s) at each of the magnetometer(s)(s) comprises an outside magnetic field estimate.

In this embodiment, the magnetic field component(s) of the arbitrary magnetic field of which the generic model is generated may further comprise the MEG magnetic field, the initial number of different basis functions in the generic magnetic field model may further comprise basis functions for the MEG magnetic field, and the magnetic field component estimate(s) at each of the magnetometer(s) may further comprise a MEG magnetic field estimate.

In this embodiment, the arbitrary magnetic field may be a total residual magnetic field, and the system may further comprise at least one magnetic field actuator (e.g., three orthogonal magnetic field actuators, each of which may be uniform) configured for generating an actuated magnetic field that at least partially cancels the outside magnetic field at each of the magnetometer(s), thereby yielding the total residual magnetic field at each of the magnetometer(s), such that the arbitrary magnetic field measurements acquired from the plurality of magnetometers are total residual magnetic field measurements acquired from the plurality of magnetometers. In this case, the processor is configured for estimating the total residual magnetic field at each of the magnetometer(s) based on the outside magnetic field estimate at each of the magnetometer(s) and the total residual magnetic field measurements acquired from the plurality of magnetometers, and controlling the actuated magnetic field at least partially based on the total residual magnetic field estimate at each of the magnetometer(s) in a manner that suppresses the total residual magnetic field at each of the magnetometer(s) to a baseline level, such that each at each of the magnetometer(s) is in-range.

In this embodiment, the processor may be configured for estimating the total residual magnetic field at each of the magnetometer(s) by determining a known actuated magnetic field at each of the magnetometer(s) (e.g., by summing the known actuated magnetic field at each of the magnetometer(s) and the outside magnetic field estimate at each of the magnetometer(s)), and estimating the total residual magnetic field at each of the magnetometer(s) based on the known actuated magnetic field at each of the magnetometer(s) and the outside magnetic field estimate at each of the magnetometer(s). Each of the magnetic field actuator(s) may respectively have at least one actuation strength, in which case, the processor may be configured for determining the known actuated magnetic field at each of the magnetometer(s) based on a known profile of the magnetic field actuator(s) and the actuation strength(s) of the magnetic field actuator(s).

In yet another embodiment, the system further comprises a signal acquisition unit configured for being worn on a head of a user. The signal acquisition unit comprises a support structure, the magnetic field actuator(s) affixed to the support structure, and the plurality of magnetometers affixed to the support structure. The signal acquisition unit is configured for deriving a MEG signal from the total residual magnetic field estimate at each of the magnetometer(s). The system further comprises a signal processing unit configured for determining an existence of neural activity in the brain of the user at least partially based on the MEG signal derived from the total residual magnetic field estimate at each of the magnetometer(s).

In accordance with a second aspect of the present inventions, a method comprises acquiring measurements (e.g., coarse total residual magnetic field measurements and fine total residual magnetic field measurements) of an arbitrary magnetic field having one or more magnetic field components at a plurality of detection locations. The method further comprises generating a generic model of at least one of the magnetic field component(s) of the arbitrary magnetic field in the vicinity of the plurality of detection locations. The generic magnetic field model comprises an initial number of different basis functions (e.g., 0^{th} order basis functions and 1st order basis functions or at least one non-linear basis function, such as, e.g., a vector spherical harmonics (VSH) basis function). The method further comprises applying Maxwell's equations to the generic magnetic field model to reduce the initial number of different basis functions, thereby yielding a Maxwell-constrained model of the magnetic field component(s) of the arbitrary magnetic field, estimating the at least one magnetic field component of the arbitrary magnetic field at each of at least one of the plurality of detection locations (e.g., fine detection locations) based on the constrained magnetic field model and the arbitrary magnetic field measurements acquired from each of the detection location(s).

In one method, the magnetic field component(s) of the magnetic field measurement acquired from each of the detection location(s) comprises a physical portion and a non-physical portion, and the magnetic field component estimate(s) at the detection location(s) has a physical portion and a non-physical portion. The non-physical portion of magnetic field component estimate(s) at each of the detection location(s) is respectively less than the non-physical portion of the magnetic field component(s) of the magnetic field measurement acquired from each of the detection location(s).

In another method, estimating the magnetic field component(s) at each of the detection location(s) comprises parameterizing the constrained magnetic field model at least partially based on the arbitrary magnetic field measurements acquired from the plurality of detection locations, thereby yielding a parameterized model of the magnetic field component(s) of the arbitrary magnetic field in the vicinity of the plurality of detection locations, and substituting each of the detection location(s) into the parameterized magnetic field model. In this method, parameterizing the constrained magnetic field model may comprise fitting the coefficients of the reduced number of basis functions of the constrained magnetic field model at least partially to the arbitrary magnetic field measurements acquired from the plurality of detection locations (e.g., using a least squares optimization technique), and incorporating the fitted coefficients into the constrained magnetic field model.

In still another method, the magnetic field component(s) of the arbitrary magnetic field of which the measurements are taken comprises an outside magnetic field and a magnetoencephalography (MEG) magnetic field, the magnetic field component(s) of the arbitrary magnetic field of which the generic model is generated comprises the outside magnetic field, the initial number of different basis functions in the generic magnetic field model comprises basis functions for the outside magnetic field, and the magnetic field component estimate(s) at each of the detection location(s) comprises an outside magnetic field estimate.

In this method, the magnetic field component(s) of the arbitrary magnetic field of which the generic model is generated may further comprise the MEG magnetic field, the initial number of different basis functions in the generic magnetic field model may further comprise basis functions for the MEG magnetic field, and the magnetic field component estimate(s) at each of the detection location(s) may further comprise a MEG magnetic field estimate.

In this method, the arbitrary magnetic field may be a total residual magnetic field, and the method may further comprise generating an actuated magnetic field (e.g., a uniform actuated magnetic field generated in three dimensions) that at least partially cancels the outside magnetic field at each of the magnetometer(s), thereby yielding the total residual magnetic field at each of the detection location(s), such that the arbitrary magnetic field measurements acquired from the plurality of detection locations are total residual magnetic field measurements acquired from the plurality of detection locations. In this case, the total residual magnetic field is estimated at each of the detection location(s) based on the outside magnetic field estimate at each of the detection location(s) and the total residual magnetic field measurements acquired from the plurality of detection locations, and controlling the actuated magnetic field at least partially based on the total residual magnetic field estimate at each of the detection location(s) in a manner that suppresses the total residual magnetic field at each of the detection location(s) to a baseline level, such that an accuracy of the total residual magnetic field at each of the detection location(s) increases.

In this method, total residual magnetic field at each of the detection location(s) may be estimated by determining a known actuated magnetic field at each of the detection location(s) (e.g., by summing the known actuated magnetic field at each of the detection location(s) and the outside magnetic field estimate at each of the detection location(s)), and estimating the total residual magnetic field at each of the detection location(s) based on the known actuated magnetic field at each of the detection location(s) and the outside magnetic field estimate at each of the detection location(s). The known actuated magnetic field at each of detection location may be determined based on a known profile of actuated magnetic field and an actuation strength of the actuated magnetic field.

Yet another method further comprises deriving a MEG signal from the total residual magnetic field estimate at each of the detection location(s), and determining an existence of neural activity in the brain of a user at least partially based on the MEG signal derived from the total residual magnetic field estimate at each of the detection location(s).

In accordance with a third aspect of the present inventions, a system comprises a plurality of magnetometers configured for taking measurements of a magnetic field containing a magnetoencephalography (MEG) magnetic field emanating from a brain of a user, such that the magnetic field measurement taken at each of at least one of the plurality of magnetometers has a MEG magnetic field component. The MEG magnetic field component of the magnetic field measurement taken at each of the magnetometer(s) has a physical portion and a non-physical portion.

The system further comprises a processor configured for acquiring the magnetic field measurements from the plurality of magnetometers, and suppressing the non-physical portion of the MEG magnetic field component of the magnetic field measurement acquired from each of the magnetometer(s) relative to the physical portion of the MEG magnetic field component of the magnetic field measurement acquired from each of the magnetometer(s).

In one embodiment, the magnetic field further comprises an outside magnetic field, such that the magnetic field measurement acquired from each of the magnetometer(s) further has an outside magnetic field component. In this case, the processor is configured for suppressing the outside magnetic field component of the magnetic field measurement acquired from each of the magnetometer(s) relative to the MEG magnetic field component of the magnetic field measurement acquired from each of the magnetometer(s).

In one specific implementation of this embodiment, the processor may be configured for suppressing the outside magnetic field measurement component of the magnetic field measurement acquired from each of the magnetometer(s) relative to the MEG magnetic field component of the magnetic field measurement acquired from each of the magnetometer(s) based on one or more of a temporal frequency of the outside magnetic field (e.g., by suppressing the magnetic field measurement acquired from the each at least one magnetometer at DC and harmonic temporal frequencies), a spatial frequency of the outside magnetic field (e.g., by suppressing the magnetic field measurement acquired from the each at least one magnetometer at relatively low spatial frequencies), and a strength of the outside magnetic field (e.g., by suppressing the magnetic field measurement acquired from each of the magnetometer(s) at relatively high strength frequency components).

In another embodiment, the processor is configured for suppressing the outside magnetic field measurement component of the magnetic field measurement acquired from each of the magnetometer(s) relative to the MEG magnetic field component of the magnetic field measurement acquired from each of the magnetometer(s) by generating a generic model of the MEG magnetic field in the vicinity of the plurality of magnetometers. The generic MEG magnetic field model comprises an initial number of different basis functions. The generic magnetic field model comprises an initial number of different basis functions (e.g., 0^{th} order basis functions and 1st order basis functions or at least one non-linear basis function, such as, e.g., a vector spherical harmonics (VSH) basis function). The processor is further configured for applying Maxwell's equations to the generic MEG magnetic field model to reduce the initial number of different basis functions, thereby yielding a Maxwell-constrained model of the MEG magnetic field model, and estimating the MEG magnetic field model at each magnetometer based on the constrained MEG magnetic field model and the magnetic field measurements acquired from the plurality of magnetometers.

In this embodiment, the processor is configured for estimating the MEG magnetic field model at each of the magnetometer(s) by parameterizing the constrained MEG magnetic field model at least partially based on the magnetic field measurements acquired from the plurality of magnetometers, thereby yielding a parameterized model of the outside magnetic field in the vicinity of the plurality of magnetometers, and substituting a location of each of the magnetometer(s) into the parameterized magnetic field model. In this embodiment, the processor may be configured for parameterizing the constrained magnetic field model by fitting the coefficients of the reduced number of basis functions of the constrained magnetic field model at least partially to the arbitrary magnetic field measurements acquired from the plurality of magnetometers (e.g., using a least squares optimization technique), and incorporating the fitted coefficients into the constrained magnetic field model, e.g., the Maxwell-constrained outside magnetic field model.

In yet another embodiment, the system further comprises a signal acquisition unit configured for being worn on a head of a user. The signal acquisition unit comprises a support structure, the magnetic field actuator(s) affixed to the support structure, and the plurality of magnetometers affixed to the support structure. The signal acquisition unit is configured for deriving a MEG signal from the magnetic field estimate at each of the magnetometer(s). The system further comprises a signal processing unit configured for determining an existence of neural activity in the brain of the user at least partially based on the MEG signal derived from the magnetic field measurement at each of the magnetometer(s).

In accordance with a fourth aspect of the present inventions, a method comprises acquiring measurements of an arbitrary magnetic field respectively at a plurality of detection locations. The arbitrary magnetic field comprises a magnetoencephalography (MEG) magnetic field emanating from a brain of a user, such that the magnetic field measurement taken at each of at least one of the plurality of detection locations has a MEG magnetic field component. The MEG magnetic field component of the magnetic field measurement acquired from each detection location has a physical portion and a non-physical portion. The method further comprises suppressing the non-physical portion of the MEG magnetic field component of the magnetic field measurement acquired from each detection location relative to the physical portion of the MEG magnetic field component of the magnetic field measurement acquired from each detection location.

In one method, the magnetic field further comprises an outside magnetic field, such that the magnetic field measurement acquired from each detection location further has an outside magnetic field component, in which case, the processor is configured for suppressing the outside magnetic field component of the magnetic field measurement acquired from each detection location relative to the MEG magnetic field component of the magnetic field measurement acquired from each detection location. The outside magnetic field measurement component of the magnetic field measurement acquired from each detection location may be suppressed relative to the MEG magnetic field component of the magnetic field measurement acquired from each detection location based on one or more of a temporal frequency of the outside magnetic field (e.g., by suppressing the magnetic field measurement acquired from the each at least one magnetometer at DC and harmonic temporal frequencies), a spatial frequency of the outside magnetic field (e.g., by suppressing the magnetic field measurement acquired from the each at least one magnetometer at relatively low spatial frequencies), and a strength of the outside magnetic field (e.g., by suppressing the magnetic field measurement acquired from each of the magnetometer(s) at relatively high strength frequency components).

In another method, suppressing the outside magnetic field measurement component of the magnetic field measurement acquired from each one detection location relative to the MEG magnetic field component of the magnetic field measurement acquired from each detection location comprises generating a generic model of the MEG magnetic field in the vicinity of the plurality of detection locations.

The generic magnetic field model comprises an initial number of different basis functions (e.g., 0^{th} order basis functions and 1st order basis functions or at least one non-linear basis function, such as, e.g., a vector spherical harmonics (VSH) basis function). The method further comprises applying Maxwell's equations to the generic magnetic field model to reduce the initial number of different basis functions, thereby yielding a Maxwell-constrained model of the MEG magnetic field model, and estimating the MEG magnetic field model at each detection location based on the constrained MEG magnetic field model and the magnetic field measurements acquired from the plurality of detection locations.

In this method, estimating the MEG magnetic field model at the each of at least one detection location comprises parameterizing the constrained MEG magnetic field model at least partially based on the magnetic field measurements acquired from the plurality of detection locations, thereby yielding a parameterized model of the outside magnetic field in the vicinity of the plurality of detection locations, and substituting each detection location into the parameterized magnetic field model. Parameterizing the constrained MEG magnetic field model may comprise fitting the coefficients of the reduced number of basis functions of the constrained magnetic field model at least partially to the magnetic field measurements acquired from the plurality of detection locations (e.g., using a least squares optimization technique), and incorporating the fitted coefficients into the Maxwell-constrained outside magnetic field model.

Yet another method comprises deriving a MEG signal from the magnetic field measurement at each detection location, and determining an existence of neural activity in the brain of the user at least partially based on the MEG signal derived from the magnetic field measurement acquired from each detection location.

In accordance with a fifth aspect of the present inventions, a system comprises a plurality of magnetometers (e.g., a plurality of coarse magnetometers, such as flux gate magnetometers, and a plurality of fine magnetometers, such as optically pumped magnetometers (OPMs)) configured for taking measurements of an arbitrary magnetic field having a plurality of magnetic field components. The system further comprises a processor configured for acquiring the arbitrary magnetic field measurements from the plurality of magnetometers, and generating a generic model of the plurality of magnetic field components of the arbitrary magnetic field in the vicinity of the plurality of magnetometers. The generic magnetic field model comprises a plurality of basis functions having multiple sets of basis functions respectively corresponding to the plurality of magnetic field components of the arbitrary magnetic field, and the processor is further configured for parameterizing the generic magnetic field model by simultaneously fitting coefficients of the plurality of basis functions at least partially to the arbitrary magnetic field measurements acquired from the plurality of magnetometers (e.g., using a least squares optimization technique), thereby yielding a parameterized model of the plurality of magnetic field components of the arbitrary magnetic field in the vicinity of the plurality of magnetometers.

In one embodiment, the plurality of magnetic field components of the arbitrary magnetic field comprises a physical portion of an outside magnetic field and a non-physical portion of the outside magnetic field, the first set of basis functions correspond to modes of the outside magnetic field that are physically possible, and the second set of basis functions correspond to modes of the outside magnetic field that are physically impossible. In another embodiment, the plurality of magnetic field components of the arbitrary magnetic field comprises a magnetoencephalography (MEG) magnetic field and an outside magnetic field, the first set of basis functions correspond to modes in the MEG magnetic field, and the second set of basis functions correspond to modes in the outside magnetic field. In still another embodiment, the plurality of magnetic field components of the arbitrary magnetic field comprises a magnetoencephalography (MEG) magnetic field of interest and a magnetoencephalography (MEG) magnetic field not of interest, the first set of basis functions correspond to modes of the MEG magnetic field of interest, and the second set of basis functions correspond to modes of the MEG magnetic field not of interest.

The processor is further configured for estimating a first one of the plurality of magnetic field components of the arbitrary magnetic field at each of at least one of the plurality of magnetometers (e.g., fine magnetometers) based on a first one of the multiple sets of basis functions of the parameterized magnetic field model. The processor may be configured for estimating the first one of the plurality of magnetic field components of the arbitrary magnetic field at each of the magnetometer(s) based on the parameterized magnetic field model by substituting a location of each of the magnetometer(s) into the parameterized magnetic field model.

In one embodiment, the processor is further configured for estimating a second one of the plurality of magnetic field components of the arbitrary magnetic field at each of the magnetometer(s) based on a second one of the multiple sets of basis functions of the parameterized magnetic field model.

In another embodiment, the generic magnetic field model comprises a coefficient vector and a matrix of influence from the coefficient vector to the plurality of magnetic field components of the arbitrary magnetic field. The coefficient vector has a p number of coefficients respectively corresponding to the plurality of basis functions. The influence matrix comprises a p number of column vectors and an N number of row vectors respectively corresponding to the arbitrary magnetic field measurements acquired from the plurality of magnetometers, where p is less than N. The processor is configured for simultaneously fitting the coefficients of the plurality of basis functions at least partially to the arbitrary magnetic field measurements acquired from the plurality of magnetometers by equating the product of the coefficient vector and the influence matrix to the arbitrary magnetic field measurements acquired from the plurality of magnetometers, and simultaneously fitting the p number of coefficients in the coefficient vector at least partially to the arbitrary magnetic field measurements acquired from the plurality of magnetometers.

In still another embodiment, the plurality of magnetic field components of the arbitrary magnetic field comprises an outside magnetic field, and the estimated first one of the plurality of magnetic field components at each of the magnetometer(s) is an outside magnetic field estimate at each of the magnetometer(s). In this embodiment, the system further comprises at least one magnetic field actuator (e.g., three orthogonal magnetic field actuators, each of which may be uniform) configured for generating an actuated magnetic field that at least partially cancels the outside magnetic field at each of the magnetometer(s), thereby yielding a total residual magnetic field at each of the magnetometer(s) as the arbitrary magnetic field. In this embodiment, the processor is configured for estimating the total residual magnetic field at each of the magnetometer(s) based on the outside magnetic field estimate at each of the magnetometer(s) and the total residual magnetic field measurements acquired from the plurality of magnetometers.

In one specific implementation of this embodiment, the processor may be configured for estimating the total residual magnetic field at each of the magnetometer(s) by determining a known actuated magnetic field at each of the magnetometer(s) (e.g., by summing the known actuated magnetic field at each of the magnetometer(s) and the outside magnetic field estimate at each of the magnetometer(s)), and estimating the total residual magnetic field at each of the magnetometer(s) based on the known actuated magnetic field at each of the magnetometer(s) and the outside magnetic field estimate at each of the magnetometer(s). Each of the magnetic field actuator(s) may respectively have at least one actuation strength, in which case, the processor may be configured for determining the known actuated magnetic field at each of the magnetometer(s) based on a known profile of the magnetic field actuator(s) and the actuation strength(s) of the magnetic field actuator(s).

In this embodiment, the processor is further configured for controlling the actuated magnetic field at least partially based on the total residual magnetic field estimate at each of the magnetometer(s) in a manner that suppresses the total residual magnetic field at each of the magnetometer(s) to a baseline level, such that each of the magnetometer(s) is in-range.

In this embodiment, the system may further comprise a signal acquisition unit configured for being worn on a head of a user. The signal acquisition unit comprises a support structure, the magnetic field actuator(s) affixed to the support structure, and the plurality of magnetometers affixed to the support structure. The signal acquisition unit is configured for deriving at least one MEG signal(s) from the total residual magnetic field estimate at each of the magnetometer(s). The system further comprises a signal processing unit configured for determining an existence of neural activity in the brain of the user at least partially based on the MEG signal(s) derived from the total residual magnetic field estimate at each of the magnetometer(s).

In accordance with a sixth aspect of the present inventions, a method comprises acquiring measurements (e.g., coarse total residual magnetic field measurements and fine total residual magnetic field measurements) of an arbitrary magnetic field having a plurality of magnetic field components respectively from a plurality of detection locations. The method further comprises generating a generic model of the plurality of magnetic field components of the arbitrary magnetic field in the vicinity of the plurality of detection locations. The generic magnetic field model comprises an initial plurality of basis functions having multiple sets of basis functions.

The method further comprises parameterizing the generic magnetic field model by simultaneously fitting coefficients of the plurality of basis functions at least partially to the arbitrary magnetic field measurements acquired from the plurality of detection locations (e.g., using a least squares optimization technique), thereby yielding a parameterized model of the plurality of magnetic field components of the arbitrary magnetic field in the vicinity of the plurality of detection locations.

In one method, the plurality of magnetic field components of the arbitrary magnetic field comprises a physical portion of an outside magnetic field and a non-physical portion of the outside magnetic field, the first set of basis functions correspond to modes of the outside magnetic field that are physically possible, and the second set of basis functions correspond to modes of the outside magnetic field that are physically impossible. In another method, the plurality of magnetic field components of the arbitrary magnetic field comprises a magnetoencephalography (MEG) magnetic field and an outside magnetic field, the first set of basis functions correspond to modes in the MEG magnetic field, and the second set of basis functions correspond to modes in the outside magnetic field. In still another method, the plurality of magnetic field components of the arbitrary magnetic field comprises a magnetoencephalography (MEG) magnetic field of interest and a magnetoencephalography (MEG) magnetic field not of interest, the first set of basis functions correspond to modes of the MEG magnetic field of interest, and the second set of basis functions correspond to modes of the MEG magnetic field not of interest.

The method further comprises estimating a first one of the plurality of magnetic field components of the arbitrary magnetic field at each of at least one of the plurality of detection locations (e.g., fine detection locations) based on a first one of the multiple sets of basis functions. Estimating the first one of the plurality of magnetic field components of the arbitrary magnetic field at each of the detection location(s) based on the parameterized magnetic field model may comprise substituting each detection location(s) into the parameterized magnetic field model.

One method further comprises estimating a second one of the plurality of magnetic field components of the arbitrary magnetic field at each of the detection location(s) based on a second one of the multiple sets of basis functions.

In another method, the generic magnetic field model comprises a coefficient vector and a matrix of influence from the coefficient vector to the plurality of magnetic field components of the arbitrary magnetic field, the coefficient vector having a p number of coefficients respectively corresponding to the plurality of basis functions. The influence matrix comprises a p number of column vectors and an N number of row vectors respectively corresponding to the arbitrary magnetic field measurements acquired from the plurality of detection locations, where p is less than N. The coefficients of the plurality of basis functions may be simultaneously fitted at least partially to the arbitrary magnetic field measurements acquired from the plurality of detection locations by equating the product of the coefficient vector and the influence matrix to the arbitrary magnetic field measurements acquired from the plurality of detection locations and simultaneously fitting the p number of coefficients in the coefficient vector at least partially to the arbitrary magnetic field measurements acquired from the plurality of detection locations.

In still another method, the magnetic field component(s) of the arbitrary magnetic field comprises an outside magnetic field, and the estimated first one of the plurality of magnetic field components at each of the detection location(s) is an outside magnetic field estimate at each of the detection location(s). This method further comprises generating an actuated magnetic field (e.g., a uniform actuated magnetic field generated in three dimensions) that at least partially cancels an outside magnetic field at each of the detection location(s), thereby yielding a total residual magnetic field as the arbitrary magnetic field at each of the detection location(s). This method further comprises estimating the total residual magnetic field at each of the detection location(s) based on the outside magnetic field estimate at each of the detection location(s) and the total residual magnetic field measurements acquired from the plurality of detection locations (e.g., by summing the known actuated magnetic field at each of the detection location(s) and the outside magnetic field estimate at each of the detection location(s)). In this method, the known actuated magnetic field may be determined at each of the detection location(s) based on a known profile of the actuated magnetic field an actuation strength of the actuated magnetic field.

The method further comprises controlling the actuated magnetic field at least partially based on the total residual magnetic field estimate at each of the detection location(s) in a manner that suppresses the total residual magnetic field at each of the detection location(s) to a baseline level, such that an accuracy of the total residual magnetic field measurement acquired from each of the detection location(s) increases.

This method may further comprise deriving a MEG signal from the total residual magnetic field estimate at each of the detection location(s), and determining an existence of neural activity in the brain of a user at least partially based on the MEG signal derived from the total residual magnetic field estimate at each of the detection location(s).

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings.

Understanding that these drawings depict only typical embodiments of the present inventions and are not therefore to be considered limiting of its scope, the present inventions will be described and explained with additional specificity and detail through the use of the accompanying drawings in which:

FIG. 1 is a diagram of illustrating dynamic ranges of a magnetoencephalography (MEG) signal and a typical outside magnetic field, and the operating ranges of a Spin Exchange Relaxation Free (SERF) optically-pumped magnetometer (OPM) and flux gate magnetometer, plotted on a magnetic spectrum;

FIG. 2 is a block diagram of a neural activity measurement system constructed in accordance with one embodiment of the present inventions, particularly shown in the context of a brain computer interface (BCI);

FIG. 3 is a side view of a physical implementation of the BCI of FIG. 3;

FIG. 4 is a block diagram of one exemplary embodiment of a signal acquisition unit used by the neural activity measurement system of FIG. 2;

FIG. 5 is a diagram illustrating three different magnetic field distinguishing techniques employed by the signal acquisition unit of FIG. 4;

FIG. 6 is a diagram illustrating strengths, temporal frequencies, and spatial frequencies of a typical outside magnetic field, typical magnetoencephalography (MEG) magnetic field, and measurement noise;

FIG. 7 is a diagram illustrating a total residual magnetic field measured by the signal acquisition unit of FIG. 4, particularly showing exemplary frequency components of an outside magnetic field, MEG magnetic field, and measurement noise;

FIG. 8 is a diagram illustrating the spatial frequency of a typical outside magnetic field, typical magnetoencephalography (MEG) magnetic field, and measurement noise relative to magnetometers of the signal acquisition unit of FIG. 4;

FIG. 9 is a flow diagram illustrating one exemplary generic method of operating the signal acquisition unit of FIG. 4;

FIG. 10 is a flow diagram illustrating one exemplary method of estimating an environmental magnetic field of total residual magnetic field measurements by the signal acquisition unit of FIG. 4; and

FIG. 11 is a flow diagram illustrating another exemplary method of estimating one or more magnetic field components of total residual magnetic field measurements by the signal acquisition unit of FIG. 4.

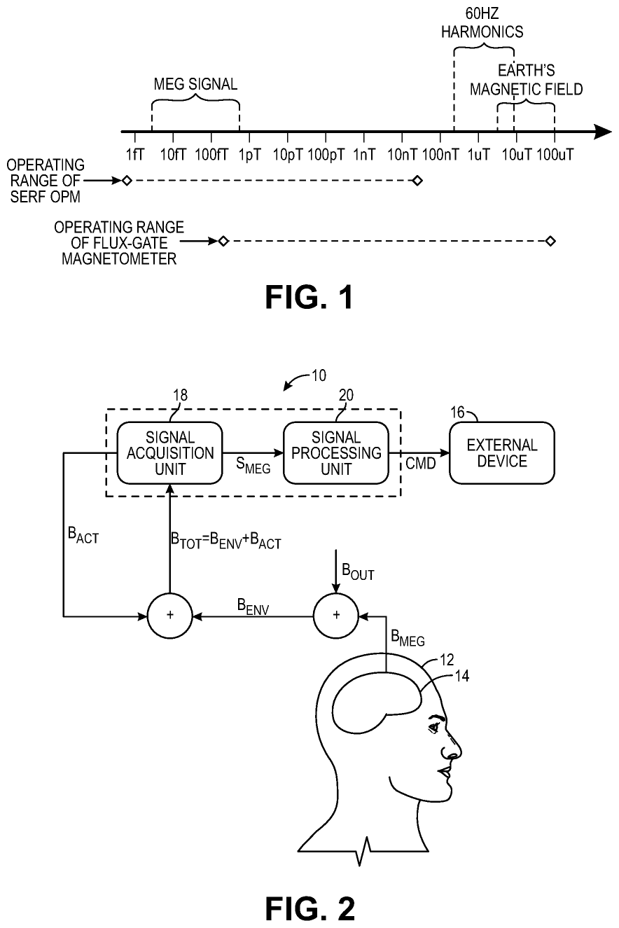

### DETAILED DESCRIPTION OF THE EMBODIMENTS

Significantly, the neural activity measurement systems (and variations thereof) described herein are configured for non-invasively acquiring magnetoencephalography (MEG) signals from a brain of a user while effectively cancelling an outside magnetic field without the use of magnetically shielded rooms, and identifying and localizing the neural activity within the cortical structures of the brain of the user based on the acquired magnetoencephalography (MEG) signals.

The neural activity measurement system described herein may take the form of a brain computer interface (BCI) (also known as a neural-controlled interface (NCI), mind-machine interface (MMI), direct neural interface (DNI), or brain-machine interface (BMI)), which converts the neural activity information into commands that are output to an external device or devices for carrying out desired actions that replace, restore, enhance, supplement, or improve natural central nervous system (CNS) output, and thereby changes the ongoing interactions between the CNS of a user and an external or internal environment.

For example, as illustrated in FIG. 2, one embodiment of a neural activity measurement system 10 constructed in accordance with the present inventions will be described. The neural activity measurement system 10 is configured for measuring neural activity in the brain 14 of a user 12, generating commands CMD in response to the measured neural activity information, and sending the commands CMD to an external device 16 in the context of a BCI.

To this end, the neural activity measurement system 10 generally comprises a signal acquisition unit 18 configured for at least partially cancelling a relatively strong outside magnetic field B_{OUT} within an environmental magnetic field B_{ENV} that also includes a relatively weak MEG magnetic field B_{MEG} induced by electrical current (indicative of neural activity) in a brain 14 of a user 12. That is, B_{TOT}=B_{ENV}+B_{ACT}=B_{OUT}+B_{MEG}+B_{ACT}. The outside magnetic field B_{OUT} may emanate from global sources (e.g., the Earth's magnetic field), and from localized sources, including, but not limited to, from electromagnetic radiation emanating from electrical outlets and sockets, electrical wires or connections in the wall, and everyday electrical equipment (microwave ovens, televisions, refrigerators, environmental systems (air conditioning, etc.) in a home, office, or laboratory setting, as well as from cell phones, biomagnetics unrelated to neural signals (such as facial muscles, magnetic fields produced by the heart or nerves firing), everyday objects encountered inside (metal and magnetic objects, including steel supports, rebar, studs, utility boxes, etc.) and outside spaces, such as cell phone towers, power lines, transformers, and moving vehicles (e.g., cars, trains, bikes, electric bikes and scooters, electric cars, etc.), user motion/rotation/translation in a background field (earth field), user clothing and eyeglasses, personal electronics (e.g., laptop computers, watches, phones, smart rings, etc.), active implantable medical devices (pacemakers), augmented reality/virtual reality, sound systems (that use magnets), etc.

The signal acquisition unit 18 is configured for generating an actuated magnetic field B_{ACT} that at least partially cancels the relative strong outside magnetic field B_{OUT} within the environmental magnetic field B_{ENV}, yielding a total residual magnetic field B_{TOT} (which is preferably zero or near-zero due to the summation of the environmental magnetic field B_{ENV} and the actuated magnetic field B_{ACT}). The signal acquisition unit 18 is further configured for detecting the total residual magnetic field B_{TOT} as feedback to cancel the outside magnetic field B_{OUT}. The signal acquisition unit 18 is also configured for extracting and outputting a clean (i.e., reduced-noise) electrical MEG signals S_{MEG} of the MEG magnetic field B_{MEG} from the total residual magnetic field B_{TOT}.

The signal acquisition unit 18 may utilize any suitable technique for acquiring the MEG magnetic field B_{MEG}, including, but not limited to the techniques described in U.S. patent application Ser. No. 16,428,871, entitled “Magnetic Field Measurement Systems and Methods of Making and Using,” U.S. patent application Ser. No. 16/418,478, entitled “Magnetic Field Measurement System and Method of Using Variable Dynamic Range Optical Magnetometers”, U.S. patent application Ser. No. 16/418,500, entitled, “Integrated Gas Cell and Optical Components for Atomic Magnetometry and Methods for Making and Using,” U.S. patent application Ser. No. 16/457,655, entitled “Magnetic Field Shaping Components for Magnetic Field Measurement Systems and Methods for Making and Using,” U.S. patent application Ser. No. 16/213,980, entitled “Systems and Methods Including Multi-Basis function Operation of Optically Pumped Magnetometer(s),” (now U.S. Pat. No. 10,627,460), U.S. patent application Ser. No. 16/456,975, entitled “Dynamic Magnetic Shielding and Beamforming Using Ferrofluid for Compact Magnetoencephalography (MEG),” U.S. patent application Ser. No. 16/752,393, entitled “Neural Feedback Loop Filters for Enhanced Dynamic Range Magnetoencephalography (MEG) Systems and Methods,” U.S. patent application Ser. No. 16/741,593, entitled “Magnetic Field Measurement System with Amplitude-Selective Magnetic Shield,” U.S. Provisional Application Ser. No. 62/858,636, entitled “Integrated Magnetometer Arrays for Magnetoencephalography (MEG) Detection Systems and Methods,” U.S. Provisional Application Ser. No. 62/836,421, entitled “Systems and Methods for Suppression of Non-Neural Interferences in Magnetoencephalography (MEG) Measurements,” U.S. Provisional Application Ser. No. 62/842,818 entitled “Active Shield Arrays for Magnetoencephalography (MEG),” U.S. Provisional Application Ser. No. 62/926,032 entitled “Systems and Methods for Multiplexed or Interleaved Operation of Magnetometers,” U.S. Provisional Application Ser. No. 62/896,929 entitled “Systems and Methods having an Optical Magnetometer Array with Beam Splitters,” and U.S. Provisional Application Ser. No. 62/960,548 entitled “Methods and Systems for Fast Field Zeroing for Magnetoencephalography (MEG),” which are all expressly incorporated herein by reference.

The neural activity measurement system 10 further comprises a signal processing unit 20 configured for processing the electrical MEG signal S_{MEG} to identify and localize neural activity within the cortex of the brain 14 of the user 12, and issuing the commands CMD to the external device 16 in response to the identified and localized neural activity in the brain 14 of the user 12.

It should be appreciated that, although the neural activity measurement system 10 is described herein in the context of a BCI, the present inventions should not be so limited, and may be applied to any system used for any application (including, but not limited to, medical, entertainment, neuromodulation stimulation, lie detection devices, alarm, educational, etc.), where it is desirable to perform measurements on a magnetic field induced by any physiological process in a person that would benefit from cancelling the outside magnetic field B_{OUT}. For example, instead of deriving neural activity information from MEG signals, magnetic fields induced by electrical heart activity can be measured to determine heart activity information of a person.

Furthermore, it should also be appreciated that, although the use of the signal acquisition unit lends itself well to neural activity measurement systems, the signal acquisition unit 18 may find use in other applications, such as, e.g., other types of biomedical sensing, vehicle navigation, mineral exploration, non-destructive testing, detection of underground devices, asteroid mining, space exploration, etc. Thus, signal acquisition unit 18 can be adapted to measure neural signals generated from non-brain anatomical structures, as well as other types of biological signals and non-biological signals.

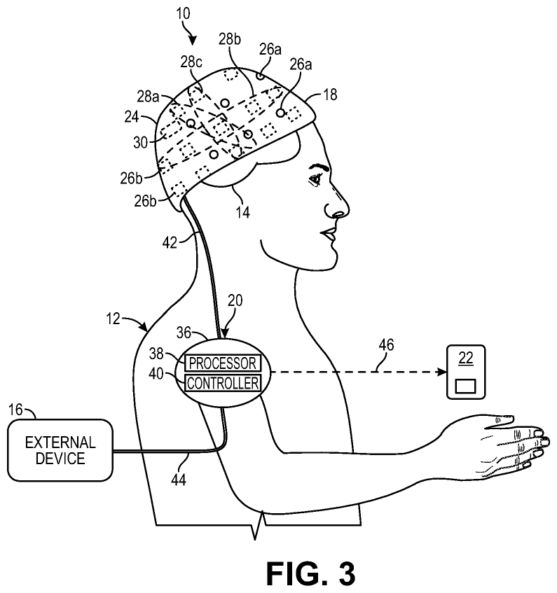

Referring now to FIG. 3, an exemplary physical implementation of the neural activity measurement system 10 will be described.

As shown, the signal acquisition unit 18 is configured for being applied to the user 12, and in this case, worn on the head of the user 12. The signal acquisition unit 18 comprises a support structure 24, a plurality of magnetometers 26 (divided between a plurality of coarse magnetometers 26a and a plurality of fine magnetometers 26b) distributed about the support structure 24, a set of magnetic field actuators 28 in proximity to the fine magnetometers 26b, and a processor 30 electrically coupled between the magnetometers 26 and the set of actuators 28.

The support structure 24 may be shaped, e.g., have a banana, headband, cap, helmet, beanie, other hat shape, or other shape adjustable and conformable to the user's head, such that at least some of the magnetometers 26 are in close proximity, preferably in contact, with the outer skin of the head, and in this case, the scalp of the user 12. The support structure 24 may be made out of any suitable cloth, soft polymer, plastic, hard shell, and/or any other suitable material as may serve a particular implementation. An adhesive, strap, or belt (not shown) can be used to secure the support structure 24 to the head of the user 12.

Each of the magnetometers 26 is configured for detecting a spatial component of the total residual magnetic field B_{TOT}, and outputting a corresponding electrical signal representative of the spatial component of the total residual magnetic field B_{TOT}. In the illustrated embodiment, the plurality of coarse magnetometers 26a is distributed on the outside of the support structure 24 for detecting the respective spatial components of the total residual magnetic field B_{TOT} mainly from outside of the support structure 24, whereas the plurality of fine magnetometers 26b is distributed on the inside of the support structure 24 for detecting the respective spatial components of the total residual magnetic field B_{TOT} mainly from inside the support structure 24 (i.e. they are closer to the brain 14 of the user 12).

Each of the coarse magnetometers 26a has a relatively low sensitivity, but high dynamic sensitivity range, to magnetic fields, whereas each of the fine magnetometers 26b has a relatively high sensitivity, but low dynamic sensitivity range. The signal acquisition unit 18 may have any suitable number of magnetometers 26. For example, the signal acquisition unit 18 may have twelve coarse magnetometers 26a and twenty-five fine magnetometers 26b, although one of ordinary skill in the art would understand that signal acquisition unit 18 may have any suitable number of coarse magnetometers 26a and magnetometers 26b, including more coarse magnetometers 26a then fine magnetometers 26b. In alternative embodiments of the signal acquisition unit 18, the plurality of magnetometers 26 may only comprise a plurality of fine magnetometers 26b distributed on the inside of the support structure 24.

In the illustrated embodiment, each coarse magnetometer 26a takes the form of a flux gate magnetometer, which has a relatively low sensitivity (e.g., on the order of 100 fT), and thus, may not be capable of measuring weak magnetic fields generated by neural activity in the brain 14 of the user 12. However, a flux gate magnetometer has a relatively high dynamic sensitivity range (in the range of 100 fT to close to 100 μT), and thus, may operate in a large outside magnetic field B_{OUT}. Although each of the coarse magnetometers 26a are described as taking the form of a flux gate magnetometer, other types of coarse magnetometers can be used, including, but not limited to, anisotropic magnetoresistance (AMR) sensors, tunnel magnetoresistance (TMR) sensors, Hall-effect sensors, nitrogen vacancy sensors, or any other magnetometer that can operate in a linear range over the amplitude range of a typical outside magnetic field B_{OUT}. As will be described in further detail below, each of the coarse magnetometers 26a is specifically designed to facilitate the calibration of its offset and gain using novel pre-calibration and dynamic calibration techniques.

In the illustrated embodiment, each fine magnetometer 26b takes the form of a Spin Exchange Relaxation Free (SERF) Optically Pumped Magnetometer (OPM). Although a SERF OPM has a relatively small dynamic range (e.g., in the range of 1 ft to 200 nT), it has a relatively high sensitivity (on the order of 1 fT) to magnetic fields compared to flux gate magnetometers. Further details of SERF OPMs are described in U.S. Provisional Application Ser. No. 62/975,693, entitled “Nested and Parallel Feedback Control Loops For Ultra-Fine Measurements of Magnetic Fields From the Brain Using a Wearable MEG System” (Attorney Docket No. KERN-079), which is expressly incorporated herein by reference.

In the illustrated embodiment, each of the coarse magnetometers 26a and fine magnetometers 26b are vector magnetometers that are capable of detecting the total residual magnetic field B_{TOT} in three dimensions (x, y, and z). For example, each of the coarse magnetometers 26a may include a triad of the scalar magnetometers, as described in U.S. Provisional Application Ser. No. 62/975,709, entitled “Self-Calibration of Flux Gate Offset and Gain Drift To Improve Measurement Accuracy Of Magnetic Fields From the Brain Using a Wearable MEG System” (Attorney Docket No. KERN-078), and each of the fine magnetometer 26b may be vector magnetometers, as described in U.S. patent application Ser. No. 16/752,393, entitled “Neural Feedback Loop Filters for Enhanced Dynamic Range Magnetoencephalography (MEG) Systems and Methods,” which are expressly incorporated herein by reference.

The clean (i.e., reduced-noise) electrical MEG signals S_{MEG} that are representative of the spatial components of the MEG magnetic field B_{MEG}, and that will be processed by the signal processing unit 20 for determining and localizing neural activity in the brain 14 of the user 12, will be respectively derived from the electrical signals output by the respective fine magnetometers 26b, and in some cases, from the electrical signals output by the coarse magnetometers 26a; whereas the characteristics (namely amplitude and phase) of the actuated magnetic field B_{ACT} will be derived from the electrical signals output by the respective coarse magnetometers 26a and/or the electrical signals output by at least some of the respective fine magnetometers 26b.

The set of magnetic field actuators 28 is configured for generating the actuated magnetic field B_{ACT} to at least partially cancel the outside magnetic field B_{OUT} in the vicinity of the plurality of fine magnetometers 26b. The set of magnetic field actuators 28 may, e.g., comprise at least one coil and at least one driver that drives the coil(s) with electrical current at a defined amperage, voltage, or some other variable, and at a defined frequency, thereby setting the actuation strengths of the magnetic field actuators 28. In the illustrated embodiment, the set of magnetic field actuators 28 comprises a triad of uniform magnetic field actuators 28a-28c for respectively generating x-, y-, and z-components of the actuated magnetic field B_{ACT} to cancel the outside magnetic field B_{OUT} in all three dimensions. In an optional embodiment, the set of magnetic field actuators 28 may also comprise six gradient magnetic field actuators (not shown) for generating first-order x-, y-, and z-gradient components of the actuated magnetic field B_{ACT}. One of ordinary skill in the art would appreciate that the set of field actuators 28 may include any suitable and type of magnetic field actuators capable of cancelling the outside magnetic field B_{OUT} at the magnetometers 26.

The processor 30 is electrically coupled between the magnetometers 26 and magnetic field actuators 28 via electrical wires (not shown), and is configured for processing the electrical signals respectively output by the coarse magnetometers 26a (and in some cases the electrical signals output by the fine magnetometers 26b) in response to the detection of the spatial components of the total residual magnetic field B_{TOT}, determining the characteristics of the actuated magnetic field B_{ACT} required to cancel the outside magnetic field B_{OUT} in the total residual magnetic field B_{TOT}, and generating cancellation control signals based on this determination that are output to the set of magnetic field actuators 28. Further details discussing novel techniques for cancelling the outside magnetic field B_{OUT} in the total residual magnetic field B_{TOT} are described in U.S. Provisional application Ser. No. 62/xxx,xxx, entitled “Nested and Parallel Feedback Control Loops For Ultra-Fine Measurements of Magnetic Fields From the Brain Using a Wearable MEG System” (Attorney Docket No. KERN-079).

To minimize the size, weight, and cost of the signal acquisition unit 18, the functions of the processor 30 are preferably performed digitally (e.g., in firmware, such as a programmable logic device (e.g., a field programmable gate array (FPGA), or an ASIC (application specific integrated circuit) device, or in a micro-processor)), in which case, one or more analog-to-digital converters (not shown) can be employed between the magnetometers 26 and the processor 30, and one or more digital-to-analog converters (not shown) can be employed between the magnetic field actuators 28 and the processor 30. However, it should be appreciated that, in alternative embodiments, the functions of the processor 30 may be at least partially performed in an analog fashion.

It should be noted that, although the signal acquisition unit 18 is illustrated in FIG. 3 as having a single set of magnetic field actuators 28 and a single processor 30, the signal acquisition unit 18 may comprise more than one set of magnetic field actuators 28 and more than one processor 30. In this case, each set of magnetic field actuators 28 and each corresponding processor 30 may be associated with a subset of magnetometers 26. In one embodiment, the fine magnetometers 26b, set(s) of magnetic field actuators 28, and processor(s) 30 may be fabricated as integrated module(s). For example, each integrated module may comprise a rectangular substrate containing a subset or all of the fine magnetometers 26b, a set of the magnetic field actuators 28 incorporated into the rectangular substrate, such that coils of the magnetic field actuators 28 respectively wrap around the orthogonal dimensions of the rectangular substrate, and the processor 30 affixed to the surface of the rectangular substrate between the coils.

The signal processing unit 20 is configured for being applied to the user 12, and in this case, worn remotely from the head of the user 12, e.g., worn on the neck, shoulders, chest, or arm) of the user 12. The signal processing unit 20 comprises a housing 36 containing a processor 38 and a controller 40. The processor 38 is configured for identifying and localizing neural activity within the cortex of the brain 14 of the user 12, and the controller 40 is configured for issuing commands CMD to an external device 16 in response to the identified and localized neural activity in the brain 14 of the user 12, as well as controlling the high-level operational functions of the signal acquisition unit 18. The signal processing unit 20 may additionally include a power supply (which if head-worn, may take the form of a rechargeable or non-chargeable battery), a control panel with input/output functions, a display, and memory. Alternatively, power may be provided to the signal processing unit 20 wirelessly (e.g., by induction).

In the illustrated embodiment, the neural activity measurement system 10 further comprises a wired connection 42 (e.g., electrical wires) for providing power from the signal processing unit 20 to the signal acquisition unit 18 and communicating between the signal processing unit 20 and the signal acquisition unit 18. Alternatively, the neural activity measurement system 10 may use a non-wired connection (e.g., wireless radio frequency (RF) signals (e.g., Bluetooth, Wifi, cellular, etc.) or optical links (e.g., fiber optic or infrared (IR)) for providing power from the signal processing unit 20 to the signal acquisition unit 18 and/or communicating between the signal processing unit 20 and the signal acquisition unit 18.

In the illustrated embodiment, the neural activity measurement system 10 further comprises a wired connection 44 (e.g., electrical wires) for providing power from the signal processing unit 20 to the external device 16 and communicating between the signal processing unit 20 and the external device 16. Alternatively, the neural activity measurement system 10 may use a non-wired connection (e.g., wireless radio frequency (RF) signals (e.g., Bluetooth, Wifi, cellular, etc.) or optical links (e.g., fiber optic or infrared (IR)) for providing power from the signal processing unit 20 to the external device 16 and/or communicating between the signal processing unit 20 and the external device 16.

The neural activity measurement system 10 may optionally comprise a remote processor 22 (e.g., a Smartphone, tablet computer, or the like) in communication with the signal processing unit 20 coupled via a wired connection (e.g., electrical wires) or a non-wired connection (e.g., wireless radio frequency (RF) signals (e.g., Bluetooth, Wifi, cellular, etc.) or optical links (e.g., fiber optic or infrared (IR)) 46. The remote processor 22 may store data from previous sessions, and include a display screen.

It should be appreciated that at least a portion of the signal acquisition and magnetic field cancellation functionality of the processor 30 in the signal acquisition unit 18 may be implemented in the signal processing unit 20, and/or at least a portion of the neural activity determination and localization functionality of the signal processing unit 20 may be implemented in the signal acquisition unit 18. In the preferred embodiment, the functionalities of the processor 30 in the signal acquisition unit 18, as well as the processor 38 and a controller 40 in the signal processing unit 20, may be implemented using one or more suitable computing devices or digital processors, including, but not limited to, a microcontroller, microprocessor, digital signal processor, graphical processing unit, central processing unit, application specific integrated circuit (ASIC), field programmable gate array (FPGA), and/or programmable logic unit (PLU). Such computing device(s) or digital processors may be associated with non-transitory computer- or processor-readable medium that stores executable logic or instructions and/or data or information, which when executed, perform the functions of these components. The non-transitory computer- or processor-readable medium may be formed as one or more registers, for example of a microprocessor, FPGA, or ASIC, or can be a type of computer-readable media, namely computer-readable storage media, which may include, but is not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The signal acquisition unit 18 takes advantage of the high dynamic range of the coarse magnetometers 26a to compensate for the relatively low dynamic range of the fine magnetometers 26b to cancel the large outside magnetic field B_{OUT}, while also taking advantage of high sensitivity of the fine magnetometers 26b to compensate for the low sensitivity of the coarse magnetometers 26a to measure the MEG signal S_{MEG}.

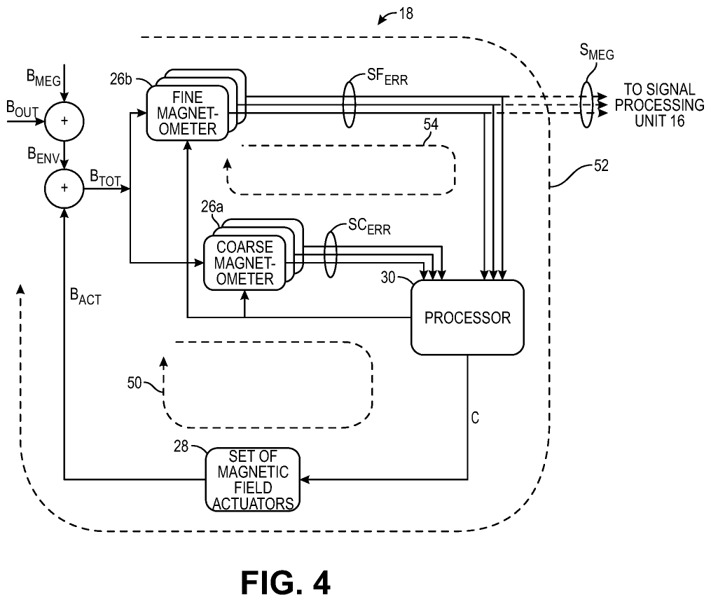

In particular, and with reference to FIG. 4, the signal acquisition unit 18 is configured for at least partially cancelling the outside magnetic field B_{OUT} at the locations of the fine magnetometers 26b by initially employing a coarse feedback control loop 50 having a relatively low sensitivity, but relatively high dynamic range, for coarsely cancelling the outside magnetic field B_{OUT} (e.g., low-frequency cancellation of the outside magnetic field B_{OUT} contributed by the Earth's magnetic field (e.g., any of the techniques described in U.S. patent application Ser. No. 16/752,393, entitled “Neural Feedback Loop Filters for Enhanced Dynamic Range Magnetoencephalography (MEG) Systems and Methods,” which is expressly incorporated herein by reference, a broadband cancellation technique, and/or the harmonic frequency band cancellation techniques described below), such that the spatial components of the total residual magnetic field B_{TOT} at the fine magnetometers 26b drop to a baseline level within the operating range of the fine magnetometers 26b, and subsequently employing a fine feedback control loop 52 having a relatively high sensitivity, but a low dynamic range that encompasses this baseline level for finely cancelling the outside magnetic field B_{OUT} (e.g., low-frequency cancellation of the outside magnetic field B_{OUT} contributed by the Earth's magnetic field, broadband cancellation, and/or the harmonic frequency band cancellation techniques described below), such that the spatial components of the total residual magnetic field B_{TOT} at the fine magnetometers 26b further drop from the baseline level to an even lower level, which can make operation of the magnetometers 26 more reliable. The signal acquisition unit 18 is also configured for managing the coarse feedback control loop 50 and fine feedback control loop 52 by employing a management control loop 54.

In particular, the coarse feedback control loop 50 and fine feedback control loop 52 are implemented in the processor 30, with the coarse feedback control loop 50 coarsely controlling the set of magnetic field actuators 28 in response to input from the coarse magnetometers 26a, and the fine feedback control loop 52 finely controlling the set of magnetic field actuators 28 in response to input from the fine magnetometers 26b. Although the coarse feedback control loop 50 is illustrated as receiving input from three coarse magnetometers 26a, and the fine feedback control loop 52 is illustrated as receiving input from three fine magnetometers 26b, it should be appreciated that the coarse feedback control loop 50 can receive input from more or less coarse magnetometers 26a, including only one coarse magnetometer 26a, and the fine feedback control loop 52 can receive input from more or less fine magnetometers 26b, including only one fine magnetometer 26b. Furthermore, although the coarse feedback control loop 50 and fine feedback control loop 52 are illustrated as receiving input from an equal number of coarse magnetometers 26a and fine magnetometers 26b, the coarse feedback control loop 50 and fine feedback control loop 52 may receive input from an unequal number of coarse magnetometers 26a and fine magnetometers 26b, including a number of coarse magnetometers 26a that is greater or less the number of fine magnetometers 26b.

Initially, due to the relatively low dynamic range of the fine magnetometers 26b, the magnitude of the total residual magnetic field B_{TOT} is too great for the fine magnetometers 26b to detect the total residual magnetic field B_{TOT}. However, due to the relatively high dynamic range of the coarse magnetometers 26a, the spatial components of the total residual magnetic field B_{TOT} can be respectively detected by the coarse magnetometers 26a, which outputs coarse error signals SC_{ERR} corresponding to the spatial components of the detected total residual magnetic field B_{TOT}.

When the magnitude of the total residual magnetic field B_{TOT} is above the dynamic range of the fine magnetometers 26b, the processor 30 acquires the coarse error signals SC_{ERR} output by the coarse magnetometers 26a in response to detecting the spatial components of the total residual magnetic field B_{TOT}, computes the characteristics (namely, the amplitude and phase) of the actuated magnetic field B_{ACT} estimated to minimize the coarse error signals SC_{ERR} output by the coarse magnetometers 26a, and generates a corresponding noise-cancelling control signal C for output to the set of magnetic field actuators 28 for at least partially cancelling the outside magnetic field B_{OUT} at the fine magnetometers 26b, and ultimately suppressing the total residual magnetic field B_{TOT} to a baseline level at the fine magnetometers 26b.

In one embodiment, the processor 30 may estimate the spatial components of the total residual magnetic field B_{TOT} respectively at each fine magnetometer 26b based on the coarse error signals SC_{ERR} output by the coarse magnetometers 26a or fine error signals SF_{ERR} of other fine magnetometers 26b, e.g., using the estimation techniques described in U.S. Provisional Application Ser. No. 62/975,719, entitled “Estimating the Magnetic Field at Distances From Direct Measurements to Enable Fine Sensors to Measure the Magnetic Field from the Brain by Using a Wearable MEG System” (Attorney Docket No. KERN-080PR01), which is expressly incorporated herein by reference.

In the embodiment illustrated in FIG. 3, the set of magnetic field actuators 28 are spatially much closer to the fine magnetometers 26b (and, in fact, may be integrated with the fine magnetometers 26b as a single unit) than the coarse magnetometers 26a. Despite the fact that the coarse magnetometers 26a and fine magnetometers 26b may essentially experience the same outside magnetic field B_{OUT}, due to the spatial differences between coarse magnetometers 26a and fine magnetometers 26b relative to the proximate magnetic field actuators 28, the coarse magnetometers 26a will be affected by the actuated magnetic field B_{ACT} generated by the magnetic field actuators 28 much less than the fine magnetometers 26b will be affected by the same actuated magnetic field B_{ACT} (e.g., 20%).

Hence, in this example, ignoring the minute contribution of the MEG magnetic field B_{MEG} for purposes of simplicity, the coarse magnetometers 26a and fine magnetometers 26b will measure a different total residual magnetic field B_{TOT}=B_{OUT}+B_{ACT}, because even though the outside magnetic field B_{OUT} may be the same at both coarse magnetometers 26a and fine magnetometers 26b, the actuated magnetic field B_{ACT} will differ between the coarse magnetometers 26a and fine magnetometers 26b based on their different proximities to the magnetic field actuators 28. Thus, absent estimation of the spatial components of the total residual magnetic field B_{TOT} respectively at each fine magnetometer 26b, cancellation of the outside magnetic field B_{OUT}, and the resulting suppression of the total residual magnetic field B_{TOT}, at the fine magnetometers 26b based directly (i.e., without correction) on the coarse error signals SC_{ERR} output by the coarse magnetometers 26a may be insufficient.

In accordance with the noise-cancelling control signal C output by the processor 30, the set of magnetic field actuators 28 generates the actuated magnetic field B_{ACT}, which combines with the outside magnetic field B_{OUT} (along with weak MEG magnetic field B_{MEG} from the brain 14) to create a total residual magnetic field B_{TOT} at the fine magnetometers 26b having spatial components that are at baseline level within the operating range of the fine magnetometers 26b.

Once the spatial components of the total residual magnetic field B_{TOT} are at the baseline level, they can be respectively detected by the fine magnetometers 26b, which outputs fine error signals SF_{ERR} corresponding to the spatial components of the detected total residual magnetic field B_{TOT}. The processor 30 then acquires the fine error signals SF_{ERR} output by the fine magnetometers 26b in response to detecting the spatial components of the total residual magnetic field B_{TOT}, computes the characteristics of the actuated magnetic field B_{ACT} estimated to minimize the fine error signals SF_{ERR} output by the fine magnetometers 26b, and generates a corresponding noise-cancelling control signal C for output to the set of magnetic field actuators 28 for at least partially cancelling the outside magnetic field B_{OUT} at the fine magnetometers 26b, and ultimately suppressing the total residual magnetic field B_{TOT} to a lower level than the baseline level at the fine magnetometers 26b.

In one embodiment, even when the spatial components of the total residual magnetic field B_{TOT} are at the baseline level, and the fine error signals SF_{ERR} output by the fine magnetometers 26b are being actively acquired, the processor 30 may be further configured for correcting or refining the fine error signals SF_{ERR} using the estimation techniques described in U.S. Provisional Application Ser. No. 62/975,719, entitled “Estimating the Magnetic Field at Distances From Direct Measurements to Enable Fine Sensors to Measure the Magnetic Field from the Brain by Using a Wearable MEG System” (Attorney Docket No. KERN-080PR01).

In accordance with the noise-cancelling control signal C output by the processor 30, the set of magnetic field actuators 28 generates the actuated magnetic field B_{ACT}, which combines with the outside magnetic field B_{OUT} (along with weak MEG magnetic field B_{MEG} from the brain 14) to create a total residual magnetic field B_{TOT} having spatial components at the fine magnetometers 26b that are at the baseline level. At this point, the fine error signals SF_{ERR} can serve to collect MEG signals S_{MEG} representative of the spatial components of the MEG magnetic field B_{MEG} for further processing by the signal processing unit 20 to identify and localize neural activity in the brain 14 of the user 12.

It should be appreciated that, in the illustrated embodiment, the coarse magnetometers 26a and fine magnetometers 26b are capable of detecting the total residual magnetic field B_{TOT} in three dimensions (x, y, and z), and the set of magnetic field actuators 28 includes three magnetic field actuators 28a-28c (shown in FIG. 2) capable of generating the actuated magnetic field B_{ACT} in three dimensions (x, y, and z). As such, each of the coarse error signals SC_{ERR} and fine error signals SF_{ERR} respectively output by the coarse magnetometers 26a and fine magnetometers 26b to the processor 30, and the control signal C output by the processor 30 to the respective magnetic field actuators 28a-28c, is a vector (i.e., comprises an x-component, y-component, and z-component), such that the outside magnetic field B_{OUT} can be cancelled, and thus the total residual magnetic field B_{TOT} suppressed, in three dimensions.

In an alternative embodiment, the signal acquisition unit 18 (shown in FIG. 4) only employs the coarse feedback control loop 50 for at least partially cancelling the outside magnetic field B_{OUT}, such that the spatial components of the total residual magnetic field B_{TOT} at the fine magnetometers 26b drop to a baseline level within the operating range of the fine magnetometers 26b. In this case, the signal acquisition unit 18a does not have a fine feedback control loop 52, and the processor 30 only uses the coarse error signals SC_{ERR} output by the coarse magnetometers 26a to compute the characteristics of the actuated magnetic field B_{ACT} estimated to suppress the total residual magnetic field B_{TOT} to near-zero at the fine magnetometers 26b, even after the spatial components of the total residual magnetic field B_{TOT} at the fine magnetometers 26b are already at the baseline level, such that the fine magnetometers 26b remain in an operating range.

Whether the signal acquisition unit 18 employs both the coarse feedback control loop 50 and the fine feedback control loop 52 to cancel the outside magnetic field B_{OUT}, or employs only the coarse feedback control loop 50 to cancel the outside magnetic field B_{OUT}, it can be appreciated that the signal acquisition unit 18 is capable of coarsely canceling a large portion of the outside magnetic field B_{OUT}, while still collecting signals from the fine magnetometers 26b sensitive enough to measure the weaker MEG magnetic field B_{MEG} generated by the neural activity in the brain 14 of the user 12.

The processor 30 employs the management control loop 54 to manage how the coarse feedback control loop 50 and fine feedback control loop 52 are employed (e.g., how the coarse error signals SC_{ERR} output by the coarse magnetometers 26a and the fine error signals SF_{ERR} output by the fine magnetometers 26b are to be used) for optimal cancellation of the outside magnetic field B_{OUT}, and thus, optimal suppression of the total residual magnetic field B_{TOT}, and corrects additional factors that can change more slowly over time, such as, e.g., calibrating the magnetometers 26 (e.g., using calibration techniques described in U.S. Provisional Application Ser. No. 62/975,709, entitled “Self-Calibration of Flux Gate Offset and Gain Drift To Improve Measurement Accuracy Of Magnetic Fields From the Brain Using a Wearable MEG System” (Attorney Docket No. KERN-078), which is expressly incorporated herein by reference), and optimizing performance metrics in the signal acquisition unit 18, either globally or locally (e.g., using optimal control methods disclosed in U.S. Provisional Application Ser. No. 62/975,727, entitled “Optimal Methods to Feedback Control and Estimate Magnetic Fields to Enable a Wearable MEG System to Measure Magnetic Fields from the Brain” (Attorney Docket No. KERN-082), which is expressly incorporated herein by reference), adapting to changing time delays in computations, etc. Further details discussing the functioning of the management control loop 54 are disclosed in U.S. Provisional Application Ser. No. 62/975,693, entitled “Nested and Parallel Feedback Control Loops For Ultra-Fine Measurements of Magnetic Fields From the Brain Using a Wearable MEG System” (Attorney Docket No. KERN-079).

The management control loop 54 manages the coarse feedback control loop 50 and fine feedback control loop 52 based on whether the fine magnetometers 26b are in-range or out-of-range, e.g., by considering coarse error signals SC_{ERR} from the coarse magnetometers 26a and ignoring fine error signals SF_{ERR} if the fine magnetometers 26b are out-of-range, and ignoring coarse error signals SC_{ERR} from the coarse magnetometers 26a and considering fine error signals SC_{ERR} from the fine magnetometers 26b if the fine magnetometers 26 are in-range. The management control loop 54 may monitor the spatial component of the total residual magnetic field B_{TOT} and the overall behavior and history of the signal at each fine magnetometer 26b to determine whether or not the fine magnetometer 26b is in-range or out-of-range. It is noted that the spatial components of the total residual magnetic field B_{TOT} at the fine magnetometers 26b may be substantially different from each other, and thus, some of the fine magnetometers 26b may be in-range, while other fine magnetometers 26b may be out-of-range.

With knowledge of whether each of the fine magnetometers 26b are in-range or out-of-range, the management control loop 54 may generally activate the fine feedback control loop 52 after initiating activation of the coarse feedback control loop 50. In this manner, as discussed above, the coarse feedback control loop 50 may coarsely control the actuated magnetic field B_{ACT} in a manner that at least partially cancels the outside magnetic field B_{OUT}, and thus suppresses the total residual magnetic field B_{TOT} at the fine magnetometers 26b to a baseline level, such that the at least one of magnetometers 26b comes in-range. The management control loop 54 may then activate the feedback control loop 52 to finely control the actuated magnetic field B_{ACT} in a manner that further suppresses the total residual magnetic field B_{TOT} at the fine magnetometer(s) 26b that just came in-range to a lower level.

In one embodiment, the management control loop 54 strictly activates only the coarse feedback control loop 50 (e.g., if one of the fine magnetometers 26b is out-of-range) or only the fine feedback control loop (e.g., if all of the fine magnetometers 26 are in-range), but not both the coarse feedback control loop 50 and the fine feedback control loop 52 at the same time. In this case, the management control loop 54 will only consider coarse error signals SC_{ERR} from the coarse magnetometers 26a when the coarse feedback control loop 50 is active, and will only consider fine error signals SF_{ERR} from the fine magnetometers 26b when the fine feedback control loop 52 is active.

In another particularly preferred embodiment, however, the management control loop 54, at any given time, may not strictly activate only the coarse feedback control loop 50 or strictly activate only the fine feedback control loop 52, and thus, both of the coarse feedback control loop 50 and fine feedback control loop 52 may be at least partially activated. The management control loop 54 may choose to consider only the fine error signals SF_{ERR} from the fine magnetometers 26b that are in-range. In this case, the management control loop 54 may determine whether or not the fine magnetometer 26b is in-range, and performs a “sensor hand-off” procedure, and in particular, switches back and forth between consideration of a coarse error signal SC_{ERR} from any given coarse magnetometer 26a and consideration of a fine error signal SF_{ERR} from any given fine magnetometer 26b. It is understood that only some of the fine magnetometers 26b may be out-of-range at any given moment, so the sensor hand-off procedure can be from one, some, or all coarse magnetometers 26a to one, some, or all of the fine magnetometers 26b.

For example, if the management control loop 54 is currently considering a coarse error signal SC_{ERR} from a coarse magnetometer 26, and a previously unavailable fine magnetometer 26b is deemed to be in-range, the processor 30 may then ignore a coarse error signal SC_{ERR} from at least one coarse magnetometer 26a that is in proximity to the previously unavailable fine magnetometer 26b, and instead consider the more accurate fine error signal SF_{ERR} from this previously unavailable fine magnetometer 26b (in essence, passing or handing off detection of the total residual magnetic field B_{TOT} from the coarse magnetometer(s) 26b to the fine magnetometer 26b).

On the contrary, if the management control loop 54 is currently considering a fine error signal SF_{ERR} from a fine magnetometer 26b, and the fine magnetometer 26b is subsequently deemed to fall out-of-range for any one of a variety of reasons (e.g., if the user 12, and thus the fine magnetometer 26b, gets too close to a power outlet, a fridge magnet, a cell phone, or perhaps if the user 12 turns their head so suddenly that the total residual magnetic field B_{TOT} to which the fine magnetometer 26b varies too quickly), the management control loop 54 may then ignore the fine error signal SF_{ERR} from that fine magnetometer 26b, and instead consider the coarse error signal SC_{ERR} from at least one coarse magnetometer 26a in proximity to the now unavailable fine magnetometer 26b (in essence, passing or handing off detection of the total residual magnetic field B_{TOT} from the fine magnetometer 26b to the coarse magnetometer 26a).

Thus, in this manner, the management control loop 54 may operate the fine feedback control loop 52 to control the actuated magnetic field B_{ACT} based on the fine error signals SF_{ERR} respectively output by fine magnetometers 26b as they come in-range. The management control loop 54 may operate the fine feedback control loop 52 to prevent control of the actuated magnetic field B_{ACT} based on the fine error signals SF_{ERR} respectively output by fine magnetometers 26b as they go out-of-range.

In an optional embodiment, the management control loop 54 may weight the fine magnetometers 26b, in which case, the management control loop 54 may not perform a “sensor hand-off” procedure, per se, but may assign a weight a to any given fine magnetometer 26b between a value 0 (no weight) and 1 (full weight). For example, the management control loop 54 may monitor different operating parameters of a fine magnetometer 26b to determine whether the fine magnetometer 26b is in a linear operating range, or outside of the linear operating range, but not saturated (non-linear operating range), or is saturated. If the fine magnetometer 26b is found to be in the linear operating range, the weighting a assigned to the fine magnetometer 26b can be 1 (i.e., full weight); if the fine magnetometer 26b is found to be saturated, the weighting a assigned to the fine magnetometer 26b can be 0 (i.e., no weight); and if the fine magnetometer 26b is found to be in the non-linear operating range, the weighting a assigned to the fine magnetometer 26b can be between 0 and 1 (i.e., partial weight), depending on how close the fine magnetometer 26b is to saturation.

As discussed above, the management control loop 54 is configured for correcting factors that can change more slowly over time to optimize the cancellation of the outside magnetic field B_{OUT}. For example, the management control loop 54 may be configured for implementing adaptions to slow changes of the coarse feedback control loop 50 and fine feedback control loop 52 over time. The management control loop 54 is configured for identifying and determining parameters and coefficients of the signal acquisition unit 18 and the outside magnetic field B_{OUT}. The management control loop 54 is configured for employing computational algorithms to determine unknown parameters from the coarse error signals SC_{ERR} and fine error signals SF_{ERR} output by the coarse magnetometers 26a and fine magnetometers 26b, such as fitting of physical and calibrated mathematical and numerical models to the coarse error signals SC_{ERR} and fine error signals SF_{ERR} to identify missing or insufficiently known coefficients and parameters. Such parameters and coefficients can include offset and gain coefficients for the coarse magnetometers 26a, gain constants for the fine magnetometers 26b, actuator gains and offsets for the set of magnetic field actuators 28, electronics time delay latency coefficients in the coarse feedback control loop 50 and fine feedback control loop 52 (i.e., the amount of time between generating the coarse error signal SC_{ERR} or fine error signal SF_{ERR} and activating the set of magnetic field actuators 28), and other parameters of the signal acquisition unit 18. The management control loop 54 may determine coefficients and parameters for different temporal and spatial ranges. Likewise, the gain that the set of magnetic field actuators 28 may have on the coarse magnetometers 26a and fine magnetometers 26b may differ with the placement and location offset of magnetic field actuators 28 (e.g., as the head of the user 12 moves or the support structure 24 deforms). The management control loop 54 may identify at least one, some, or all of the coefficients or parameters over these changing conditions.

In one exemplary instance, a mathematical and numerical model of the signal acquisition unit 18, or a portion thereof, has some coefficients or parameters that are considered poorly or insufficiently known. In another exemplary instance, a mathematical and numerical model of the signal acquisition unit 18 does not have a predetermined structure, and the coefficients or parameters consist of transfer functions or linear mappings from one set of signals to another. The management control loop 54 may compare the response of a structured or unstructured model of the signal acquisition unit 18 to the measurements from the coarse magnetometers 26a and fine magnetometers 26b, and the coefficients or parameters may be varied until any disagreement between the mathematical model of the signal acquisition unit 18 and the actual measured signals is decreased. The coefficients or parameters of the mathematical model that achieve such a decrease in disagreement are the estimated parameters of the signal acquisition unit 18 (meaning, if the mathematical model with selected parameter values x, y, and z best matches the actual measured behavior of the system, then the values x, y, and z are a system identification estimate of the poorly or insufficiently known coefficients or parameters of the system). In determining the coefficients or parameters of the signal acquisition unit 18, the management control loop 54 may employ weighted least squares, observer filters, Kalman filters, Wiener filters, or other filters. The management control loop 54 may employ time domain, frequency domain, recursive techniques, parametric and non-parametric methods, linear and nonlinear optimization techniques including gradient descent, matrix methods, convex methods, non-convex methods, neural networks, genetic algorithms, fuzzy logic, and machine learning methods.

The management control loop 54 may perform calibration techniques prior to operating the neural activity measurement system 10, or calibration techniques may be performed in real-time as the neural activity measurement system 10 operates. For example, prior to usage, the signal acquisition unit 18 may be calibrated by applying a known magnetic field in a controlled shielded setting (e.g., to characterize the coarse magnetometers 26a for their offsets and gain measurements). However, the properties of coarse magnetometers 26a, fine magnetometers 26b, or set of magnetic field actuators 28 may vary due to environmental variations, such as, e.g., variations in temperature, laser power (for magnetometers that utilize lasers), motion or deformation of the support structure 24, or other deformations, such as bending of the coarse magnetometers 26a, fine magnetometers 26b, or offset of magnetic field actuators 28 due to temperature or mechanical stresses. Thus, in addition to performing calibrations ahead of time, the management control loop 54 may perform calibrations techniques during system operation. For example, if the offsets and gains of the coarse magnetometers 26a change during usage of the neural activity measurement system 10, the management control loop 54 may estimate the offsets and gains of the coarse magnetometers 26a in real time (i.e., as the neural activity measurement system 10 is running), e.g., by estimating and comparing the offset of one coarse magnetometer against the measurements of other coarse or fine magnetometers. Further details discussing the calibration of coarse magnetometers are disclosed in U.S. Provisional Application Ser. No. 62/975,709, entitled “Self-Calibration of Flux Gate Offset and Gain Drift To Improve Measurement Accuracy Of Magnetic Fields From the Brain Using a Wearable MEG System” (Attorney Docket No. KERN-078), which is expressly incorporated herein by reference.

It should be appreciated that, in the case where the signal acquisition unit 18 comprises multiple sets of magnetic field actuators 28 and processors 30, the components, along with the coarse feedback control loop 50, fine feedback control loop 52, and management control loop 54, illustrated in FIG. 4 may be duplicated. In this case, a subset of the coarse magnetometers 26a will be associated with each coarse feedback control loop 50, and a subset of the fine magnetometers 26b will be associated with each fine feedback control loop 52. Because the actuated magnetic field B_{ACT} generated by each set of the magnetic field actuators 28 will affect all of the coarse magnetometers 26a and all of the fine magnetometers 26b, the processors 30 may communicate with each other to generate the proper noise-cancelling control signals C that will result in the composite cancelling magnetic field B_{ACT} to be generated by the combination of sets of magnetic field actuators 28 to cancel the outside magnetic field B_{OUT}. Alternatively, a single processor 30 may be used to control all sets of the magnetic field actuators 26.

Although the total residual magnetic field B_{TOT} may be suppressed to a level that allows the ultra-fine measurements of the MEG magnetic field B_{MEG} emanating from the brain 14 of the user 12 to be taken by the fine magnetometers 26b, some portion of the outside magnetic field B_{OUT} will likely remain in the total residual magnetic field B_{TOT} measured by the fine magnetometers 26b, and thus, will be considered environmental magnetic noise to the relatively weak MEG signals S_{MEG} contained in the measured total residual magnetic field B_{TOT}.

Significantly, although the measurement errors of fine magnetometers 26b are relatively small, the processor 30 is configured for distinguishing the portion of the measured total residual magnetic field B_{TOT-MEAS} that corresponds to the true MEG magnetic field B_{MEG-TRUE} (i.e., the true magnetic field that emanates from the head of the user 12 due to neural activity in the brain 14) and the portion of the measured total residual magnetic field B_{TOT-MEAS} that does not correspond to the true MEG magnetic field B_{MEG-TRUE} by employing a combination of three signal discriminating techniques, thereby maximizing the accuracy of the measurements of these fine magnetometers 26b.

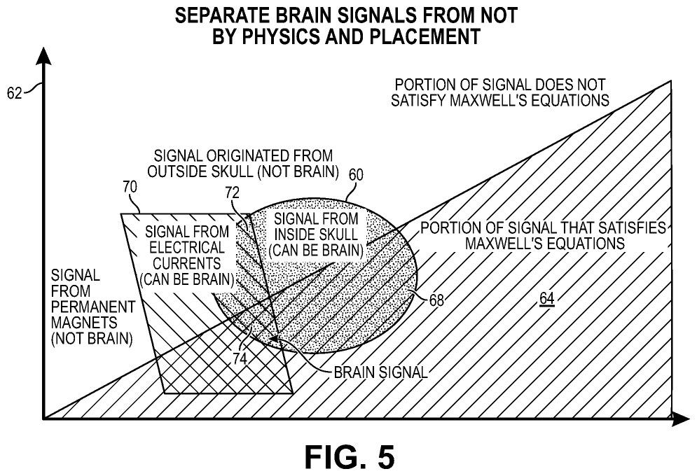

In particular, referring to FIG. 5, the processor 30 is configured for distinguishing the portion of the measured total residual magnetic field B_{TOT-MEAS} that corresponds to the MEG magnetic field B_{MEG} (representing by the space in the oval 60) and the portion of the measured total residual magnetic field B_{TOT-MEAS} corresponding to the outside magnetic field B_{OUT} (represented by the space in the rectangle 62, but outside the oval 60) based on the strength, temporal frequency, and spatial frequency of typical MEG magnetic fields B_{MEG} and typical outside magnetic field B_{OUT}.

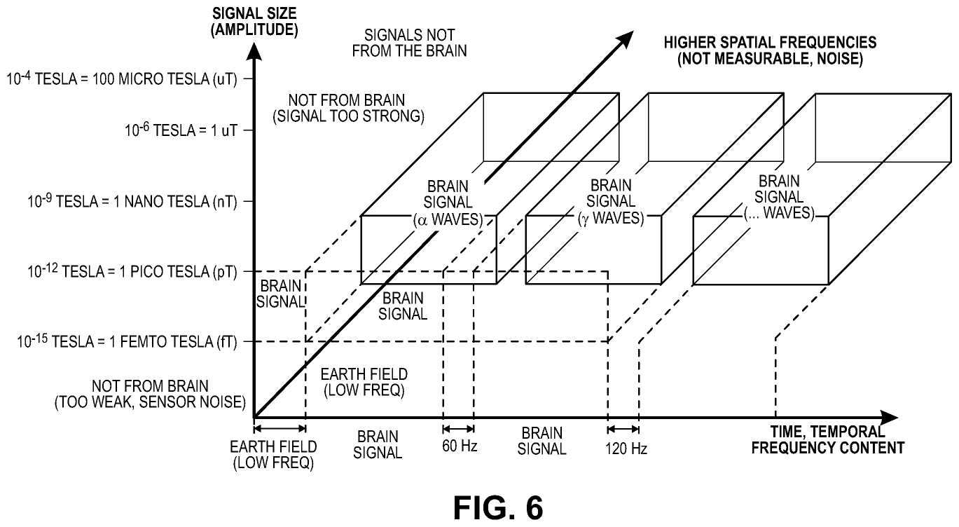

For example, as illustrated in FIG. 6, typical strength components, temporal frequency components, and spatial frequency components of an exemplary MEG magnetic field B_{MEG} (which contains α waves, γ waves, and other waves), an exemplary outside magnetic field B_{OUT}, and an exemplary measurement noise δ, which are based on known and typical properties of MEG magnetic fields, outside magnetic fields, and measurement noise δ, are shown in a three-dimensional plot having a size (vertical) axis, temporal frequency (horizontal) axis, and a spatial frequency (diagonal) axis.

With regard to the size (vertical) axis of FIG. 6, it can be seen that the exemplary MEG magnetic field B_{MEG} (in the femto tesla (f) range) comprises a range of strength components that is substantially lower than the range of strength components in the exemplary outside magnetic field B_{OUT} (at least in the pico tesla (pT) range) and substantially higher than the range of strength components in the exemplary measurement noise δ (below the femto tesla (f) range). Thus, based on this, it is known that a strength component of a measured total residual magnetic field B_{TOT-MEAS} that is substantially greater than the femto tesla (f) range is too strong to correspond to the MEG magnetic field B_{MEG}, and instead corresponds to the outside magnetic field B_{OUT}, while a strength component in the measured total residual magnetic field B_{OUT-MEAS} that is substantially less than the femto tesla (f) range is too weak to correspond to the MEG magnetic field B_{MEG}, and instead correspond to measurement noise δ.

Thus, it can be appreciated from FIG. 6 that the MEG magnetic field B_{MEG}, the outside magnetic field B_{OUT}, and the measurement noise δ may be distinguished from each other based on size. size. Based on this knowledge, the processor 30 may reduce the content of the outside magnetic field B_{OUT} and measurement noise δ in the measured total residual magnetic field B_{TOT-MEAS} by eliminating the content of the measured total residual magnetic field B_{TOT-MEAS} corresponding to strength components above and below the pico tesla (pT) range.

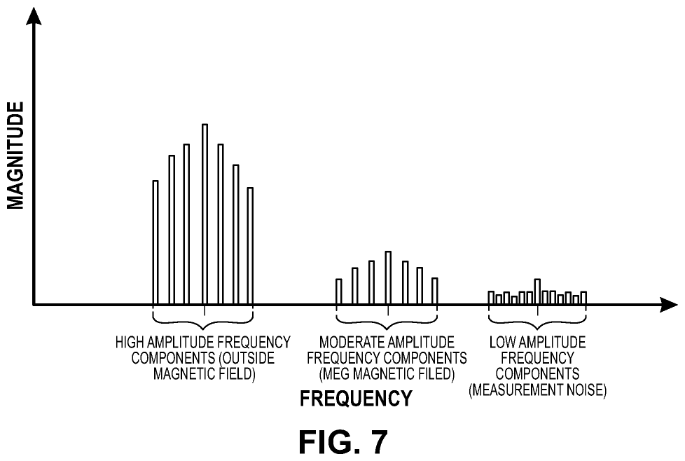

In one embodiment, the processor 30 accomplishes this by transforming the measured total residual magnetic field B_{OUT-MEAS} from a time domain into the frequency domain (e.g., using a Fast Fourier Transform (FFT), and eliminating the content of the measured total residual magnetic field B_{TOT-MEAS} corresponding to the frequency components having peak amplitudes that are above and below the pico tesla (pT) range. For example, as illustrated in FIG. 7, the frequency domain of an exemplary measured total residual magnetic field B_{TOT-MEAS} comprises frequency components having a relatively high peak amplitude (likely corresponding to the outside magnetic field B_{OUT}), frequency components having a relatively low peak amplitude (likely corresponding to measurement noise δ), and frequency components having a relatively moderate peak amplitude (likely corresponding to the MEG magnetic field B_{MEG}) The processor 30 may then eliminate the content of the total residual magnetic field B_{TOT-MEAS} corresponding to the frequency components having a relatively high peak amplitude and a relatively low peak amplitude.

The processor 30 may accomplish this by filtering the measured total residual magnetic field B_{TOT-MEAS} at these frequency components, and cancelling the outside magnetic field B_{OUT} at these frequency components (e.g., by generating cancellation control signals based on this determination that are output to the set of magnetic field actuators 28 and actuating the set of magnetic field actuators 28 in accordance with these cancellation control signals. Alternatively, the processor 30 may eliminate the content of the measured total residual magnetic field B_{TOT-MEAS} corresponding to certain frequency components by filtering these frequency components out of the measured total residual magnetic field B_{TOT-MEAS} during a post-cancellation step.

It should be appreciated that the strength thresholds of the outside magnetic field B_{OUT}, MEG magnetic field B_{MEG}, and measurement noise δ may vary. For example, the distance from the brain 14 and the location of the fine magnetometers 26b may change, because different individuals may have different skull, skin (i.e., scalp), and hair thicknesses, and because the fine magnetometers 26b may be in direct contact with the scalp or may be set back from the scalp (e.g., to accommodate additional elements in the wearable signal processing unit 18, or for thermal management reasons). As the distance of a fine magnetometer 26b and the brain 14 increases, the strength of the MEG magnetic field B_{MEG} at that fine magnetometer 26b decreases, and thus, the strength threshold at which moderate strength components of the measured total residual magnetic field B_{OUT-MEAS} is selected relative to the too-strong or too-weak strength components of the measured total residual magnetic field B_{OUT-MEAS} may be modified to account for the collective distance between the fine magnetometers 26b and the brain 14 of the user 12.

With regard to the temporal frequency (horizontal) axis of FIG. 6, it can be seen that the exemplary MEG magnetic field B_{MEG} comprises a range of temporal frequency components (in the range of a few Hertz to a few hundred Hertz) that is substantially higher than the range of temporal frequency components (DC (constant in time) to a few Hertz) in the exemplary outside magnetic field B_{OUT} (in the range of a few Hertz to a few hundred Hertz) and substantially lower than the range of temporal frequency components (in the range of thousands of Hertz) in the exemplary measurement noise δ. The DC and low temporal frequency components of the outside magnetic field B_{OUT} are due to the Earth's magnetic field, which does not change appreciably in time. It should be appreciated that, although portions of the exemplary MEG magnetic field B_{MEG} (namely, the α waves, γ waves, and other waves) has been illustrated as being discretely spaced apart for purposes of illustration, temporal frequency spectrum of the MEG magnetic field B_{MEG} is a continuum.

The outside magnetic field B_{OUT} also comprises temporal frequency components at 60 Hz due to time-varying electromagnetic radiation emanating from electrical outlets and sockets, electrical wires or connections in the wall, and everyday electrical equipment when the user 12 is in a home, work, or laboratory setting in the United States, as well as at multiples of 60 Hz due to non-linear interactions between the electromagnetic radiation and environment (as the electromagnetic field couples with everyday objects, e.g., metal spars in a chair or table or refrigerator) that lead to frequency doublings, triplings, etc.

It should be appreciated that the harmonic components in the outside magnetic field B_{OUT} may be in multiples of a frequency that differs from 60 Hz. For example, the harmonic components may be in multiples of 50 Hz if the home, work, or laboratory setting is in Europe. Also, due to variations and imperfections in the power supply to electronics, the harmonic components in the outside magnetic field B_{OUT} may be in multiples of a frequency that is exactly 60 Hz (or 50 Hz). Furthermore, due to coupling of the electromagnetic radiation with the environment, some frequency spread may occur, such that the finite frequency bands centered at (or approximately at) 60 Hz, 120 Hz, 180 Hz, etc. (or 50 Hz, 100 Hz, 150 Hz, etc.) occur in the outside magnetic field B_{OUT}.

Based on this knowledge, the processor 30 may reduce the content of the outside magnetic field B_{OUT} and measurement noise δ in the measured total residual magnetic field B_{TOT-MEAS} by eliminating the content of the measured total residual magnetic field B_{TOT-MEAS} corresponding to temporal frequency components in the range of DC to a few Hertz, in the range of thousands of Hertz, and also at harmonic temporal frequencies of 60 Hz (or 50 Hz).

The processor 30 may accomplish this by filtering the measured total residual magnetic field B_{TOT-MEAS} at these temporal frequency components, and cancelling the outside magnetic field B_{OUT} at these temporal frequency components (e.g., by generating cancellation control signals based on this determination that are output to the set of magnetic field actuators 28 and actuating the set of magnetic field actuators 28 in accordance with these cancellation control signals. Further details discussing cancelling the outside magnetic field B_{OUT} at selected temporal frequency components are disclosed in U.S. Provisional Application Ser. No. 62/975,693, entitled “Nested and Parallel Feedback Control Loops For Ultra-Fine Measurements of Magnetic Fields From the Brain Using a Wearable MEG System” (Attorney Docket No. KERN-079), which is expressly incorporated herein by reference. Alternatively, the processor 30 may eliminate the content of the measured total residual magnetic field B_{TOT-MEAS} corresponding to certain temporal frequency components by filtering these temporal frequency components out of the measured total residual magnetic field B_{TOT-MEAS} during a post-cancellation step.

With regard to the spatial frequency (diagonal) axis of FIG. 6, it can be seen that the exemplary MEG magnetic field B_{MEG} comprises a range of spatial frequency components that is substantially higher than the range of spatial frequency components in the exemplary outside magnetic field B_{OUT} and substantially lower than the range of spatial frequency components in the exemplary measurement noise δ.

Just like a magnetic field can have temporal frequency components (e.g., slow or less than 5 Hz), three-dimensional magnetic fields also have spatial frequency components (long spatial wavelength (e.g. greater than 1 meter) corresponds to low spatial frequency (e.g., less than 1 cycle/meter)). Thus, a magnetic field may oscillate over short distances in space (short wavelength) or oscillate over long distances (long wavelengths).

The Earth's magnetic field has a low spatial frequency. For example, magnetic North is basically the same on one side of a room as it is on the other side of the room. When the Earth's magnetic field interacts with everyday objects that have magnetizable components; for example, a chair leg, table spar or a beam in the wall of a room that is composed of metal, such as ferrous iron, that is magnetically responsive, such everyday objects may modify the Earth's magnetic field (a bending and curvature of Earth's magnetic field) or equivalently the magnetic flux lines. Thus, in a home, office, or laboratory environment, the Earth's magnetic field may spatially vary in the vicinity of the magnetizable metals or other magnetizable materials. However, the Earth's magnetic field is only modified modestly by magnetizable components, and unless the signal acquisition unit 18 is very close to a magnetizable component (e.g., if the user 12 places their head right next to an iron leg), the modestly modified Earth's magnetic field will be almost constant across the head of the user 12 or, if more accuracy is desired, may be represented accurately by a constant (0^{th} order) component plus a linear (1^{st} order) component. If a compass was held at one side or the other side of the head of the user 12, the direction and strength of the Earth's magnetic field, would be about the same for both sides of the user of the user 12. Hence, the resulting spatial frequencies in an outside magnetic field B_{OUT} corresponding to the Earth's magnetic field, even in an indoor or outdoor setting where there are magnetizable materials in the vicinity, are still typically small.

Likewise, even though the time-varying electromagnetic radiation emanating from electrical outlets and sockets, electrical wires or connections in the wall, and everyday electrical equipment when the user 12 is in a home, work, or laboratory setting has fast varying harmonic temporal frequency components, the spatial short wavelength components of this electromagnetic radiation quickly dissipates with distance from the source or sources of the electromagnetic radiation. Thus, as long as the signal acquisition unit 18 is not adjacent to the source of the electromagnetic energy, then the spatial short wavelength components of the electromagnetic radiation will have dissipated by the time the electromagnetic radiation has reach the head of the user 12. Thus, the spatial frequency components of the outside magnetic field B_{OUT} will generally be relatively low.

In contrast, the spatial frequency components of the MEG magnetic field B_{MEG} will generally be higher at the head of the user 12 than those of the outside magnetic field B_{OUT}. Neurons in the brain 16 of the user 12 that produce the electrical currents are packed closely together, such that they create a MEG magnetic field B_{MEG} with short wavelength components.

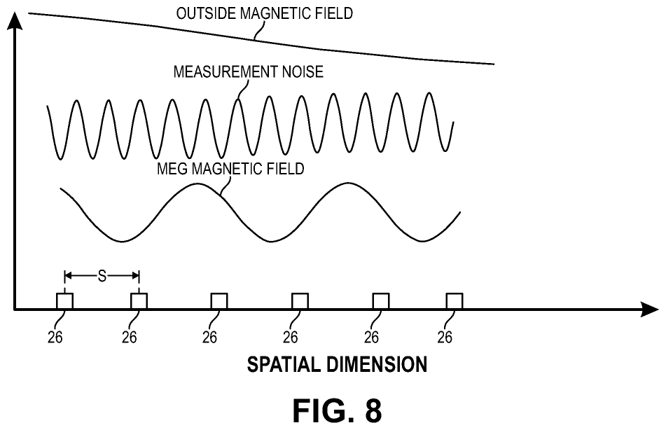

Based on this knowledge, the processor 30 may reduce the content of the outside magnetic field B_{OUT} in the measured total residual magnetic field B_{TOT-MEAS} by eliminating the content of the measured total residual magnetic field B_{TOT-MEAS} corresponding to low spatial frequency components. For example, referring to FIG. 8, a plurality of magnetometers 26 extending along a single axis is illustrated, although it should be appreciated that the magnetometers 26 are arranged relative to each other in three-dimensional space. The spatial frequency of the outside magnetic field B_{OUT} across these magnetometers 26 is relatively low, while the spatial frequency of an exemplary MEG magnetic field B_{MEG} across these magnetometers 26 is relatively high.

The processor 30 collectively processes the spatial components of the total residual magnetic field B_{TOT-MEAS} measured by the magnetometers 26 in a manner that cancels the content of the outside magnetic field B_{OUT} from the measured total residual magnetic field B_{TOT-MEAS}. For example, the spatial components of the magnetometers 26 may be averaged to acquire a DC level that can then be individually subtracted from the spatial components of the measured total residual magnetic field B_{TOT-MEAS}, thereby reducing the content of the outside magnetic field B_{OUT} in the measured total residual magnetic field B_{TOT-MEAS}.

Referring back to FIG. 8, the spatial frequency of exemplary sensor noise δ across these magnetometers 26 is higher than the spatial frequency of the exemplary MEG magnetic field B_{MEG}. The magnetometers 26 are spaced at some maximum spatial frequency (i.e., the maximum spatial sampling frequency) set by their size, e.g., spaced 1 cm, 1 mm, 0.1 mm, or some other suitable spacing s. Magnetic fields having spatial frequency components higher than or equal to the spatial sampling frequency of the magnetometers 26 (i.e., shorter wavelength than the spacing s of the magnetometers 26), are not well-sampled and will be well represented in the measured total residual magnetic field B_{TOT-MEAS}. As the spacing s between the magnetometers 26 is decreased, higher spatial frequency components of the MEG magnetic field B_{MEG} will be contained in the measured total residual magnetic field B_{TOT-MEAS}.

It should be appreciated that although the processor 30 may be configured for distinguishing the MEG magnetic field B_{MEG}, outside magnetic field B_{OUT}, and measurement noise δ based on any combination of strength, temporal frequency, and spatial frequency, and in any order, the processor 30 may be configured for selecting the combination and order of strength, temporal frequency, and spatial frequency on which to distinguish the MEG magnetic field B_{MEG}, outside magnetic field B_{OUT}, and measurement noise δ based on certain criteria.

There may be conditions where the strength components, temporal frequency components, or spatial frequency components of the MEG magnetic field B_{MEG} coincide with the strength components, temporal frequency components, or spatial frequency components of the outside magnetic field B_{OUT} or measurement noise δ, in which case, the processor 30 may not opt to not eliminate content of the outside magnetic field B_{OUT} and/or measurement noise δ based on the coinciding strength, temporal frequency, and/or spatial frequency.

As one example, in the case where MEG magnetic field B_{MEG} and the outside magnetic field B_{OUT} are distinguished based on the strength or temporal frequency, eliminating content of the measured total residual magnetic field B_{TOT-MEAS} may inadvertently eliminate underlying content of the MEG magnetic field B_{MEG} corresponding to frequency components that coincide with the frequency components at which the content of the measured total residual magnetic field B_{TOT-MEAS} has been eliminated, e.g., at 60 Hz or 120 Hz.

In this case, the content of the measured total residual magnetic field B_{TOT-MEAS} corresponding to these frequency components may instead be retained in the measured total residual magnetic field B_{TOT-MEAS}, and may be eliminated from the measured total residual magnetic field B_{TOT-MEAS} in a different regime. For example, it is likely that the content of the outside magnetic field B_{OUT} corresponding to the same frequency components of the underlying content of the MEG magnetic field B_{MEG} has been contributed by electromagnetic radiation from electrical equipment or power sources that has a low spatial frequency in contrast to the high spatial frequency of the MEG magnetic field B_{MEG}.

Thus, the processor 30 may opt to distinguish the MEG magnetic field B_{MEG} and the outside magnetic field B_{OUT} based on spatial frequency, in which case, it can eliminate at least portion of the content of the outside magnetic field B_{OUT} from the measured total residual magnetic field B_{TOT-MEAS} without eliminating the content of the MEG magnetic field B_{MEG} as discussed above.

As another example, due to the interaction between the electromagnetic radiation and the environment, the strength of the harmonic frequency components in the outside magnetic field B_{OUT} may have not always be at a relatively high amplitude, but can be at a relatively low amplitude commensurate with the strength of the MEG magnetic field B_{MEG}. In such case, the MEG magnetic field B_{MEG} and the outside magnetic field B_{OUT} may not be distinguished from each other based on strength, as discussed above. The MEG magnetic field B_{MEG} and the outside magnetic field B_{OUT} may also not be distinguished based on temporal frequency, since the harmonic frequency components of the outside magnetic field B_{OUT} are likely to coincide with frequency components of the MEG magnetic field B_{MEG}.

However, the MEG magnetic field B_{MEG} and the outside magnetic field B_{OUT} may be distinguished from each other based on spatial frequency even when the strength and harmonic frequency components of the outside magnetic field B_{OUT} are commensurate with the strength and frequency components of the MEG magnetic field B_{MEG}. Thus, the processor 30 may opt to distinguish the MEG magnetic field B_{MEG} and the outside magnetic field B_{OUT} based on spatial frequency, in which case, it can eliminate at least portion of the content of the outside magnetic field B_{OUT} from the measured total residual magnetic field B_{TOT-MEAS} without eliminating the content of the MEG magnetic field B_{MEG} as discussed above.

The processor 30 may be configured for dynamically selecting the combination and order of strength, temporal frequency, and spatial frequency on which to distinguish the MEG magnetic field B_{MEG}, outside magnetic field B_{OUT}, and measurement noise δ based on criteria other than the inadvertent coincidence of strength, temporal frequency, or spatial frequency between the MEG magnetic field B_{MEG} and the outside magnetic field B_{OUT} or measurement noise δ.

For example, distinguishing between the MEG magnetic field B_{MEG} and the outside magnetic field B_{OUT} and measurement noise δ based on temporal frequency relies on priori knowledge that the MEG magnetic field B_{MEG} and the outside magnetic field B_{OUT} and measurement noise δ have certain dominant temporal frequency components. In contrast, while the preferred embodiment of distinguishing between the MEG magnetic field B_{MEG} and the outside magnetic field B_{OUT} and measurement noise δ based on strength relies on analyzing the frequency components of the measured total residual magnetic field B_{TOT-MEAS} in the frequency domain, such technique does not rely on prior knowledge that the highest strength of the MEG magnetic field B_{MEG} and the outside magnetic field B_{OUT} and measurement noise δ is at certain temporal frequency components.

Although there may be some expectation that certain frequency components of the measured total residual magnetic field B_{TOT-MEAS} analyzed in the frequency domain will be dominant, and thus may coincide with the same temporal frequency components that the measured total residual magnetic field B_{TOT-MEAS} will be used to distinguish between the MEG magnetic field B_{MEG} and the outside magnetic field B_{OUT} and measurement noise δ based on temporal frequency, thereby may be unexpected dominant frequency components in the frequency domain of the measured total residual magnetic field B_{TOT-MEAS} that do not coincide with the temporal frequency components that will be, or have been, used to distinguish between the MEG magnetic field B_{MEG} and the outside magnetic field B_{OUT} and measurement noise δ based on temporal frequency (e.g., if at least a portion of the content of the outside magnetic field B_{OUT} corresponds electromagnetic radiation having temporal frequency components that are not at DC or the 60 Hz harmonic components).

Thus, the processor 30 may opt to first distinguish MEG magnetic field B_{MEG} and the outside magnetic field B_{OUT} and measurement noise δ based on temporal frequency, such that at least some of the content of the outside magnetic field B_{OUT} and measurement noise δ is eliminated from the measured total residual magnetic field B_{TOT-MEAS}. If the measured total residual magnetic field B_{TOT-MEAS}, after such content has been eliminated, is still too high, the processor 30 may opt to then distinguish MEG magnetic field B_{MEG} and the outside magnetic field B_{OUT} and measurement noise δ based on strength, such that more content of the outside magnetic field B_{OUT} and measurement noise δ is eliminated from the measured total residual magnetic field B_{TOT-MEAS}. If the measured total residual magnetic field B_{TOT-MEAS}, after such additional content has been eliminated, is still too high, the processor 30 may opt to then distinguish MEG magnetic field B_{MEG} and the outside magnetic field B_{OUT} and measurement noise δ based on spatial frequency, such that even more content of the outside magnetic field B_{OUT} and measurement noise δ is eliminated from the measured total residual magnetic field B_{TOT-MEAS}.

The processor 30 may opt to dynamically select whether or not to eliminate content of the outside magnetic field B_{OUT} or measurement noise δ from the measured total residual magnetic field B_{TOT-MEAS} based on practical considerations, even after properly distinguishing the MEG magnetic field B_{MEG}, outside magnetic field B_{OUT}, and measurement noise δ. For example, due to complex factors, the outside magnetic field B_{OUT} may comprise strong frequency components at 60 Hz, 120 Hz, and 240 Hz, but a weak frequency component at 180 Hz. In this instance, the processor 30 may opt to eliminate the content of the outside magnetic field B_{OUT} from the measured total residual magnetic field B_{TOT-MEAS} at 60 Hz, 120 Hz, and 240 Hz, but not at 240 Hz if it is deemed that attempting to eliminate the content of the outside magnetic field B_{OUT} from the measured total residual magnetic field B_{TOT-MEAS} at 240 Hz would add more noise to the measured total residual magnetic field B_{TOT-MEAS} than the noise created by the 240 Hz frequency component of the outside magnetic field B_{OUT}.

Referring back to FIG. 5, the processor 30 is further configured for using Maxwell's equations to distinguish between the portion of the measured total residual magnetic field B_{TOT-MEAS} corresponding to the true total residual magnetic field B_{TOT} (i.e., the physical portion that satisfies Maxwell's equations, which is represented by the space in the bottom triangle 64) and the portion of the measured total residual magnetic field B_{TOT-MEAS} corresponding to measurement errors (i.e., the non-physical portion that does not satisfy Maxwell's equations, which is represented by the space in the top triangle 66). In this manner, the non-physical portion of the measured total residual magnetic field B_{TOT-MEAS} may be eliminated, or at least substantially reduced. This should be contrasted with previous techniques that utilize Maxwell's equations to separate portions of a magnetic field in space, e.g., signals that originate from in the brain and signals that originate from outside of the brain, but are not capable of distinguishing the physical portion of the signals that originate from the brain from the non-physical portion that originates from the brain.

Thus, the processor 30 is configured for correcting the measurement errors in the environmental magnetic field B_{ENV} component of the total residual magnetic field measurements B_{TOT-MEAS}, thereby increasing the accuracies of the estimates of the total residual magnetic field B_{TOT} at the fine magnetometers 26b. As a result of reducing measurements errors associated with the outside magnetic field B_{OUT} component in the total residual magnetic field measurements B_{TOT-MEAS}, the outside magnetic field B_{OUT} may be more accurately cancelled, thereby more effectively suppressing the total residual magnetic field B_{TOT} at the fine magnetometers 26b to bring the fine magnetometers 26b in-range. Furthermore, as a result reducing measurements errors associated with the MEG magnetic field B_{MEG} component in the total residual magnetic field measurements B_{TOT-MEAS}, the MEG magnetic field B_{MEG} may be more accurately determined. In effect, the physical (true) portion of the MEG magnetic field B_{MEG} component of the measured total residual magnetic field B_{TOT-MEAS} and the non-physical (error) portion of the MEG magnetic field B_{MEG} component of the measured total residual magnetic field B_{TOT-MEAS} is distinguished, as represented by the union space 68 between the oval 60 and the bottom triangle 64.

To this end, the processor 30 is configured for inferring total residual magnetic field estimates B_{TOT-EST} at the magnetometers 26 by (1) acquiring the measurements of the total residual magnetic field B_{TOT-MEAS} from the magnetometers 26 (i.e., the coarse error signals SC_{ERR} and/or fine error signals SF_{ERR}); (2) determining the known actuated magnetic field B_{ACT-KNOWN} at the magnetometers 26 based on a known profile of the set of magnetic field actuators 28 and the actuation strengths of the magnetic field actuators 28; (3) generating a generic model of the environmental magnetic field B_{ENV-MOD} in the vicinity of the magnetometers 26; (4) constraining the environmental magnetic field model B_{ENV-MOD} to generate a Maxwell-constrained model of the environmental magnetic field B_{ENV-MAXWELL} that satisfies Maxwell's equations; (5) parameterizing the Maxwell-constrained environmental magnetic field model B_{ENV-MAXWELL} based on the measured total residual magnetic field B_{TOT-MEAS} measured by the magnetometers 26 and the known actuated magnetic field B_{ACT-KNOWN} at the magnetometers 26 to generate a parameterized environmental magnetic field model B_{ENV-PAR} (representative of the true environmental magnetic field model B_{ENV} in the vicinity of the magnetometers 26); (6) determining the environmental magnetic field estimates B_{ENV-EST} at the magnetometers 26 based on the parameterized environmental magnetic field model B_{ENV-PAR}; and (7) determining the total residual magnetic field estimates B_{TOT-EST} at the magnetometers 26 based on the known actuated magnetic field B_{ACT-KNOWN} at the magnetometers 26 and the environmental magnetic field estimates B_{ENV-EST} at the magnetometers 26.

As described below, the total residual magnetic field estimates B_{TOT-EST} are inferred at both the coarse magnetometers 26a and fine magnetometers 26b based on total residual magnetic field measurements B_{TOT-MEAS} acquired from both the coarse magnetometers 26a and fine magnetometers 26b, but in alternative embodiments, the total residual magnetic field estimates B_{TOT-EST} may be inferred at only the coarse magnetometers 26a based on total residual magnetic field measurements B_{TOT-MEAS} acquired from both the coarse magnetometers 26a and fine magnetometers 26b, or may be inferred at only the fine magnetometers 26b based on total residual magnetic field measurements B_{TOT-MEAS} acquired from only the fine magnetometers 26b.

With regard to acquiring the total residual magnetic field measurements B_{TOT-MEAS} from the magnetometers 26, in an exemplary embodiment, an N^{c} number of coarse magnetometers 26a respectively at an N^{c} number of locations may collect an N^{c}×K coarse measurements of the total residual magnetic field B_{TOT-MEAS}^{C} over time in accordance with the discretized matrix:

$${B}_{TOT - MEAS}^{C} = [ \begin{matrix}{B}_{TOT - {MEAS}_{11}}^{C} & … & {B}_{TOT - {MEAS}_{1 K}}^{C} \\ ⋮ & ⋱ & ⋮ \\ {B}_{TOT - {MEAS}_{N} {c}_{1}}^{C} & … & {B}_{TOT - {MEAS}_{N} {c}_{K}}^{C}\end{matrix} ] . \qquad [ 1 a ]$$

Similarly, an N^{F} number of fine magnetometers 26b respectively at an N^{F} number of locations collect an N^{F}×K fine measurements of the total residual magnetic field K_{TOT-MEAS}^{F} over time in accordance with the discretized matrix:

$${B}_{TOT - MEAS}^{F} = [ \begin{matrix}{B}_{TOT - {MEAS}_{11}}^{F} & … & {B}_{TOT - {MEAS}_{1 K}}^{F} \\ ⋮ & ⋱ & ⋮ \\ {B}_{TOT - {MEAS}_{N} {F}_{1}}^{F} & … & {B}_{TOT - {MEAS}_{N} {F}_{K}}^{F}\end{matrix} ] . \qquad [ 1 b ]$$

One of ordinary skill in the art of control and signal processing will recognize that the timing of the coarse N^{c}×K total residual magnetic field measurements B_{TOT-MEAS}^{C} taken by the coarse magnetometers 26a and the fine N^{F}×K total residual magnetic field measurements B_{TOT-MEAS}^{F} taken by the fine magnetometers 26b need not be the same, and that the coarse N^{c}×K total residual magnetic field measurements B_{TOT-MEAS}^{C}, N^{F}×K total residual magnetic field measurements B_{TOT-MEAS}^{F}, and M×K actuations of the actuated magnetic field B_{ACT} may be performed at the same time and may be non-synchronized.

Each of the coarse N^{c}×K total residual magnetic field measurements B_{TOT-MEAS}^{C}, N^{F}×K total residual magnetic field measurements B_{TOT-MEAS}^{F}, and M×K actuations of the actuated magnetic field B_{ACT} is known imperfectly. For example, although each of the fine N^{F}×K total residual magnetic field measurements B_{TOT-MEAS}^{F}, may have a relatively high accuracy, each of the fine magnetometers 26b still have a measurement variance on the order of picoteslas (pT). In contrast, each of the coarse N^{c}×K total residual magnetic field measurements B_{TOT-MEAS}^{C} has a relatively low accuracy, and in particular, each of the coarse magnetometers 26a may have a much higher measurement variance on the order of microteslas (μT) or tens or hundreds of microteslas (μT).

For the purposes of the following discussion, the coarse N^{c}×K total residual magnetic field measurements B_{TOT-MEAS}^{C} and fine N^{F}×K total residual magnetic field measurements B_{TOT-MEAS}^{F} can be consolidated into an N× K number of total residual magnetic field measurements B_{TOT-MEAS} (the sum of the N^{c} number of coarse magnetometers 26a and the N^{F} number of fine magnetometers 26b (if available)), such that equations [1a] and [1b] reduces to:

$${B}_{TOT - MEAS} = [ \begin{matrix}{B}_{TOT - {MEAS}_{11}} & … & {B}_{TOT - {MEAS}_{1 K}} \\ ⋮ & ⋱ & ⋮ \\ {B}_{TOT - {MEAS}_{N 1}} & … & {B}_{TOT - {MEAS}_{NK}}\end{matrix} ] . \qquad [ 1 ]$$

Assuming that the magnetometers 26 are vector magnetometers for respectively measuring the x-, y-, and z-components of the total residual magnetic field measurements B_{TOT-MEAS}, equation [1] can be expressed as a vector \vec{B_{TOT-MEAS}}(x, y, z, t) that varies over space and time, where x, y, z are the three cardinal directions, and t is time that varies over space and time.

The total residual magnetic field measurements \vec{B_{TOT-MEAS}}(x, y, z, t) at the locations of an N number of magnetometers 26 may be given as:

$$[ \begin{matrix}\vec{{B}_{TOT \text{-} MEAS}} ( x , y , z , t ) , 1 \\ \vec{{B}_{TOT \text{-} MEAS}} ( x , y , z , t ) , 2 \\ \\ \\ \\ \vec{{B}_{TOT \text{-} MEAS}} ( x , y , z , t ) , N\end{matrix} ] \qquad [ 2 ]$$

As briefly discussed above, the processor 30 may determine the known actuated magnetic field B_{ACT-KNOWN} at the magnetometers 26 based on a known profile of the set of magnetic field actuators 28 and the actuation strengths of the magnetic field actuators 28. In an exemplary embodiment, an M number of the magnetic field actuators 28 may apply an M×K actuations of the actuated magnetic field B_{ACT} over time in accordance with the discretized matrix:

$${B}_{A C T} = [ \begin{matrix}{B}_{{ACT}_{11}} & … & {B}_{{ACT}_{1 K}} \\ ⋮ & ⋱ & ⋮ \\ {B}_{{ACT}_{M 1}} & … & {B}_{{ACT}_{MK}}\end{matrix} ] . \qquad [ 3 ]$$

Assuming that the set of magnetic field actuators 28 comprises a triad of uniform magnetic field actuators 28a-28c (M=3) for respectively generating x-, y-, and z-components of the actuated magnetic field B_{ACT} to cancel the outside magnetic field B_{OUT} in all three dimensions, the actuated magnetic field B_{ACT} can be defined as a vector \vec{B_{ACT}}(x, y, z, t) that varies over space and time.

The set of magnetic field actuators 28 respectively have an M number of actuation strengths in the form of a vector [symbol] (t) (one for each magnetic field actuator 28) and a matrix of influence R by the actuation strength vector [symbol] (t) to the actuated magnetic field \vec{B_{ACT}}(x, y, z, t) at the N number of magnetometers 26, as follows:

$$R = [ \begin{matrix}{R}_{11} & … & {R}_{1 M} \\ ⋮ & ⋱ & ⋮ \\ {R}_{N 1} & … & {R}_{NM}\end{matrix} ] . \qquad [ 4 ]$$

The matrix of influence R may be generated using mathematical or numerical modeling (e.g., by simulating the magnetic field emanating from each of the magnetic field actuators 28 to different spatial locations, e.g., at the magnetometers 26) or by the performance of calibration measurements ahead of time (i.e., generate a nominal actuated magnetic field and measure the actuated magnetic field at different spatial locations, e.g., at the magnetometers 26) that quantifies the profile of the actuated magnetic field B_{ACT} generated by each of magnetic field actuators 28, and therefore defines the influence of each magnetic field actuator 28 at the location of each magnetometer 26. The resulting actuated magnetic field at the locations of the magnetometers 26 will linearly scale with the actuation strength vectors j(t) of the magnetic field actuators 28, such that a known actuated magnetic field \vec{B_{ACT-KNOWN}}(x, y, z, t) that varies over space and time at the N number of magnetometers 26 may be given as:

$$[ \begin{matrix}\vec{{B}_{ACT \text{-} KNOWN}} ( x , y , z , t ) , 1 \\ \vec{{B}_{ACT \text{-} KNOWN}} ( x , y , z , t ) , 2 \\ \\ \\ \\ \vec{{B}_{ACT \text{-} KNOWN}} ( x , y , z , t ) , N\end{matrix} ] = [ \begin{matrix} & & & & \\ & & & & \\ & & & & \\ & & R & & \\ & & & & \\ & & & & \\ & & & & \end{matrix} ] \vec{J} ( t ) . \qquad [ 5 ]$$

In this particular embodiment, the minute contribution of the MEG magnetic field B_{MEG} is ignored for now for purposes of simplicity, such that the environmental magnetic field B_{ENV}, generic environmental magnetic field model B_{ENV-MOD} Maxwell-constrained environmental magnetic field model B_{ENV-MAXWELL}, and parameterized environmental magnetic field model B_{ENV-MAXWELL} can be respectively replaced with the outside magnetic field B_{OUT}, a generic outside magnetic field model B_{OUT-MOD}, a Maxwell-constrained outside magnetic field model B_{OUT-MAXWELL}, and a parameterized outside magnetic field model B_{OUT-MAXWELL}. In this case, the physical portion of the MEG magnetic field B_{MEG} component in the measured total residual magnetic field B_{TOT-MEAS} and the non-physical (error) portion of the MEG magnetic field B_{MEG} component in the measured total residual magnetic field B_{TOT-MEAS} are not distinguished. Rather, only the measurement errors associated with the outside magnetic field B_{OUT} component in the total residual magnetic field measurements B_{TOT-MEAS} will be reduced, such that the outside magnetic field B_{OUT} may be more accurately cancelled, thereby more effectively suppressing the total residual magnetic field B_{TOT} at the fine magnetometers 26b to bring the fine magnetometers 26b in-range.

As briefly discussed above, the processor 30 may generate the environmental magnetic field model B_{ENV-MOD}, and in this particular case the outside magnetic field model B_{OUT-MOD}, in the vicinity of the magnetometers 26. In particular, on the length scale of the signal acquisition unit 18, the outside magnetic field B_{OUT} may assume to have certain physical properties. The processor 30 may generate the generic outside magnetic field model B_{OUT-MOD} in the vicinity of the magnetometers 26 based on these assumed physical properties in any one of a variety of manners, but in the illustrated embodiment, the processor 30 models the outside magnetic field B_{OUT} as a function of space by employing one or more basis functions. In one embodiment, the processor 30 models the outside magnetic field B_{OUT} by employing basis functions having a linear spatial dependence. For example, one basis function may have a uniform (0^{th} order) components and linear (first order) spatial components (i.e., the slope). Second order non-linear spatial components can be ignored, although in alternative embodiments, basis functions with non-linear spatial dependence, or other types of modeling that one of ordinary skill in the art of signal processing, system identification, or control will recognize will serve the same purpose (such as other types of modes or bases, including singular values, eigenvectors, or bases collected from data such as collected by proper orthogonal decomposition or by other fitting methods).

Assuming that the outside magnetic field B_{OUT} can be modeled with only 0^{th} order and 1^{st} order components, a time-varying and spatially-varying generic model of the magnetic field model \vec{B_{OUT-MOD}}(x, y, z, t) is:

$$\vec{{B}_{OUT \text{-} MOD}} ( x , y , z , t ) = [ \begin{matrix}{Bx}_{OUT \text{-} MOD} ( x , y , z , t ) \\ {By}_{OUT \text{-} MOD} ( x , y , z , t ) \\ {Bz}_{OUT \text{-} MOD} ( x , y , z , t )\end{matrix} ] = [ \begin{matrix}{α}_{x} ( t ) + {α}_{xx} ( t ) x + {α}_{xy} ( t ) y + {α}_{xz} ( t ) z \\ {α}_{y} ( t ) + {α}_{yx} ( t ) x + {α}_{yy} ( t ) y + {α}_{yz} ( t ) z \\ {α}_{z} ( t ) + {α}_{zx} ( t ) x + {α}_{zy} ( t ) y + {α}_{zz} ( t ) z\end{matrix} ] + O ( {‖ x , y , z ‖}^{2} ) , \qquad [ 6 ]$$

where O(∥x,y,z∥²) means that the neglected higher order terms produce an error that scales as ∥x,y,z∥², which is the size of the vector to the second power. As described above, this error is practically small for the outside magnetic field B_{OUT}. Hence, the x-directional component Bx_{OUT-MOD}(x, y, z, t) of the magnetic field model \vec{B_{OUT-MOD}}(x, y, z, t) has a 0^{th} order component that is characterized by the time-varying basis function αₓ(t) and 1^{st} order spatial components that linearly vary in the space (x, y, and z) and are respectively characterized by time varying basis functions αₓₓ(t)x, α_{xy}(t)y, and α_{xz}(t)z; the y-directional component By_{OUT-MOD}(x, y, z, t) of the magnetic field model \vec{B_{OUT-MOD}}(x, y, z, t) has a 0^{th} order component that is characterized by the time-varying basis function α_{y}(t) and 1^{st} order spatial components that linearly vary in the space (x, y, and z) and are respectively characterized by time varying basis functions α_{yx}(t)x, α_{yy}(t)y, and α_{yz}(t)z; and the y-directional component Bz_{OUT-MOD}(x, y, z, t) of the magnetic field model \vec{B_{OUT-MOD}}(x, y, z, t) has a 0^{th} order component that is characterized by the time-varying basis function α_{z}(t) and 1^{st} order spatial components that linearly vary in the space (x, y, and z) and are respectively characterized by time varying basis functions α_{zx}(t)x, α_{zy}(t)y, and α_{zz}(t)z.

Thus, a total of 12 initial basis functions (i.e., αₓ(t), αₓₓ(t)x, α_{xy}(t)y, α_{xz}(t)z, α_{y}(t), α_{yx}(t)x, α_{yy}(t)y, α_{yz}(t)z, α_{z}(t), α_{zx}(t)x, α_{zy}(t)y, α_{zz}(t)z) characterizes the magnetic field model \vec{B_{OUT-MOD}}(x, y, z, t). As will be described in further detail below, a coefficient vector [symbol] (t)=[γ₁(t), γ₂ (t), . . . γ₁₂ (t)] respectively associated with these basis functions can be estimated based on the total residual magnetic field measurements \vec{B_{TOT-MEAS}}(x, y, z, t) acquired from the magnetometers 26. Higher order spatial components, such as second order terms in space like x², xy, and z², and third, fourth, and fifth order terms, etc., for this exemplary instance are assumed negligible.

As briefly discussed above, the processor 30 may constrain the environmental magnetic field model B_{MEG-MOD} to generate a Maxwell-constrained environmental magnetic field model B_{MEG-MAXWELL} that satisfies Maxwell's equations, and in this particular case, may constrain the outside magnetic field model \vec{B_{OUT-MOD}}(x, y, z, t) to generate a Maxwell-constrained environmental magnetic field model \vec{B_{OUT-MAXWELL}}(x, y, z, t) that satisfies Maxwell's equations. In particular, using the known physicals of Maxwell's equations, the processor 30 reduce the number of coefficients to be estimated. As a result, a smaller number of coefficients are estimated with the same number of available measurements, and the resulting accuracy of the estimation can improve for two reasons: firstly, because from the total residual magnetic field measurements \vec{B_{TOT-MEAS}}(x, y, z, t) that are available less coefficients need to be estimated; and secondly because exploiting Maxwell's equations as disclosed can allow measurement errors that reflect a situation that is not physically possible (does not satisfy Maxwell's equations) to be eliminated, and thus only the errors (e.g., errors in the generic outside magnetic field model \vec{B_{TOT-MOD}}(x, y, z, t)) that satisfy Maxwell's equations remain. Specifically, the outside magnetic field \vec{B_{OUT}}(x, y, z, t) (or any magnetic field) can be expressed as:

\vec{B_{OUT}}(x,y,z,t)=\vec{B_{PHYSICAL}}(x,y,z,t)+\vec{B_{NON-PHYSICAL}}((x,y,z,t), [7]

where
\vec{B_{PHYSICAL}}(x, y, z, t) satisfies Maxwell's equations and can occur, and
\vec{B_{NON-PHYSICAL}}(x, y, z, t) does not satisfy Maxwell's equations and cannot occur.
Measurement errors can occur in all directions, and can have modes both along \vec{B_{PHYSICAL}}(x, y, z, t) and \vec{B_{NON-PHYSICAL}}(x, y, z, t). Using Maxwell's equations, the processor 30 may distinguish the modes of the outside magnetic field \vec{B_{OUT}}(x, y, z, t) that are physically possible, and the modes of the outside magnetic field \vec{B_{OUT}}(x, y, z, t) that are physically impossible. Thus, employing Maxwell's equations to the generic outside magnetic field model \vec{B_{OUT-MOD}}(x, y, z, t) eliminate errors along the physically not possible direction.

Maxwell's equations include Gauss' Law, Gauss' Law for Magnetism, Faraday's Law, and Ampere's-Maxwell's Law.

Gauss' Law describes the relationship between a static electric field and the electric charges, and in particular, states that the net outflow of the electrical field through any closed surface is proportional to the charge enclosed by the surface, in accordance with:

$$∇ \cdot E = \frac{ρ}{{ϵ}_{0}} , \qquad [ 8 a ]$$

where “∇·” is a divergence operator, E is the electric field, ρ is the total charge per unit volume, and ∈₀ is the permittivity of free space.

Gauss's Law for Magnetism states that there are no magnetic charges (also called “magnetic monopoles”), but instead the magnetic field is generated by a dipole, such that the net outflow of the magnetic field through any closed surface is zero, in accordance with:

∇·B=0, [8b]

where “∇·” is a divergence operator and B is the magnetic field.

Faraday's Law describes the relationship between a time-varying magnetic field and an electric field, and states that, the work per unit charge required to move a charge around a closed loop equals the rate of change of the magnetic flux through the enclosed surface, in accordance with:

$$∇ \times E = - \frac{\partial B}{\partial t} , \qquad [ 8 c ]$$

where “∇×” is the curl operator, E is the electric field, and

$$\frac{\partial B}{\partial L}$$

is the change in magnetic field per unit time.

Ampere's-Maxwell's Law states that magnetic fields can be generated by changing electric fields, and states that the magnetic field induced around any closed loop is proportional to the electric current and the displacement current (proportional to the rate of change of electric flux) through the enclosed surface, in accordance with:

$$∇ \times B = {μ}_{0} {ϵ}_{0} \frac{\partial E}{\partial L} + {μ}_{0} J , \qquad [ 8 d ]$$

where “∇×” is the curl operator, B is the magnetic field,

$$\frac{\partial E}{\partial t}$$

is the change in electric field per unit time, and J is the current density, μ₀ is the permeability of free space, and ∈₀ is the permittivity of free space.

The four Maxwell's equations can be used to constrain the 1st order coefficients αₓₓ, α_{yy}, and α_{zz} to:

αₓₓ+α_{yy}+α_{zz}=0; and [9a]

the remaining 1^{st} order coefficients α_{xy}, α_{yx}, α_{xz}, α_{zx}, α_{yz}, and α_{zy}, (assuming electromagnetic terms are small for the measurement of the frequencies of interest) to:

−α_{xy}+α_{yx}=0; [9b]

α_{xz}−α_{zx}=0; and [9c]

−α_{yz}+α_{zy}=0. [9d]

In total, these are four equations (equations [9a]-[9d]) for 12 coefficients, which can be represented in matrix form as:

M [symbol] (t)=0, [10]

where [symbol] =[αₓ,α_{y}, . . . α_{zz}].
After solving equations [9a]-[9d], there will be 8 degrees of freedom left. Indeed, the null space of the matrix M yields all possible coefficients at any time, by the equation:

[symbol]

(t)=Γ [symbol] , [11]

where [symbol] =[γ₁, γ₂, . . . γ₈] contains only 8 free coefficients instead of 12 free coefficients, and F is a null matrix of the matrix M. Thus, in an exemplary embodiment, there are 8 free parameters that reduces the generic outside magnetic field model \vec{B_{OUT-MOD}}(x, y, z, t) of equation [6] to a Maxwell-constrained outside magnetic field model \vec{B_{OUT-MAXWELL}}(x, y, z, t) with eight basis functions corresponding to the Maxwell-constrained coefficient vector [symbol] (t)=[γ₁(t), γ₂(t), . . . γ₈(t)]. The Maxwell-constrained outside magnetic field model \vec{B_{OUT-MAXWELL}}(x, y, z, t) at the magnetometers 26 can be represented by a matrix of influence Q from the Maxwell-constrained coefficient vector [symbol] (t)=[γ₁(t), γ₂ (t), . . . γ₈ (t)] to the Maxwell-constrained outside magnetic field model \vec{B_{OUT-MAXWELL}}(x, y, z, t) at the N number of magnetometers 26. Thus, the generic outside magnetic field model \vec{B_{OUT-MAXWELL}}(x, y, z t) at the N number of magnetometers 26 may be given as:

$$[ \begin{matrix}\vec{{B}_{OUT \text{-} MAXWELL}} ( x , y , z , t ) , 1 \\ \vec{{B}_{OUT \text{-} MAXWELL}} ( x , y , z , t ) , 2 \\ \\ \\ \\ \vec{{B}_{OUT \text{-} MAXWELL}} ( x , y , z , t ) , N\end{matrix} ] = [ \begin{matrix} & & & & \\ & & & & \\ & & & & \\ & & Q & & \\ & & & & \\ & & & & \\ & & & & \end{matrix} ] \vec{γ} ( t ) . \qquad [ 12 ]$$

As briefly discussed above, the processor 30 may parameterize the Maxwell-constrained environmental magnetic field model B_{ENV-PAR} based on the measured total residual magnetic field B_{TOT-MEAS} measured by the magnetometers 26 and the known actuated magnetic field B_{ACT-KNOWN} at the magnetometers 26 to generate a parameterized environmental magnetic field model B_{ENV-PAR}, and in this particular, case, may parameterize the Maxwell-constrained outside magnetic field model \vec{B_{OUT-MAXWELL}}(x, y, z, t) based on the total residual magnetic field \vec{B_{TOT-MEAS}}(x, y, z, t) measured by the magnetometers 26 and the known actuated magnetic field \vec{B_{ACT-KNOWN}}(x, y, z, t) at the magnetometers 26 to generate a parameterized outside magnetic field model B_{OUT-PAR}.

In particular, assuming that the very weak MEG magnetic field B_{MEG} can be ignored for purposes of simplicity, it is known that the following equation holds true at each of the magnetometers 26:

$$[ \begin{matrix}\vec{{B}_{TOT}} ( x , y , z , t ) , 1 \\ \vec{{B}_{TOT}} ( x , y , z , t ) , 2 \\ \\ \\ \\ \vec{{B}_{TOT}} ( x , y , z , t ) , N\end{matrix} ] = [ \begin{matrix}\vec{{B}_{ACT}} ( x , y , z , t ) , 1 \\ \vec{{B}_{ACT}} ( x , y , z , t ) , 2 \\ \\ \\ \\ \vec{{B}_{ACT}} ( x , y , z , t ) , N\end{matrix} ] + [ \begin{matrix}\vec{{B}_{OUT}} ( x , y , z , t ) , 1 \\ \vec{{B}_{OUT}} ( x , y , z , t ) , 2 \\ \\ \\ \\ \vec{{B}_{OUT}} ( x , y , z , t ) , N\end{matrix} ] , \qquad [ 1 ]$$

where \vec{B_{TOT}}(x, y, z, t) is the true total magnetic field measurement at the magnetometers 26, \vec{B_{ACT}}(x, y, z, t) is the true actuated magnetic field at the magnetometers 26, and \vec{B_{OUT}}(x, y, z, t) is the true outside magnetic field at the magnetometers 26.

Substituting the total residual magnetic field measurements \vec{B_{TOT-MEAS}}(x, y, z, t) at the magnetometers 26 of the term [1] for the true total residual magnetic field \vec{B_{TOT}}(x, y, z, t) at the magnetometers 26 of equation [13], the known actuated magnetic field \vec{B_{ACT-KNOWN}}(x, y, z, t) at the magnetometers 26 of equation [5] for the true actuated magnetic field \vec{B_{ACT}}(x, y, z, t) at the magnetometers 26 of equation [13], and the Maxwell-constrained outside magnetic field model \vec{B_{OUT-MAXWELL}}(x, y, z, t) at the magnetometers 26 of equation [12] for the true outside magnetic field \vec{B_{OUT}}(x, y, z, t) at the magnetometers 26 of equation [13] yields:

$$[ \begin{matrix}\vec{{B}_{TOT \text{-} MEAS}} ( x , y , z , t ) , 1 \\ \vec{{B}_{TOT \text{-} MEAS}} ( x , y , z , t ) , 2 \\ \\ \\ \\ \vec{{B}_{TOT \text{-} MEAS}} ( x , y , z , t ) , N\end{matrix} ] = [ \begin{matrix} & & & & \\ & & & & \\ & & & & \\ & & R & & \\ & & & & \\ & & & & \\ & & & & \end{matrix} ] \vec{J} ( t ) + [ \begin{matrix} & & & & \\ & & & & \\ & & & & \\ & & Q & & \\ & & & & \\ & & & & \\ & & & & \end{matrix} ] \vec{γ} ( t ) + [ \begin{matrix}{δ}_{1} \\ {δ}_{2} \\ \\ \\ \\ \\ {δ}_{N}\end{matrix} ] , \qquad [ 14 ]$$

where δ is unknown measurement noise for each magnetometer 26.

The processor 30 may employ any suitable fitting optimization technique (including linear and nonlinear methods, gradient descent, matrix methods, system identification, or machine learning methods, etc.) to fit the Maxwell-constrained coefficient vector [symbol] (t) of the Maxwell-constrained outside magnetic field model \vec{B_{OUT-MAXWELL}}(x, y, z, t) to the difference between the total residual magnetic field \vec{B_{TOT-MEAS}}(x, y, z, t) measured by the magnetometers 26 and the known actuated magnetic field \vec{B_{ACT-KNOWN}}(x, y, z, t) at the magnetometers 26. In the illustrated embodiment, the processor 30 employs a least squares or weighted least squares optimization technique, which serves to minimize the error between collected and known data and estimated data, to accurately estimate the values of the Maxwell-constrained coefficient vectors [symbol] (t) of the Maxwell-constrained outside magnetic field model \vec{B_{OUT-MAXWELL}}(x, y, z, t) at the magnetometers 26. That is, the solution that minimizes the difference between the total residual magnetic field \vec{B_{TOT-MEAS}}(x, y, z, t) measured by each of the magnetometers 26 and the product of the matrix of influence R at the magnetometers 26 and the vector of actuation strengths [symbol] (t) of the set of magnetic field actuators 28 yields an estimate of the Maxwell-constrained coefficient vector [symbol] (t) of the Maxwell-constrained outside magnetic field model \vec{B_{OUT-MAXWELL}}(x, y, z, t) at the magnetometers 26.

Specifically, the least squares estimate of the Maxwell-constrained coefficient vector \vec{γ*}(t) of the Maxwell-constrained outside magnetic field model \vec{B_{OUT-MAXWELL}}(x, y, z, t) can be provided as:

\vec{γ*}(t)=[Q^{T}Q]⁻¹Q^{T}(B_{TOT-MEAS}(t)−R* [symbol] (t)), [15]

where Q is the matrix of influence from the Maxwell-constrained coefficient vector [symbol] (t)=[γ₁(t), γ₂ (t), . . . γ₈(t)] to the Maxwell-constrained outside magnetic field model \vec{B_{OUT-MAXWELL}}(x, y, z, t) at the N number of magnetometers 26; B_{TOT-MEAS}(t) is the time-varying matrix of total residual magnetic field measurements \vec{B_{TOT-MEAS}}(x, y, z, t) at the N number of magnetometers 26; [symbol] (t) is the actuation strength vector; R is the matrix of influence from the actuation strength vector [symbol] (t) to the known actuated magnetic field \vec{B_{ACT-KNOWN}}(x, y, z, t) at the N number of magnetometers 26; the superscript T denotes the matrix transpose; and the superscript −1 denotes matrix inversion.

A parameterized outside magnetic field model \vec{B_{OUT-PAR}}(x, y, z, t) may be generated by substituting the solved Maxwell-constrained coefficient vector \vec{γ*}(t) into equation [6]. It should be appreciated that the foregoing method transforms a discrete set of the total residual magnetic field measurements \vec{B_{TOT-MEAS}}(x, y, z, t) into continuous parameterizations of the outside magnetic field \vec{B_{OUT}}(x, y, z, t), i.e., the parameterized outside magnetic field model \vec{B_{OUT-PAR}}(x, y, z, t). This enables the processor 30 to estimate the outside magnetic field B_{OUT} at arbitrary locations in the vicinity from which the measurements of the total residual magnetic field B_{TOT-MEAS} were acquired, i.e., in the vicinity of the signal acquisition unit 18.

As briefly discussed above, the processor 30 may determine the environmental magnetic field estimates B_{ENV-EST} at the magnetometers 26 based on the parameterized environmental magnetic field model B_{ENV-PAR}, and in this particular case, may determine the outside magnetic field estimates \vec{B_{OUT-EST}}(x, y, z, t) at the magnetometers 26 based on the parameterized outside magnetic field model \vec{B_{OUT-PAR}}(x, y, z, t).

In particular, the outside magnetic field estimates \vec{B_{OUT-EST}}(x, y, z, t) at the magnetometers 26 may be determined by substituting the (x,y,z) locations of the magnetometers 26 into the parameterized outside magnetic field model \vec{B_{OUT-PAR}}(x, y, z, t); i.e., the outside magnetic field estimates \vec{B_{OUT-EST}}(x, y, z, t) at the magnetometers 26 may be recovered from the product of the influence matrix Q and the least squares fit values of the Maxwell-constrained coefficient vector \vec{γ*}(t).

As briefly discussed above, the processor 30 may determine the total residual magnetic field estimates B_{TOT-EST} at the magnetometers 26 based on the known actuated magnetic field B_{ACT-KNOWN} at the magnetometers 26 and the environmental magnetic field estimates B_{OUT-EST} and in this particular case, may determine the total residual magnetic field estimates \vec{B_{TOT-EST}}(x, y, z, t) at magnetometers 26 based on the known actuated magnetic field \vec{B_{ACT-KNOWN}}(x, y, z, t) at the magnetometers 26 and the outside magnetic field estimates \vec{B_{OUT-EST}}(x, y, z, t), at the magnetometers 26 by summing the known actuated magnetic field \vec{B_{ACT-KNOWN}}(x, y, z, t) at the magnetometers 26 and the outside magnetic field estimates \vec{B_{OUT-EST}}(x, y, z, t) at the magnetometers 26.

In particular, substituting the total residual magnetic field estimates \vec{B_{TOT-EST}}(x, y, z, t) at the magnetometers 26 for the true total residual magnetic field \vec{B_{TOT}}(x, y, z, t) at the magnetometers 26 of equation [13], the known actuated magnetic field \vec{B_{ACT-KNOWN}}(x, y, z, t) at the magnetometers 26 of equation [5] for the true actuated magnetic field \vec{B_{ACT}}(x, y, z, t) at the magnetometers 26 of equation [13], and the outside magnetic field estimates \vec{B_{OUT-EST}}(x, y, z, t) at the magnetometers 26 for the true outside magnetic field \vec{B_{OUT}}(x, y, z, t) at the magnetometers 26 of equation [13] yields:

$$[ \begin{matrix}\vec{{B}_{TOT \text{-} EST}} ( x , y , z , t ) , 1 \\ \vec{{B}_{TOT \text{-} EST}} ( x , y , z , t ) , 2 \\ \\ \\ \\ \vec{{B}_{TOT \text{-} EST}} ( x , y , z , t ) , N\end{matrix} ] = [ \begin{matrix}\vec{{B}_{ACT \text{-} KNOWN}} ( x , y , z , t ) , 1 \\ \vec{{B}_{ACT \text{-} KNOWN}} ( x , y , z , t ) , 2 \\ \\ \\ \\ \vec{{B}_{ACT \text{-} KNOWN}} ( x , y , z , t ) , N\end{matrix} ] + [ \begin{matrix}\vec{{B}_{OUT \text{-} EST}} ( x , y , z , t ) , 1 \\ \vec{{B}_{OUT \text{-} EST}} ( x , y , z , t ) , 2 \\ \\ \\ \\ \vec{{B}_{OUT \text{-} EST}} ( x , y , z , t ) , N\end{matrix} ] , \qquad [ 16 ]$$

Thus, intuitively, equation [16] need only be solved to accurately infer the total residual magnetic field estimates B_{TOT-EST} at the magnetometers 26.

It can be appreciated that inferring the total residual magnetic field estimates \vec{B_{TOT-EST}}(x, y, z, t) at that magnetometers 26 based on the total residual magnetic field measurements \vec{B_{TOT-MEAS}}(x, y, z, t) taken by all of the magnetometers 26 (including the magnetometer 26 for which the total residual magnetic field estimates \vec{B_{TOT-EST}}(x, y, z, t) is being inferred) provides a more accurate assessment of the true total residual magnetic field \vec{B_{TOT}}(x, y, z, t) at each magnetometer 26 than each magnetometer 26 can measure alone, because such inference technique averages out the unknown measurement noise δ of the magnetometers 26 in a rigorous manner. Thus, in effect, the total residual magnetic field measurement \vec{B_{TOT-MEAS}}(x, y, z, t) taken by each magnetometer 26 is corrected by this inference technique.

Further, as a result of generating a Maxwell-constrained outside magnetic field model B_{OUT-MAXWELL} by applying Maxwell's equations to the generic outside magnetic field model B_{OUT-MOD}, the non-physical portion of the total residual magnetic field measurement \vec{B_{TOT-MEAS}}(x, y, z, t) taken by each magnetometer 26, and in this particular case, the outside magnetic field B_{OUT} component of the total residual magnetic field measurement \vec{B_{TOT-MEAS}}(x, y, z, t), is eliminated in the total residual magnetic field estimates \vec{B_{TOT-EST}}(x, y, z, t) at that magnetometers 26, or at the least, the non-physical portion in the total residual magnetic field estimates \vec{B_{TOT-EST}}(x, y, z, t) at the magnetometers 26 will be substantially less than the non-physical portion in the total residual magnetic field measurements \vec{B_{TOT-EST}}(x, y, z, t) at that magnetometers 26. As a result, the processor 30 may control the actuated magnetic field \vec{B_{ACT}}(x, y, z, t) in a manner that more accurately cancels the outside magnetic field \vec{B_{OUT}}(x, y, z, t), such that the total residual magnetic field \vec{B_{TOT}}(x, y, z, t) at the magnetometers 26 can be more effectively suppressed.

While acquiring total residual magnetic field measurements \vec{B_{TOT-MEAS}}(x, y, z, t) from the magnetometers 26 and inferring the total residual magnetic field estimates \vec{B_{TOT-EST}}(x, y, z, t) at the magnetometers 26 can be conducted over one time or over all available time, it is preferred that it be conducted over a time window that is updated in time (e.g. from current time t back till time t−T, where T is the time window period and can be constant or variable), since doing so over a longer time period allows the unknown measurement noise δ of the magnetometers 26 to be averaged out.

In one embodiment, the gain and offset of each of the coarse magnetometers 26a can be estimated by comparing the more accurate total residual magnetic field estimate \vec{B_{TOT-EST}}(x, y, z, t) at each coarse magnetometer 26a that has been inferred from total residual magnetic field measurements \vec{B_{TOT-MEAS}}(x, y, z, t) at many magnetometers 26 (including the much more accurate fine magnetometers 26b) to the total residual magnetic field \vec{B_{TOT-MEAS}}(x, y, z, t) measured by the respective coarse magnetometer 26a.

In another embodiment, a weighted least squares estimate, instead of an unweighted least squares estimate, of the Maxwell-constrained coefficient vector \vec{γ*}(t) of the Maxwell-constrained outside magnetic field model \vec{B_{OUT-MAXWELL}}(x, y, z, t) is employed. For example, total residual magnetic field measurements \vec{B_{TOT-MEAS}^{F}}(x, y, z, t) acquired from fine magnetometers 26b are substantially more accurate than total residual magnetic field measurements \vec{B_{TOT-MEAS}^{C}}(x, y, z, t) acquired from coarse magnetometers 26a. Furthermore, due to drifts in the offset or gain of coarse magnetometers 26a over time, newer total residual magnetic field measurements \vec{B_{TOT-MEAS}^{C}}(x, y, z, t) acquired from coarse magnetometers 26b, absent re-calibration, are more accurate than older total residual magnetic field measurements \vec{B_{TOT-MEAS}}(x, y, z, t) acquired from the same coarse magnetometers 26b.

As discussed above, acquiring total residual magnetic field measurements \vec{B_{TOT-MEAS}}(x, y, z, t) from the magnetometers 26 and inferring the total residual magnetic field estimates \vec{B_{TOT-EST}}(x, y, z, t) at the magnetometers 26 is preferably conductive over a time window that is updated in time (e.g. from current time t back till time t−T, where T is the time window period and can be constant or variable), since doing so over a longer time period allows the unknown measurement noise δ of the magnetometers 26 to be averaged out.

Thus, in a preferred embodiment, total residual magnetic field measurements \vec{B_{TOT-MEAS}}(x, y, z, t) are acquired from the magnetometers 26 and the total residual magnetic field estimates \vec{B_{TOT-EST}}(x, y, z, t) are inferred at the magnetometers 26 over an updated time window, the total residual magnetic field measurements \vec{B_{TOT-MEAS}^{F}}(x, y, z, t) acquired from fine magnetometers 26b are weighed higher than total residual magnetic field measurements \vec{B_{TOT-MEAS}^{C}}(x, y, z, t) acquired from coarse magnetometers 26a, and newer total residual magnetic field measurements \vec{B_{TOT-MEAS}^{F}}(x, y, z, t) acquired from magnetometers 26 are weighted higher than older total residual magnetic field measurements \vec{B_{TOT-MEAS}}(x, y, z, t) acquired from magnetometers 26.

Such weighting can be incorporated into a weighting matrix W that operates on all the total residual magnetic field measurements \vec{B_{TOT-MEAS}}(x, y, z, t) acquired from magnetometers 26. Elements of the weighting matrix W can be selected to be inversely proportional to the measurement variance of each magnetometer 26, such that total residual magnetic field measurements \vec{B_{TOT-MEAS}^{C}}(x, y, z, t) acquired from coarse magnetometers 26a (which have a high measurement variance (and thus relatively low accuracy) have a small weight, while total residual magnetic field measurements \vec{B_{TOT-MEAS}^{F}}(x, y, z, t) acquired from fine magnetometers 26b (which have a low measurement variance (and thus relatively high accuracy) have a large weight. Furthermore, elements of the weighting matrix W may decrease as the total residual magnetic field measurements \vec{B_{TOT-MEAS}}(x, y, z, t) acquired from magnetometers 26 age, such that older total residual magnetic field measurements \vec{B_{TOT-MEAS}}(x, y, z, t) have a small weight and newer total residual magnetic field measurements \vec{B_{TOT-MEAS}}(x, y, z, t) have a large weight. This decrease in the elements of the weighting matrix W may be linear, quadratic, stepwise, or have some other functional dependence that can be selected by intuition or by mathematical optimization by methods known to those of ordinary skill in the art of optimization, control and signal processing, or system identification. In one embodiment, the functional dependence may match the time scale of how quickly the gain or offset of the coarse magnetometers drift in time.

The unweighted least squares estimate of equation [15] can then be modified with the weighting matrix W as:

\vec{γ*}((t−T)→t)=[Q^{T}W^{T}WQ]⁻¹Q^{T}W^{T}W(B_{TOT-MEAS}((t−T)→t)−R* [symbol] ((t−T)→t)), [17]

where Q is the matrix of influence from the Maxwell-constrained coefficient vector [symbol] (t)=[γ₁(t), γ₂(t), . . . γ₈ (t)] to the Maxwell-constrained outside magnetic field model \vec{B_{OUT-MAXWELL}}(x, y, z, t) at the N number of magnetometers 26; ((t−T)→t) is an updated time window; B_{TOT-MEAS}((t−T)→t) is the time-varying matrix of the total residual magnetic field measurements \vec{B_{TOT-MEAS}}(x, y, z, t) at the N number of magnetometers 26 over the time window ((t−T)→t); [symbol] ((t−T)→t) is the time-varying vector of actuation strengths over the time window ((t−T)→t); R is the matrix of influence from the actuation strength vector [symbol] ((t−T)→t) to the known actuated magnetic field \vec{B_{ACT-KNOWN}}(x, y, z, t) at the N number of magnetometers 26; the superscript T denotes the matrix transpose; the superscript −1 denotes matrix inversion; and W is the weighting matrix.

Although all three of the directional components of the outside magnetic field B_{OUT} and actuated magnetic field B_{ACT}, and thus all three of the directional components of the total residual magnetic field B_{TOT}, have been considered when inferring the total residual magnetic field estimates B_{TOT-EST} at the magnetometers 26, it should be appreciated that only one or two directional components of the outside magnetic field B_{OUT} and/or actuated magnetic field B_{ACT} may be considered when inferring the total residual magnetic field estimates B_{TOT-EST} at the magnetometers 26. Furthermore, although all three directional components of the total residual magnetic field B_{TOT-MEAS} have been described as being measured and estimated at the same location or virtually at the same location for each magnetometer 26, less than three directional components of the total residual magnetic field B_{TOT-MEAS} may be measured and/or estimated at the same location or virtually at the same location for each magnetometer 26.

As discussed above, Maxwell's equations can be applied to the total residual magnetic field measurements \vec{B_{TOT-MEAS}}(x, y, z, t) to eliminate, or at least substantially reduce, the non-physical portion of the MEG magnetic field \vec{B_{MEG}}(x, y, z, t) component of the total residual magnetic field measurements \vec{B_{TOT-MEAS}}(x, y, z, t) such that the MEG magnetic field \vec{B_{MEG}}(x, y, z, t) may be more accurately determined. The non-physical portion of the MEG magnetic field \vec{B_{MEG}}(x, y, z, t) component of the total residual magnetic field measurements \vec{B_{TOT-MEAS}}(x, y, z, t) can be reduced by performing the same procedure used to reduce the non-physical portion of the MEG magnetic field \vec{B_{MEG}}(x, y, z, t) component of the total residual magnetic field measurements \vec{B_{TOT-MEAS}}(x, y, z, t) described above, with the exception that a generic model of the environmental magnetic field \vec{B_{ENV-MOD}}(x, y, z, t) containing both initial basis functions (e.g., the 0^{th} order and 1^{st} order basis functions) for the \vec{B_{OUT}}(x, y, z, t) and initial basis functions for the MEG magnetic field \vec{B_{MEG}}(x, y, z, t).

Thus, the processor 30 may be configured for inferring total residual magnetic field estimates \vec{B_{TOT-EST}}(x, y, z, t) at the magnetometers 26 by (1) acquiring the measurements of the total residual magnetic field \vec{B_{TOT-MEAS}}(x, y, z, t) from the magnetometers 26, as exemplified by the previous discussion related to equations [1] and [2]; (2) determining the known actuated magnetic field \vec{B_{ACT-KNOWN}}(x, y, z, t) at the magnetometers 26 based on a known profile of the set of magnetic field actuators 28 and the actuation strengths of the magnetic field actuators 28, as exemplified by previous discussion relating to equations [3]-[5]; (3) generating a generic model of the environmental magnetic field \vec{B_{ENV-MOD}}(x, y, z, t) in the vicinity of the magnetometers 26 in a similar manner that the generic outside magnetic model \vec{B_{OUT-MOD}}(x, y, z, t) is generated in the discussion related to equation [6] with the exception that the generic environmental magnetic field model \vec{B_{ENV-MOD}}(x, y, z, t) comprises additional initial basis functions for the MEG magnetic field \vec{B_{MEG}}(x, y, z, t); (4) constraining the environmental magnetic field model \vec{B_{ENV-MOD}}(x, y, z, t) to generate a Maxwell-constrained model of the environmental magnetic field \vec{B_{ENV-MAXWELL}}(x, y, z, t) that satisfies Maxwell's equations, in a similar manner that generic outside magnetic model \vec{B_{OUT-MOD}}(x, y, z, t) is constrained in the discussion related to equations [7]-[12], with the exception that the initial basis functions for the outside magnetic field \vec{B_{OUT-MOD}}(x, y, z, t) and the MEG magnetic field \vec{B_{MEG}}(x, y, z, t) are collectively reduced; (5) parameterizing the Maxwell-constrained environmental magnetic field model \vec{B_{ENV-MAXWELL}}(x, y, z, t) based on the measured total residual magnetic field \vec{B_{TOT-MEAS}}(x, y, z, t) measured by the magnetometers 26 and the known actuated magnetic field \vec{B_{ACT-KNOWN}}(x, y, z, t) at the magnetometers 26 to generate a parameterized environmental magnetic field model \vec{B_{ENV-ENV}}(x, y, z, t), in a similar manner that the \vec{B_{OUT-MAXWELL}}(x, y, z, t) is constrained in the discussion related to equations [13]-[15]; (6) determining the environmental magnetic field estimates \vec{B_{ENV-EST}}(x, y, z, t) at the magnetometers 26 based on the parameterized environmental magnetic field model \vec{B_{ENV-ENV}}(x, y, z, t) by substituting the locations of the magnetometers 26 into the parameterized environmental magnetic field model \vec{B_{ENV-ENV}}(x, y, z, t); and (7) determining the total residual magnetic field estimates \vec{B_{TOT-EST}}(x, y, z, t) at the magnetometers 26 based on the known actuated magnetic field \vec{B_{ACT-KNOWN}}(x, y, z, t) at the magnetometers 26 and the environmental magnetic field estimates \vec{B_{ENV-EST}}(x, y, z, t) at the magnetometers 26, in the same manner as the total residual magnetic field estimates \vec{B_{TOT-EST}}(x, y, z, t) is determined in the discussion related to equation [16].

In another embodiment, the processor 30 may be configured for more accurately estimating a magnetic field component of measurements of an arbitrary magnetic field B_{ARB-MEAS} at the magnetometers 26b by (1) generating a generic magnetic field model B_{ARB-MOD} of a plurality of magnetic field components of the arbitrary magnetic field B_{ARB} in the vicinity of the magnetometers 26, with the generic magnetic field model B_{ARB-MOD} comprising a plurality of basis functions having multiple sets of basis functions respectively corresponding to a plurality of magnetic components of the arbitrary magnetic field measurements B_{ARB-MEAS} at the magnetometers 26; (2) parameterizing the generic magnetic field model B_{OUT-MOD} by simultaneously fitting coefficients of the plurality of basis functions at least partially based on the arbitrary magnetic field measurements B_{ARB-MEAS} at the magnetometers 26, thereby yielding a parameterized magnetic field model B_{ARB-PAR} of the magnetic field components of the arbitrary magnetic field B_{ARB} in the vicinity of the magnetometers 26; and estimating one of the magnetic field components of the arbitrary magnetic field B_{ARB} at each of the fine magnetometers 26b based on one of the multiple sets of basis functions of the parameterized magnetic field model B_{ARB-PAR}, and optionally estimating additional ones of the magnetic field components of the arbitrary magnetic field B_{ARB} at each of the fine magnetometers 26b based additional ones of the multiple sets of basis functions of the parameterized magnetic field model B_{ARB-PAR}.

In particular, when the N number of total residual magnetic field measurements \vec{B_{TOT-MEAS}}(x, y, z, t) respectively taken by an N number of magnetometers 26 is greater than a p number of basis functions (i.e., modes) in an arbitrary magnetic field \vec{B_{ARB}}(x, y, z, t) (i.e., p<N), a generic model of the arbitrary magnetic field \vec{B_{ARB-MOD}}(x, y, z, t) containing basis functions corresponding to modes of different magnetic field components in the arbitrary magnetic field \vec{B_{ARB}}(x, y, z, t) may be generated. The generic arbitrary magnetic field model \vec{B_{ARB-MOD}}(x, y, z, t) may be represented as an influence matrix Z from a coefficient vector [symbol] (t) containing p number of coefficients [γ₁(t), γ₂ (t), . . . γₚ (t)] to the arbitrary magnetic field \vec{B_{ARB}}(x, y, z, t) at the N number of magnetometers 26. The influence matrix Z has an N number of row vectors and a p number of column vectors, as follows:

$$Z = [ \begin{matrix}{Z}_{11} & … & {Z}_{1 p} \\ ⋮ & ⋱ & ⋮ \\ {Z}_{N 1} & … & {Z}_{Np}\end{matrix} ] . \qquad [ 18 ]$$

The N number of row vectors correspond to the N number of total residual magnetic field measurements \vec{B_{TOT-MEAS}}(x, y, z, t) respectively taken by the N number of magnetometers 26, while the p number of column vectors respectively correspond to the basis functions (i.e., modes) of the arbitrary magnetic field \vec{B_{ARB}}(x, y, z, t). Significantly, the influence matrix Z contains multiple influence matrices Q_{RETAIN}, Q′_{DISCARD}, Q″_{DISCARD}, . . . , as follows:

Z=[Q_{RETAIN} Q′_{DISCARD} Q″_{DISCARD} . . . ], [19]

where the column vectors of the influence matrix Q_{RETAIN} respectively correspond to the modes of the arbitrary magnetic field \vec{B_{ARB}} (x, y, z, t) to be retained; the column vectors of the influence matrix Q′_{DISCARD} respectively correspond to the modes of the arbitrary magnetic field \vec{B_{ARB}}(x, y, z, t) to be discarded; the column vectors of the influence matrix Q′_{DISCARD} respectively correspond to additional modes of the arbitrary magnetic field \vec{B_{ARB}}(x, y, z, t) to be discarded; and so forth. The column vectors of the influence matrix Q′_{DISCARD} are orthogonal to the column vectors of the influence matrix Q_{RETAIN}, the column vectors of the influence matrix Q″_{DISCARD} are orthogonal to the column vectors of the influence matrices Q_{RETAIN} and Q_{DISCARD}, and so forth. Thus, the influence matrix Z is defined by a concatenation of orthogonal influence matrices Q_{RETAIN}, Q′_{DISCARD}, Q″_{DISCARD}, . . . , with the column vectors to the right of the influence matrix Q_{RETAIN} being considered the rejection space, with the basis functions in the influence.

Although the influence matrix Z is illustrated here as being concatenated with only one influence matrix Q_{RETAIN} to be retained and several influence matrices Q_{DISCARD} to be discarded, it should be appreciated that the influence matrix Z may be concatenated with multiple influence matrices Q_{RETAIN} to be retained with or without one or more influence matrices Q_{DISCARD} to be discarded. Thus, the influence matrix Z may be concatenated with any combination of influence matrices Q_{RETAIN} to be retained and/or influence matrices Q_{DISCARD} to be discarded as long the concatenated influence matrices Q_{RETAIN} and/or Q_{DISCARD} contain mutually exclusive modes of multiple magnetic field components.

The generic arbitrary magnetic field model \vec{B_{ARB-MOD}}(x, y, z, t) may then be parameterized to generate a parameterized model of the arbitrary magnetic field \vec{B_{ARB-PAR}}(x, y, z) by determining the least squares estimate of the coefficient vector \vec{γ*}(t) in the manner discussed above with respect to equation [15] with the exception that the influence matrix Q is replaced with the concatenated influence matrix Z, as follows:

\vec{γ_{RETAIN}*}(t)[Z^{T}Z]⁻¹Z^{T}(\vec{B_{TOT-MEAS}}(x,y,z,t)−R* [symbol] (t)){1:p_{RETAIN}}; [20a]

\vec{γ′_{DISCARD}*}(t)=[Z^{T}Z]⁻¹Z^{T}(\vec{B_{TOT-MEAS}}(x,y,z,t)−R* [symbol] (t)){p_{RETAIN}+1:p_{RETAIN}+p′_{DISCARD}}; [20b]

\vec{y″_{DISCARD}*}(t)=[Z^{T}Z]⁻¹Z^{T}(\vec{B_{TOT-MEAS}}(x,y,z,t)−R* [symbol] (t){p_{RETAIN}+p′_{DISCARD}:p_{RETAIN}+p′_{DISCARD}+p″_{DISCARD}}, and so forth, [20c]

where B_{TOT-MEAS}(x, y, z, t), Z, R, and [symbol] (t) have been defined above; the notation X{A:B} means take the Ath through Bth elements of X; \vec{γ_{RETAIN}*}(t) is the least squares solution of the coefficient vector corresponding to the influence matrix Q to be retained; p_{RETAIN} are the number of modes of the arbitrary magnetic field \vec{B_{ARB}}(x, y, z, t) corresponding to the influence matrix Q to be retained; \vec{γ′_{DISCARD}*}(t) is the least squares solution of the coefficient vector corresponding to the influence matrix Q′ to be discarded; p′_{DISCARD} is the number of modes of the arbitrary magnetic field \vec{B_{ARB}}(x, y, z, t) corresponding to the influence matrix Q′ to be discarded \vec{γ″_{DISCARD}*}(t) is the least squares solution of the coefficient vector corresponding to the influence matrix Q″ to be discarded; p′_{DISCARD} is the number of modes of the arbitrary magnetic field \vec{B_{ARB}} (x, y, z, t) corresponding to the influence matrix Q″ to be discarded, and so forth.

Thus, it can be appreciated that the promotion of a single influence matrix Q to be retained to an influence matrix Z containing both an influence matrix Q to be retained and influence matrices Q′, Q″ . . . to be discarded, in effect, fusing modes of the arbitrary magnetic field \vec{B_{ARB}} (x, y, z, t) derived from multiple models of the arbitrary magnetic field \vec{B_{ARB}}(x, y, z, t), enables separation and precise specification of the modes of the arbitrary magnetic field \vec{B_{ARB}}(x, y, z, t) to be retained and to be discarded. Thus, by simultaneously fitting the coefficient vector \vec{γ_{RETAIN}*}(t) and coefficient vectors \vec{γ′_{DISCARD}*}(t) and \vec{γ″_{DISCARD}*}(t) to the difference between the total residual magnetic field \vec{B_{TOT-MEAS}}(x, y, z, t) measured by the magnetometers 26 and the known actuated magnetic field \vec{B_{ACT-KNOWN}}(x, y, z, t) at the magnetometers 26, the accuracy of the solution for the coefficient vector \vec{γ_{RETAIN}*}(t) is increased.

Arbitrary magnetic field estimates \vec{B_{ARB-EST}}(x, y, z, t) may be determined at the fine magnetometers 26b for any particular magnetic field component of interest by substituting the (x,y,z) locations of the fine magnetometers 26b into the basis functions of the parameterized arbitrary magnetic field model \vec{B_{ARB-PAR}}(x, y, z, t) corresponding to that magnetic field component of interest; i.e., such magnetic field component of the arbitrary magnetic field estimates B_{ARB-EST}(x, y, z, t) at the fine magnetometers 26b may be recovered from the product of the influence matrix Z and the least squares fit values of the coefficient vector \vec{γ*}(t) corresponding to that magnetic field component. For example, one magnetic field component of the arbitrary magnetic field estimates \vec{B_{ARB-EST}}(x, y, z, t) at the fine magnetometers 26 may be recovered from the product of the influence matrix Z and the least squares fit values of the coefficient vector \vec{γ_{RETAIN}*}(t) to be retained. The magnetic field components of the arbitrary magnetic field estimates \vec{B_{ARB-EST}}(x, y, z, t) that are not of interest may simply be ignored, and therefore, not estimated at the fine magnetometers 26b.

In one specific embodiment, the processor 30 may employ the equations [19] and [20a]-[20c] to distinguish between a physical outside magnetic field \vec{B_{OUT-P}}(x, y, z, t) component and a non-physical outside magnetic field \vec{B_{OUT-NP}}(x, y, z, t) component of the total residual magnetic field measurements B_{TOT}(x, y, z, t) acquired from the magnetometers 26. In particular, based on equation [6] above, the generic outside magnetic field model B_{OUT-MOD} (x, y, z, t) can be partitioned into a physically possible magnetic field model \vec{B_{OUT-P-MOD}}(x, y, z, t) that satisfies Maxwell's equations (corresponded to the Maxwell-constrained outside magnetic field model \vec{B_{OUT-MAXWELL}}(x, y, z, t)), and a physically impossible magnetic field \vec{B_{OUT-NP-MOD}}(x, y, z, t) that does not satisfy Maxwell's equations. In this case, the outside magnetic field \vec{B_{OUT-MOD}}(x, y, z, t) has twelve basis functions (i.e., modes), and in particular, αₓ(t), αₓₓ(t)x, α_{xy}(t)y, α_{xz}(t)z, α_{y}(t), α_{yx}(t)x, α_{yy}(t)y, α_{yz}(t)z, α_{z}(t), α_{zx}(t)x, α_{zy}(t)y, α_{zz}(t)z) and a coefficient vector [γ₁(t), γ₂(t), . . . γ₁₂(t)] (i.e., p=12). The physically possible magnetic field model \vec{B_{OUT-P-MOD}}(x, y, z, t) has eight twelve basis functions (i.e., modes) and a coefficient vector [γ₁(t), γ₂(t), . . . γ₈(t)], while physically possible magnetic field model \vec{B_{OUT-P-MOD}}(x, y, z, t) has four twelve basis functions (i.e., modes) and a coefficient vector [γ₉(t), γ₂(t), . . . γ₁₂(t)].

The modes of the physically possible magnetic field model \vec{B_{OUT-P-MOD}}(x, y, z, t) are to be retained, whereas the modes of the physically impossible magnetic field model \vec{B_{OUT-NP-MOD}}(x, y, z, t) are to be discarded. Thus, an influence matrix Q_{OUT-PHYS} by a coefficient vector [symbol] (t) to the physical outside magnetic field model \vec{B_{OUT-P-MOD}}(x, y, z, t) at the N number of magnetometers 26 can be generated, and an influence matrix Q_{OUT-NP} by a coefficient vector [symbol] (t) to the physically impossible magnetic field model \vec{B_{OUT-NP-MOD}}(x, y, z, t) at the N number of magnetometers 26 can be generated.

The influence matrix Q_{OUT-P} has a size (N×p_{OUT-P}), where p_{OUT-P} is the number of modes in the physically possible magnetic field model \vec{B_{OUT-P-MOD}}(x, y, z, t) (in this case, p_{OUT-P}=8). The influence matrix Q_{OUT-NP} has a size (N×p_{OUT-NP}), where p_{OUT-NP} is the number of modes in the physically impossible magnetic field model \vec{B_{OUT-NP-MOD}}(x, y, z, t) (in this case, p_{OUT-NP}=4). The influence matrices Q_{OUT-P} and Q_{OUT-NP} may be concatenated into an influence matrix Z_{OUT} from a coefficient vector [symbol] (t) containing twelve coefficients [γ₁(t), γ₂(t), . . . γ₁₂ (t)] (i.e., p=12) to the outside magnetic field \vec{B_{OUT}}(x, y, z, t) at the N number of magnetometers 26. In this case, the influence matrix Z_{OUT} may take the form of:

Z_{OUT}=[Q_{OUT-P} Q′_{OUT-NP}], [21]

where the (p−4) leftmost column vectors of the influence matrix Z are the column vectors of the influence matrix Q_{OUT-P} that respectively correspond to the modes of the generic outside magnetic field model \vec{B_{OUT-MOD}} (x, y, z, t) to be retained (i.e., the modes of the physically possible magnetic field model \vec{B_{OUT-P-MOD}}(x, y, z, t)); and 4 rightmost column vectors of the influence matrix Z_{OUT} are the column vectors of the influence matrix Q′_{OUT-NP} that respectively correspond to the modes of the generic outside magnetic field model \vec{B_{OUT-MOD}}(x, y, z, t) to be discarded (i.e., the modes of the physically impossible magnetic field model \vec{B_{OUT-NP-MOD}}(x, y, z, t)).

The generic outside magnetic field model \vec{B_{OUT-MOD}}(x, y, z, t) may then be parameterized to generate a parameterized model of the outside magnetic field \vec{B_{OUT-PAR}}(x, y, z) by determining the least squares estimate of the coefficient vector \vec{γ*}(t) in accordance with modified equations [20a] and [20b] as follows:

\vec{γ_{P}*}(t)=[Z^{T}Z]⁻¹Z^{T}(\vec{B_{TOT-MEAS}}(x,y,z,t)−R* [symbol] (t)){1:p_{P}}; and [22a]

\vec{γ_{NP}*}(t)=[Z^{T}Z]⁻¹Z^{T}(\vec{B_{TOT-MEAS}}(x,y,z,t)−R* [symbol] (t)){p_{P}+1: p_{NP}}, [22b]

where \vec{B_{TOT-MEAS}}(x, y, z, t), Z, R, [symbol] (t), p_{P}, and p_{NP} have been defined above; the notation X{A:B} means take the Ath through Bth elements of X; \vec{γ_{P}*}(t) is the least squares solution of the coefficient vector corresponding to the influence matrix Q_{P} respectively corresponding to the modes of the physically possible magnetic field model \vec{B_{P-MOD}}(x, y, z, t); and \vec{γ_{NP}*}(t) is the least squares solution of the coefficient vector corresponding to the influence matrix Q′_{NP} respectively corresponding to the modes of the physically impossible magnetic field model \vec{B_{NP-MOD}}(x, y, z, t).

The processor 30 may then estimate the physical outside magnetic field \vec{B_{OUT-P-EST}}(x, y, z, t) at the fine magnetometers 26b by substituting the (x,y,z) locations of the fine magnetometers 26b into the basis functions of the parameterized outside magnetic field model \vec{B_{OUT-PAR}}(x, y, z, t) corresponding to the modes of the physical outside magnetic field \vec{B_{OUT-P}}(x, y, z, t); i.e., the physical outside magnetic field estimates \vec{B_{OUT-P-EST}}(x, y, z, t) at the fine magnetometers 26b may be recovered from the product of the influence matrix Z_{OUT} and the least squares fit values of the coefficient vector \vec{γ_{P}*}(t) corresponding to the modes of the physical outside magnetic field \vec{B_{OUT-P}}(x, y, z, t). The processor 30 may then use the physical outside magnetic field estimates \vec{B_{OUT-P-EST}}(x, y, z, t) at the fine magnetometers 26b to control the set of magnetic field actuators 28 to at least partially cancel the outside magnetic field \vec{B_{OUT}}(x, y, z, t), thereby suppressing the total residual magnetic field \vec{B_{TOT}} (x, y, z, t) to the baseline level at the fine magnetometers 26b. The non-physical outside magnetic field \vec{B_{OUT-P}}(x, y, z, t) at the fine magnetometers 26b may simply be ignored, and therefore, not estimated at the fine magnetometers 26b.

In another specific embodiment, instead of distinguishing between a physical outside magnetic field \vec{B_{OUT-P}}(x, y, z, t) component and a non-physical outside magnetic field \vec{B_{OUT-NP}}(x, y, z, t) component of the total residual magnetic field measurements \vec{B_{TOT}}(x, y, z, t) acquired from the magnetometers 26, the processor 30 may employ the equations [19] and [20a]-[20c] to distinguish the MEG magnetic field B_{MEG} component (i.e., the portion represented by the oval 60 in FIG. 5) and the outside magnetic field B_{OUT} component (represented by the space in the rectangle 62, but outside the oval 60) of the measured total residual magnetic field B_{TOT-MEAS} acquired from the magnetometers 26.

In particular, while MEG magnetic field B_{MEG} was ignored in equation [13], the outside magnetic field \vec{B_{OUT}}(x, y, z, t) in equation [13] can be replaced with the environmental magnetic field \vec{B_{ENV}}(x, y, z, t), which includes the outside magnetic field \vec{B_{OUT}}(x, y, z, t) and a MEG magnetic field model \vec{B_{MEG-MOD}}(x, y, z, t). Thus, a generic environmental magnetic field model \vec{B_{ENV-MOD}}(x, y, z, t) may be defined and partitioned into a MEG magnetic field model \vec{B_{MEG-MOD}}(x, y, z, t) and an outside magnetic field model \vec{B_{OUT-MOD}}(x, y, z, t), where the modes of the MEG magnetic field model \vec{B_{MEG-MOD}}(x, y, z, t) are to be retained, and the modes of the generic outside magnetic field model \vec{B_{OUT-MOD}}(x, y, z, t) are to be discarded.

A matrix of influence Q_{MEG} by a coefficient vector \vec{γ_{MEG}}(t) to the MEG magnetic field model \vec{B_{MEG-MOD}}(x, y, z, t) at the N number of magnetometers 26 can be generated. The matrix of influence Q_{MEG} may be generated using mathematical or numerical modeling (e.g., by simulating the MEG magnetic field B_{MEG} emanating from a brain to different spatial locations, e.g., at the magnetometers 26) or by the performance of calibration measurements ahead of time (i.e., measure the actual MEG magnetic field B_{MEG} emanating from a brain at different spatial locations, e.g., at the magnetometers 26).

Similarly, another matrix of influence Q_{OUT} by a coefficient vector \vec{γ_{OUT}}(t) to the generic outside magnetic field model \vec{B_{OUT-MOD}}(x, y, z, t) at the N number of magnetometers 26, can be generated. The matrix of influence Q_{OUT} may be generated using mathematical or numerical modeling (e.g., by simulating the outside magnetic field B_{OUT} at different spatial locations, e.g., at the magnetometers 26) or by the performance of calibration measurements ahead of time (i.e., measure the actual outside magnetic field B_{OUT} at different spatial locations, e.g., at the magnetometers 26).

The influence matrices Q_{MEG} and Q_{OUT} may be generated using a variety of matrix factorization methods, including SVD, the QR, LU, Jordan and other eigenvalue-based decompositions, gradient descent optimization, nonnegative matrix factorization and other types of matrix factorization, and similar methods known to a persons of ordinary skill in the art of signal processing, systemic identification, optimization, control theory, or neuroscience.

The influence matrix Q_{MEG} has a size (N×p_{MEG}), where p_{MEG} is the number of modes in the MEG magnetic field model \vec{B_{MEG-MOD}}(x, y, z, t). The influence matrix Q_{OUT} has a size (N×p_{OUT}), where p_{OUT} is the number of modes in the generic outside magnetic field model \vec{B_{OUT-MOD}}(x, y, z, t). The influence matrices Q_{MEG} and Q_{OUT} may be concatenated into an influence matrix Z from a coefficient vector [symbol] (t) to the environmental magnetic field \vec{B_{ENV}}(x, y, z, t) at the N number of magnetometers 26. In this case, the influence matrix Z may take the form of:

Z=[Q_{MEG} Q′_{OUT}], [23]

where the column vectors of the influence matrix Q_{MEG} respectively correspond to the modes of the environmental magnetic field model \vec{B_{MEG-ENV}}(x, y, z, t) to be retained (i.e., the modes of the MEG magnetic field model \vec{B_{MEG-MOD}}(x, y, z, t)); and the column vectors of the influence matrix Q′_{OUT} respectively correspond to the modes of the environmental magnetic field model \vec{B_{MEG-ENV}}(x, y, z, t) to be discarded (i.e., the modes of the generic outside magnetic field model \vec{B_{OUT-MOD}}(x, y, z, t)).

The generic environmental magnetic field model \vec{B_{ENV-MOD}}(x, y, z, t) may then be parameterized to generate a parameterized model of the environmental magnetic field \vec{B_{ENV-PAR}}(x, y, z) by determining the least squares estimate of the coefficient vector \vec{γ*}(t) in accordance with modified equations [20a] and [20b] as follows:

\vec{γ_{MEG}*}(t)=[Z^{T}Z]⁻¹Z^{T}(\vec{B_{TOT-MEAS}}(x,y,z,t)−R* [symbol] (t)){:p_{MEG}}; and [24a]

\vec{γ_{OUT}*}(t)=[Z^{T}Z]⁻¹Z^{T}(\vec{B_{TOT-MEAS}}(x,y,z,t)−R* [symbol] (t)){p_{MEG}+1:p_{OUT}}, [24b]

where \vec{B_{TOT-MEAS}}(x, y, z, t), Z, R, [symbol] (t), p_{MEG}, and p_{OUT} have been defined above; the notation X{A:B} means take the Ath through Bth elements of X; \vec{γ_{MEG}*}(t) is the least squares solution of the coefficient vector corresponding to the influence matrix Q_{MEG} respectively corresponding to the modes of the MEG magnetic field model \vec{B_{MEG-MOD}}(x, y, z, t); and \vec{γ_{OUT}*}(t) is the least squares solution of the coefficient vector corresponding to the influence matrix Q_{OUT} respectively corresponding to the modes of the generic outside magnetic field model \vec{B_{OUT-MOD}}(x, y, z, t).

The processor 30 may then estimate the MEG magnetic field \vec{B_{MEG-EST}}(x, y, z, t) at the fine magnetometers 26b by substituting the (x,y,z) locations of the fine magnetometers 26b into the basis functions of the parameterized environmental magnetic field model \vec{B_{ENV-PAR}}(x, y, z, t) corresponding to the modes of the MEG magnetic field \vec{B_{MEG}}(x, y, z, t); i.e., the MEG magnetic field estimates \vec{B_{MEG-EST}}(x, y, z, t) at the fine magnetometers 26b may be recovered from the product of the influence matrix Z and the least squares fit values of the coefficient vector \vec{γ*}(t) corresponding to the modes of the MEG magnetic field \vec{B_{MEG}}(x, y, z, t). The processor 30 may then derive the MEG signals S_{MEG} from the MEG magnetic field estimates \vec{B_{MEG-EST}}(x, y, z, t) at the fine magnetometers 26b.

The outside magnetic field \vec{B_{OUT}}(x, y, z, t) component of the environmental magnetic field estimates \vec{B_{ENV-EST}}(x, y, z, t) may simply be ignored, and therefore, not estimated at the fine magnetometers 26b. Alternatively, the processor 30 may estimate the outside magnetic field \vec{B_{OUT-EST}}(x, y, z, t) at the fine magnetometers 26b by substituting the (x,y,z) locations of the fine magnetometers 26b into the basis functions of the parameterized environmental magnetic field model \vec{B_{ENV-PAR}}(x, y, z, t) corresponding to the modes of the outside magnetic field \vec{B_{OUT}}(x, y, z, t); i.e., the outside magnetic field estimates \vec{B_{OUT-EST}}(x, y, z, t) at the fine magnetometers 26b may be recovered from the product of the influence matrix Z and the least squares fit values of the coefficient vector \vec{γ*}(t) corresponding to the modes of the outside magnetic field \vec{B_{OUT-EST}}(x, y, z, t). The processor 30 may then use the outside magnetic field estimates \vec{B_{OUT-EST}}(x, y, z, t) to control the set of magnetic field actuators 28 to at least partially cancel the outside magnetic field \vec{B_{OUT}}(x, y, z, t), thereby suppressing the total residual magnetic field \vec{B_{TOT}}(x, y, z, t) to the baseline level at the fine magnetometers 26b.

In another specific embodiment, instead of distinguishing between the MEG magnetic field B_{MEG} component and the outside magnetic field B_{OUT} component of the total residual magnetic field measurements \vec{B_{TOT}}(x, y, z, t) acquired from the magnetometers 26, the processor 30 may employ the equations [19] and [20a]-[20c] to further distinguish between a MEG magnetic field \vec{B_{MEG-OI}}(x, y, z, t) component of interest and a MEG magnetic field \vec{B_{MEG-NOI}}(x, y, z, t) component not of interest of the measured total residual magnetic field B_{TOT-MEAS} acquired from the magnetometers 26. For example, a portion of the MEG magnetic field \vec{B_{MEG}}(x, y, z, t) that is generated by neural activity in the right temporal lobe of the brain 14 may be of interest, whereas the remaining portion of the MEG magnetic field \vec{B_{MEG}}(x, y, z, t) that is generated by neural activity in other regions of the brain 14 may not be of interest.

Thus, a generic environmental magnetic field model \vec{B_{ENV-MOD}}(x, y, z, t) may be defined and partitioned into an outside magnetic field model \vec{B_{OUT-MOD}}(x, y, z, t) and a MEG magnetic field model \vec{B_{MEG-MOD}}(x, y, z, t), which may be further partitioned into a MEG magnetic field model of interest \vec{B_{MEG-OI-MOD}}(x, y, z, t) and a MEG magnetic field model not of interest B_{MEG-NOI-MOD}(x, y, z, t), where the modes of the MEG magnetic field model of interest \vec{B_{MEG-OI-MOD}}(x, y, z, t) are to be retained, and the modes of the generic outside magnetic field model \vec{B_{OUT-MOD}}(x, y, z, t) and the MEG magnetic field model not of interest B_{MEG-NOI-MOD}(x, y, z, t) are to be discarded.

The matrix of influence Q_{OUT} may generated in the manner described above, whereas a matrix of influence Q_{MEG-OI} by a coefficient vector [symbol] (t) to the MEG magnetic field model of interest \vec{B_{MEG-OI-MOD}}(x, y, z, t) at the N number of magnetometers 26 can be generated, and a matrix of influence Q_{MEG-NOI} by a coefficient vector [symbol] (t) to the MEG magnetic field model of not of interest \vec{B_{MEG-NOI-MOD}}(x, y, z, t) at the N number of magnetometers 26 can be generated. The matrices of influence Q_{MEG-OI} and Q_{MEG-NOI} may be generated using mathematical or numerical modeling (e.g., by simulating the MEG magnetic field of interest B_{MEG-OI} or the MEG magnetic field not of interest B_{MEG-NOI} emanating from a brain to different spatial locations, e.g., at the magnetometers 26) or by the performance of calibration measurements ahead of time (i.e., measure the actual MEG magnetic field of interest B_{MEG-OI} or the MEG magnetic field not of interest B_{MEG-NOI} emanating from a brain at different spatial locations, e.g., at the magnetometers 26).

The influence matrices Q_{MEG-OI} and Q_{MEG-NOI} may be generated using a variety of matrix factorization methods, including SVD, the QR, LU, Jordan and other eigenvalue-based decompositions, gradient descent optimization, nonnegative matrix factorization and other types of matrix factorization, and similar methods known to a persons of ordinary skill in the art of signal processing, systemic identification, optimization, control theory, or neuroscience.

The influence matrix Q_{MEG-OI} has a size (N×p_{MEG-OI}), where p_{MEG-OI} is the number of modes in the MEG magnetic field model of interest \vec{B_{MEG-OI-MOD}}(x, y, z, t). The influence matrix Q_{MEG-NOI} has a size (N×p_{MEG-NOI}), where p_{MEG-NOI} is the number of modes in the MEG magnetic field model not of interest \vec{B_{MEG-NOI-MOD}}(x, y, z, t). The influence matrices Q_{MEG-OI}, Q_{MEG-NOI}, and Q_{OUT} may be concatenated into an influence matrix Z from a coefficient vector [symbol] (t) to the environmental magnetic field \vec{B_{ENV}}(x, y, z, t) at the N number of magnetometers 26. In this case, the influence matrix Z may take the form of:

Z=[Q_{MEG-OI} Q_{MEG-NOI} Q_{OUT}], [25]

where the column vectors of the influence matrix Q_{MEG-OI} respectively correspond to the modes of the environmental magnetic field model \vec{B_{MEG-ENV}}(x, y, z, t) to be retained (i.e., the modes of the MEG magnetic field model of interest \vec{B_{MEG-0I-MOD}}(x, y, z, t)); the column vectors of the influence matrix Q_{MEG-NOI} respectively correspond to the modes of the environmental magnetic field model \vec{B_{MEG-ENV}}(x, y, z, t) to be discarded (i.e., the modes of the MEG magnetic field model not of interest \vec{B_{MEG-NOI-MOD}}(x, y, z, t)); and column vectors of the influence matrix Q_{OUT} respectively correspond to the modes of the environmental magnetic field model \vec{B_{MEG-ENV}}(x, y, z, t) to be discarded (i.e., the modes of the generic outside magnetic field model \vec{B_{OUT-MOD}}(x, y, z, t)).

The generic environmental magnetic field model \vec{B_{ENV-MOD}}(x, y, z, t) may then be parameterized to generate a parameterized model of the magnetic field \vec{B_{OUT-PAR}}(x, y, z) by determining the least squares estimate of the coefficient vector \vec{γ*}(t) in accordance with modified equations [20a]-[20c] as follows:

\vec{γ_{MEG-OI}*}(t)=[Z^{T}Z]⁻¹Z^{T}(\vec{B_{TOT-MEAS}}(x,y,z,t)−R* [symbol] (t)){:p_{MEG-OI}}; [26a]

\vec{γ_{MEG-NOI}*}(t)=[Z^{T}Z]⁻¹Z^{T}(\vec{B_{TOT-MEAS}}(x,y,z,t)−R* [symbol] (t)){p_{MEG-OI}+1:p_{MEG-OI}+p_{MEG-NOI}}; and [26b]

\vec{γ_{OUT}*}(t)=[Z^{T}Z]⁻¹Z^{T}(\vec{B_{TOT-MEAS}}(x,y,z,t)−R* [symbol] (t)){p_{MEG-OI}+p_{MEG-NOI}+1:p_{MEG-OI}+p_{MEG-NOI}+p_{OUT}}, [26c]

where \vec{B_{TOT-MEAS}}(x, y, z, t), Z, R, [symbol] (t), p_{MEG-OI}, p_{MEG-NOI}, and p_{OUT} have been defined above; the notation X{A:B} means take the Ath through Bth elements of X; \vec{γ_{MEG-OI}*}(t) is the least squares solution of the coefficient vector corresponding to the influence matrix Q_{MEG-GI} respectively corresponding to the modes of the MEG magnetic field model \vec{B_{MEG-OI-MOD}}(x, y, z, t); \vec{γ_{MEG-NOI}*}(t) is the least squares solution of the coefficient vector corresponding to the influence matrix Q_{MEG-NOI} respectively corresponding to the modes of the MEG magnetic field model not of interest \vec{B_{MEG-NOI-MOD}}(x, y, z, t).

The processor 30 may then estimate the MEG magnetic field of interest \vec{B_{MEG-OI-EST}}(x, y, z, t) at the fine magnetometers 26b by substituting the (x,y,z) locations of the fine magnetometers 26b into the basis functions of the parameterized environmental magnetic field model \vec{B_{ENV-PAR}}(x, y, z, t) corresponding to the modes of the MEG magnetic field of interest \vec{B_{MEG-OI}}(x, y, z, t); i.e., the MEG magnetic field of interest estimates \vec{B_{MEG-OI-EST}}(x, y, z, t) at the fine magnetometers 26b may be recovered from the product of the influence matrix Z and the least squares fit values of the coefficient vector \vec{γ*}(t) corresponding to the to the modes of the MEG magnetic field of interest \vec{B_{MEG-OI}}(x, y, z, t). The processor 30 may then derive the MEG signals S_{MEG} from the MEG magnetic field of interest estimates \vec{B_{MEG-OI-EST}}(x, y, z, t) at the fine magnetometers 26b.

The outside magnetic field \vec{B_{NON-PHYSICAL}}(x, y, z, t) and MEG magnetic field not of interest \vec{B_{MEG-NOI}}(x, y, z, t) at the fine magnetometers 26b may simply be ignored, and therefore, not estimated at the fine magnetometers 26b. Alternatively, the processor 30 may estimate the outside magnetic field \vec{B_{OUT-EST}}(x, y, z, t) at the fine magnetometers 26b by substituting the (x,y,z) locations of the fine magnetometers 26b into the basis functions of the parameterized environmental magnetic field model \vec{B_{ENV-PAR}}(x, y, z, t) corresponding to the modes of the outside magnetic field \vec{B_{OUT}}(x, y, z, t); i.e., the outside magnetic field estimates \vec{B_{OUT-EST}}(x, y, z, t) at the fine magnetometers 26b may be recovered from the product of the influence matrix Z and the least squares fit values of the coefficient vector \vec{γ*}(t) corresponding to the modes of the outside magnetic field \vec{B_{OUT-EST}}(x, y, z, t). The processor 30 may then use the outside magnetic field estimates \vec{B_{OUT-EST}}(x, y, z, t) to control the set of magnetic field actuators 28 to at least partially cancel the outside magnetic field \vec{B_{OUT}}(x, y, z, t), thereby suppressing the total residual magnetic field \vec{B_{TOT}}(x, y, z, t) to the baseline level at the fine magnetometers 26b.

Referring back to FIG. 5, the processor 30 is further configured for distinguishing the portion of the measured total residual magnetic field B_{TOT-MEAS} that corresponds to magnetic fields that are generated from electrical sources (represented by the space in the parallelogram 70) and the portion of the measured total residual magnetic field B_{TOT-MEAS} that corresponds to magnetic fields that are generated from permanent magnets (represented by the space in the rectangle 62, but outside the parallelogram 70). Because the MEG magnetic field B_{MEG} is generated from electrical current associated with neural activity in the brain 12, whereas the Earth's magnetic field is generated from a permanent magnet (i.e., the Earth's iron core), in effect, the MEG magnetic field B_{MEG} represented in the measured total residual magnetic field B_{TOT-MEAS} and the Earth's magnetic field (as a portion of the outside magnetic field B_{OUT}) is distinguished, as represented by the union space 72 between the oval 60 and the parallelogram 70.

In particular, the electromagnetic nature of magnetic fields that are generated from electrical sources are different from the electromagnetic nature of magnetic fields that are generated from permanent magnets for a variety of reasons. For example, permanent magnets have a persistent magnetization, and thus, the processor 30 may reduce the content of the outside magnetic field B_{OUT} in the measured total residual magnetic field B_{TOT-MEAS} by eliminating the content of the measured total residual magnetic field B_{TOT-MEAS} corresponding to persistent magnetization. Furthermore, the electrical current along a neural connection that is primarily axial in nature may be distinguishable from a closed electrical current loop, which is more similar to that from a permanent magnet, and thus, the processor 30 may reduce the content of the outside magnetic field B_{OUT} in the measured total residual magnetic field B_{TOT-MEAS} by eliminating the content of the measured total residual magnetic field B_{TOT-MEAS} corresponding to closed electrical current loops. In some cases, there may be closed electrical loops in the brain. However, the scaling of magnetic field delay differs from electrical current in the brain than permanent magnets. Thus, the processor 30 may reduce the content of the outside magnetic field B_{OUT} in the measured total residual magnetic field B_{TOT-MEAS} by eliminating the content of the measured total residual magnetic field B_{TOT-MEAS} corresponding to magnetic fields that decay in space with a scale of that from the permanent magnets.

Thus, referring back to FIG. 5, the combination of these magnetic field distinguishing techniques may yield the true MEG magnetic field B_{MEG-TRUE} from the measured total residual magnetic field B_{TOT-MEAS}, as represented by the union space 74 between the oval 60, bottom triangle 64, and parallelogram 70.

The processor 30 may be configured for performing the magnetic field distinguishing techniques in any suitable order. Furthermore, the magnetic field distinguishing techniques can be combined as “AND” logic or “OR” logic. For example, is there are conditions A, B, and C that can respectively be associated with the magnetic field distinguishing techniques. Then the processor 30 may accept the portion of the total residual magnetic field B_{TOT} identified as the MEG magnetic field B_{MEG} only if the conditions A-C (or any combination of conditions A-C) are satisfied or may accept the portion of the total residual magnetic field B_{TOT} identified as the MEG magnetic field B_{MEG} if one of the conditions A-C is satisfied. Furthermore, one condition for a magnetic field distinguishing technique may be dynamically varied based on the satisfaction of the satisfaction of the condition of another one of the magnetic field distinguishing techniques. For example, if the condition A for one of the magnetic field distinguishing techniques is satisfied, then the threshold for satisfying the condition B for another one of the magnetic field distinguishing techniques may be lowered.

Thus, it can be appreciated from the foregoing that the signal acquisition unit 18 eliminates large portions of the total residual magnetic field B_{TOT} that do not correspond to the true MEG magnetic field B_{MEG-TRUE} by cleverly combining various signal discriminating techniques, and in particular, based on Maxwell's equations, temporal frequency, spatial frequency, and amplitude.

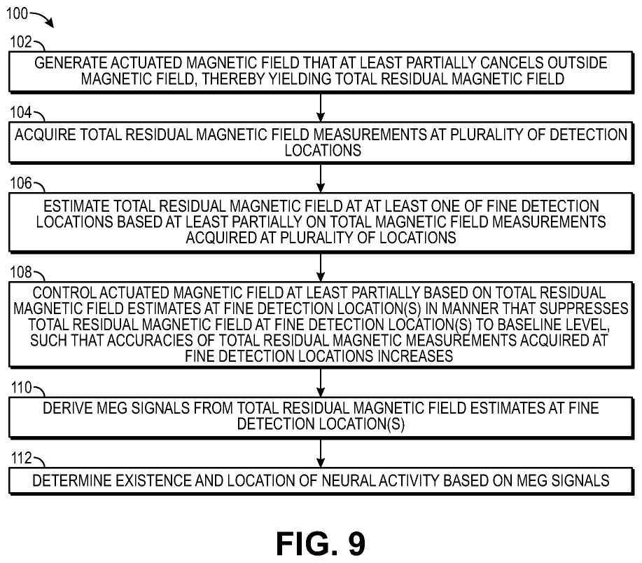

Referring now to FIG. 9, one exemplary method 100 of identifying and localizing neural activity in the brain 14 of a user 12 will be described.

The method 100 comprises generating the actuated magnetic field B_{ACT} that at least partially cancels an outside magnetic field B_{OUT} (e.g., via the set of magnetic field actuators 28 of the signal acquisition unit 18), thereby yielding a total residual magnetic field B_{TOT} (step 102). In the preferred embodiment, the actuated magnetic field B_{ACT} is generated in all three dimensions and is uniform, although in alternative embodiments, the actuated magnetic field B_{ACT} may be generated in less three dimensions and may be non-uniform (e.g., a gradient).

The method 100 further comprises acquiring the total residual magnetic field measurements B_{TOT-MEAS} respectively at a plurality of detection locations (e.g., from the coarse magnetometers 26a and/or fine magnetometers 26b of the signal acquisition unit 18) (step 104). The method 100 further comprises estimating the total residual magnetic field B_{TOT-MEAS} at at least one the fine detection locations (e.g., at the fine magnetometers 26b of the signal acquisition unit 18) based at least partially on the total residual magnetic field measurements B_{TOT-MEAS} respectively acquired from the detection locations (step 106).

The method 100 further comprises controlling the actuated magnetic field B_{ACT} at least partially based on the total residual magnetic field estimates B_{TOT-EST} at the fine detection location(s) in a manner that suppresses the total residual magnetic field B_{TOT} at the fine detection location(s) to a baseline level (by cancelling the outside magnetic field B_{OUT}, e.g., via the coarse feedback control loop 50 and/or fine feedback control loop 52 and sending noise-cancelling control signals C to the set of magnetic field actuators 28 of the signal acquisition unit 18), such that accuracies of the total residual magnetic field measurements B_{TOT-MEAS} acquired at the fine detection location(s) increase (e.g., fine magnetometers 26b of the signal acquisition unit 18 come in-range) (step 108).

In particular, the total residual magnetic field B_{TOT} is suppressed at the fine detection location(s) (e.g., at the fine magnetometers 26b of the signal acquisition unit 18) to the baseline level at the fine detection location(s) by cancelling the outside magnetic field B_{OUT} component relative to the MEG magnetic field B_{MEG} component of the total residual magnetic field measurements B_{TOT-MEAS} acquired from the fine detection location(s) based on a combination of the temporal frequency of the outside magnetic field B_{OUT} (e.g., by suppressing the total residual magnetic field measurements B_{TOT-MEAS} acquired from the fine detection location(s) at DC and harmonic temporal frequencies), the spatial frequency of the outside magnetic field B_{OUT} (e.g., by suppressing the total residual magnetic field measurements B_{TOT-MEAS} acquired from the fine detection location(s) at relatively low spatial frequencies) and/or a strength of the outside magnetic field B_{OUT} (e.g., by suppressing the total residual magnetic field measurements B_{TOT-MEAS} acquired from the fine detection location(s) at relatively high strength frequency components).

Although the outside magnetic field B_{OUT} is at least partially cancelled at the fine detection location(s) by the actuated magnetic field B_{ACT} at selected temporal frequencies, spatial frequencies, and/or strengths as a means of suppressing the total residual magnetic field B_{TOT} at the fine detection location(s) to the baseline level, in alternative embodiments, the outside magnetic field B_{OUT} component of the total residual magnetic field measurements B_{TOT-MEAS} acquired from the fine detection location(s) may be suppressed external to the feedback control loop during a post-processing step, in which case, the total residual magnetic field B_{TOT} at the fine detection location(s) may be suppressed to the baseline level utilizing other techniques.

The method further comprises deriving a plurality of MEG signals S_{MEG} respectively from the total residual magnetic field estimates B_{TOT-EST} acquired from the fine detection location(s) (e.g., via the signal acquisition unit 18) (step 110). That is, because the total residual magnetic field B_{TOT} at the fine detection location(s) contains the MEG magnetic field B_{MEG} from the brain 14 of the user 12, and thus by inference, the total residual magnetic field estimates B_{TOT-EST} at the fine detection location(s) contains estimates of the MEG magnetic field B_{MEG} from the brain 14 of the user 12, the MEG signals S_{MEG} can be extracted from the total residual magnetic field estimates B_{TOT-EST} at the fine detection location(s). The method 100 lastly comprises determining the existence and detection location of neural activity in the brain 14 of the user 12 based on the MEG signals S_{MEG} (e.g., via the signal processing unit 20) (step 112).

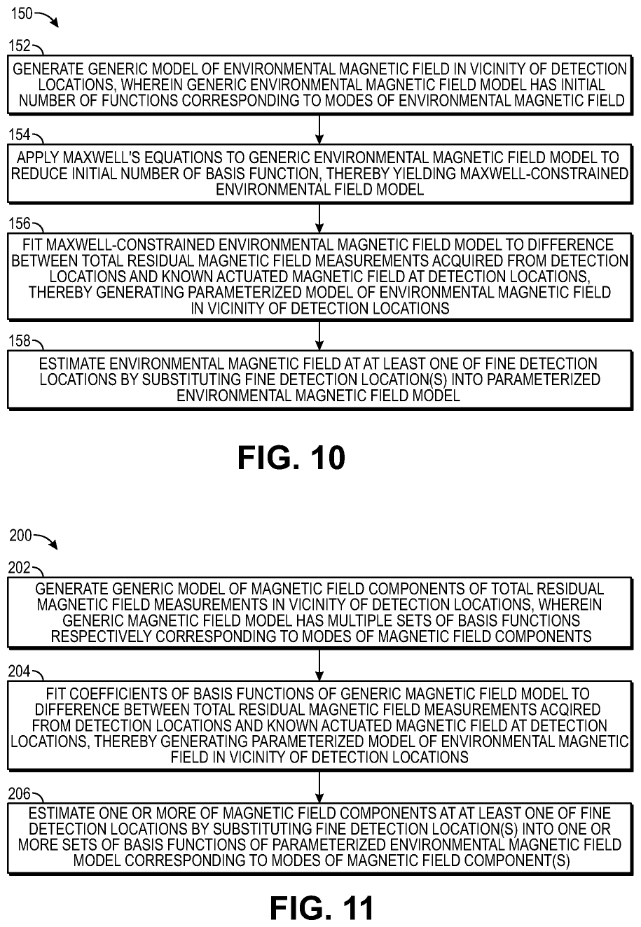

Referring now to FIG. 10, one method 150 of estimating the environmental magnetic field B_{ENV-EST} at the fine detection location(s) (e.g., at the fine magnetometers 26b of the signal acquisition unit 18) in a manner that removes, or at least reduces, the non-physical portion (i.e., the physically impossible portion) of a magnetic field component of the environmental magnetic field B_{ENV} (in this case, the components of the outside magnetic field B_{OUT} and the MEG magnetic field B_{MEG}) from total residual magnetic field measurements B_{TOT-MEAS} acquired from the plurality of detection locations (e.g., from the coarse magnetometers 26a and/or fine magnetometers 26b of the signal acquisition unit 18), thereby reducing errors in the total residual magnetic field B_{TOT} measurements. It should be appreciated that the method 150 may be generalized to remove or at least reduce the non-physical portion of any magnetic field component from a measured arbitrary magnetic field.

The method 150 comprises generating a generic model of the environmental magnetic field B_{ENV-MOD} in the vicinity of the detection locations, the generic model comprising an initial number of basis functions corresponding to the modes of the environmental magnetic field B_{ENV-MOD} (step 152). In one embodiment, the generic model B_{ENV-MOD} comprises basis functions for both the outside magnetic field B_{OUT} and the MEG magnetic field B_{MEG}, such that the non-physical portion of the components of both the outside magnetic field B_{OUT} and the MEG magnetic field B_{MEG} can be suppressed in the total residual magnetic field measurements B_{TOT-MEAS} acquired from the detection locations, although in alternative embodiments, the generic model comprises basis functions for only the outside magnetic field B_{OUT} or only the MEG magnetic field B_{MEG}, such that the non-physical portion of the components of either the outside magnetic field B_{OUT} or the MEG magnetic field B_{MEG} can be suppressed in the total residual magnetic field measurements B_{TOT-MEAS} acquired at the detection locations.

The method 150 further comprises applying Maxwell's equations to the environmental magnetic field model B_{ENV-MOD} to reduce the initial number of different basis functions, thereby yielding a Maxwell-constrained model of the environmental magnetic field B_{ENV-MAXWELL} (step 154). IN the case where the generic environmental magnetic field model B_{ENV-MOD} comprises basis functions corresponding to modes of the outside magnetic field B_{OUT}, such basis functions may comprise 0^{th} order basis functions and 1st order basis functions. In another embodiment, the basis functions comprise at least one non-linear basis function (e.g., a vector spherical harmonics (VSH) basis function).

The method 150 further comprises parameterizing the Maxwell-constrained environmental magnetic field model B_{ENV-MAXWELL} at least partially based on the total residual magnetic field measurements B_{TOT-MEAS} acquired from the detection locations, and in the illustrated embodiment, based on the total residual magnetic field measurements B_{TOT-MEAS} acquired at the detection locations and the known actuated magnetic field B_{ACT-KNOWN} at the detection locations, thereby yielding a parameterized environmental magnetic field model B_{ENV-PAR}. In the illustrated embodiment, the Maxwell-constrained environmental magnetic field model B_{ENV-MAXWELL} is parameterized by fitting the Maxwell-constrained environmental magnetic field model B_{ENV-MAXWELL} to a difference between the total residual magnetic field measurements B_{TOT-MEAS} acquired at the detection locations and the known actuated magnetic field B_{ACT-KNOWN} at the detection locations (e.g., using a least squares optimization technique) (step 156).

For example, the coefficients of the basis functions in Maxwell-constrained environmental magnetic field model B_{ENV-MAXWELL} may be fitted to the difference between the total residual magnetic field measurements B_{TOT-MEAS} acquired at the detection locations and the known actuated magnetic field B_{ACT-KNOWN} at the detection locations, e.g., using a least squares optimization technique. The fitted coefficients may then be incorporated into the Maxwell-constrained environmental magnetic field model B_{ENV-MAXWELL}, thereby yielding the parameterized environmental magnetic field model B_{ENV-PAR}.

The method 150 lastly comprises estimating the environmental magnetic field B_{ENV-EST} at at least one the fine detection locations (e.g., at the fine magnetometers 26b of the signal acquisition unit 18) based on the parameterized environmental magnetic field model B_{ENV-PAR}, and in particular, by substituting the fine detection location(s) into the parameterized environmental magnetic field model B_{ENV-PAR} (step 158). It should be appreciated that, due to the previous application of Maxwell's equations to the generic environmental magnetic model B_{ENV-MOD}, the non-physical portion of the estimated environmental magnetic field model B_{ENV-EST} of the measured at the fine detection location(s) is less than the non-physical portion of the environmental magnetic field model B_{ENV} component of the total residual magnetic field measurements B_{TOT-MEAS} acquired at the fine detection location(s).

It should be appreciated that, because the non-physical portion of the environmental magnetic field B_{ENV} component of the measurements B_{TOT-MEAS} acquired from the location(s) has been reduced by using Maxwell's equations to provide more accurate total residual magnetic field estimates B_{TOT-EST}, the actuated magnetic field B_{ACT}, the control of which is at least partially based on the total residual magnetic field estimates B_{TOT-EST} at the fine detection location(s) in the method 100 described above, more accurately cancels the outside magnetic field B_{OUT} at the fine detection location(s), and thus more effectively suppresses the total residual magnetic field B_{TOT} at the fine detection location(s) to the baseline level, such that accuracies of the total residual magnetic field measurements B_{TOT-MEAS} acquired at the fine detection location(s) increase.

Notably, in the case where the Maxwell's equations have been applied to the environmental magnetic field B_{ENV} component of the total residual magnetic field measurements B_{TOT-MEAS} acquired from the fine detection location(s) in a manner that reduces the non-physical portion of the MEG magnetic field B_{MEG} component of the total residual magnetic field measurements B_{TOT-MEAS} acquired from the fine detection location(s), the accuracy of the MEG signals S_{MEG} extracted from the total residual magnetic field estimates B_{TOT-EST} at the fine detection location(s) will be increased.

Referring now to FIG. 11, one method 200 of estimating at least one magnetic field component of the total residual magnetic field measurements B_{TOT-MEAS} at the fine detection location(s) (e.g., at the fine magnetometers 26b of the signal acquisition unit 18) will now be described. It should be appreciated that the method 200 may be generalized to estimate any magnetic field component of any measured arbitrary magnetic field.

The method 200 comprises generating a generic model of a plurality of magnetic field components of the total residual magnetic field measurements B_{TOT-MEAS} in the vicinity of the detection locations, wherein the generic magnetic field model comprises a plurality of basis functions having multiple sets of basis functions respectively corresponding to modes of the magnetic field components (step 202). In one embodiment, the generic magnetic field model B_{MOD} comprises a coefficient vector [symbol] and a matrix of influence Z from the coefficient vector [symbol] to the magnetic field components of the total residual magnetic field B_{TOT}. The coefficient vector [symbol] has a p number of coefficients respectively corresponding to the basis functions, the influence matrix Z comprises a p number of column vectors and an N number of row vectors respectively corresponding to the total residual magnetic field measurements B_{TOT-MEAS} acquired from the detection locations, and p is less than N.

The method 200 further comprises parameterizing the generic magnetic field model B_{MOD} by simultaneously fitting coefficients of the basis functions of the generic magnetic field model B_{MOD} at least partially to the total residual magnetic field measurements B_{TOT-MEAS} acquired from the detection locations, thereby yielding a parameterized model of the magnetic field components B_{PAR} of the total residual magnetic field B_{TOT} in the vicinity of the detection locations. In the illustrated embodiment, the generic magnetic field model B_{MOD} is parameterized by simultaneously fitting the coefficients of the basis functions at least partially to a difference between the total residual magnetic field measurements B_{TOT-MEAS} acquired at the detection locations and the known actuated magnetic field B_{ACT-KNOWN} at the detection locations (e.g., using a least squares optimization technique) (step 204). The coefficients of the plurality of basis functions may be simultaneously fitted at least partially to the total residual magnetic field measurements B_{TOT-MEAS} acquired from the detection locations by equating the product of the coefficient vector [symbol] and the influence matrix Z to the total residual magnetic field measurements B_{TOT-MEAS} acquired from the detection locations and simultaneously fitting the p number of coefficients in the coefficient vector [symbol] at least partially to the difference between the total residual magnetic field measurements B_{TOT-MEAS} acquired at the detection locations and the known actuated magnetic field B_{ACT-KNOWN} at the detection locations.

The method 200 lastly comprises estimating one or more of the magnetic field components of the total residual magnetic field measurement B_{TOT-MEAS} at each of at least one of the fine detection locations (e.g., from one of the fine magnetometers 26b of the signal acquisition unit 18) respectively based on the multiple sets of basis functions of the parameterized magnetic field model B_{PAR} and in particular, by substituting the fine detection location(s) into the set(s) of basis functions corresponding to the modes of the one or more magnetic field components (step 206). That is, a first one of the magnetic field components of the total residual magnetic field measurement B_{TOT-MEAS} can be estimated at each of at least one of the fine detection locations (e.g., from one of the fine magnetometers 26b of the signal acquisition unit 18) respectively based on a first set of the basis functions of the parameterized magnetic field model B_{PAR} (e.g., by substituting the fine detection locations(s) into the set of basis functions corresponding to the modes of the first magnetic field component); a second one of the magnetic field components of the total residual magnetic field measurement B_{TOT-MEAS} can be estimated at each of at least one of the fine detection locations (e.g., from one of the fine magnetometers 26b of the signal acquisition unit 18) respectively based on a second set of the basis functions of the parameterized magnetic field model B_{PAR} (e.g., by substituting the fine detection locations(s) into the set of basis functions corresponding to the modes of the first magnetic field component); and so on.

In one embodiment, the parameterized magnetic field model B_{PAR} is a parameterized outside magnetic field model B_{OUT-PAR}, the magnetic field components of the total residual magnetic field measurements B_{TOT-MEAS} comprise a physical outside magnetic field B_{OUT-P} component and a non-physical outside magnetic field B_{OUT-NP} of the total residual magnetic field measurements B_{TOT-MEAS}, and the first set of basis functions of the parameterized outside magnetic field model B_{OUT-PAR} corresponds to modes of the outside magnetic field B_{OUT-P} that are physically possible, while the second set of basis functions of the parameterized outside magnetic field model B_{OUT-PAR} corresponds to modes of the outside magnetic field B_{OUT-NP} that are physically impossible. In this case, the physical outside magnetic field B_{OUT-P} component of total residual magnetic field measurements B_{TOT-MEAS} can be estimated at each of the fine detection location(s) based on the first set of basis functions, while ignoring the second set of basis functions, of the parameterized outside magnetic field model B_{OUT-PAR}. The physical outside magnetic field estimates B_{OUT-P-EST} at the fine detection location(s) can then be used in the step 108 of the method 100 as a means to control the actuated magnetic field B_{ACT} to at least partially cancel the outside magnetic field B_{OUT} at the fine location(s) in a manner that suppresses the total residual magnetic field B_{TOT} at the fine detection location(s) to the baseline level.

In another embodiment, the parameterized magnetic field model B_{PAR} is a parameterized environmental magnetic field model B_{ENV-PAR}, the magnetic field components of the total residual magnetic field measurements B_{TOT-MEAS} comprise the MEG magnetic field B_{MEG} and the outside magnetic field B_{OUT}, and the first set of basis functions of the parameterized environmental magnetic field model B_{ENV-PAR} corresponds to modes in the MEG magnetic field B_{MEG}, while the second set of basis functions of the parameterized environmental magnetic field model B_{ENV-PAR} corresponds to modes in the outside magnetic field B_{OUT}.

In this case, the MEG magnetic field B_{MEG} component of the total residual magnetic field measurements B_{TOT-MEAS} can be estimated at each of the fine detection location(s) based on the first set of basis functions of the parameterized environmental magnetic field model B_{ENV-PAR}, while the outside magnetic field B_{OUT} component of the total residual magnetic field measurements B_{TOT-MEAS} can be estimated at each of the fine detection location(s) based on the second set of basis functions of the parameterized environmental magnetic field model B_{ENV-PAR}. The MEG signals S_{MEG} may be derived from the MEG magnetic field B_{MEG-EST} at the detection location(s) external to the feedback control loop in step 110 of the method 100, while the outside magnetic field estimates B_{OUT-EST} may either be ignored or used in the step 108 of the method 100 as a means to control the actuated magnetic field B_{ACT} to at least partially cancel the outside magnetic field B_{OUT} at the fine location(s) in a manner that suppresses the total residual magnetic field B_{TOT} at the fine detection location(s) to the baseline level.

In still another embodiment, the parameterized magnetic field model B_{PAR} is a parameterized environmental magnetic field model B_{ENV-PAR}, the magnetic field components of the total residual magnetic field measurements B_{TOT-MEAS} comprise a MEG magnetic field of interest B_{MEG-OI} and a MEG magnetic field of not of interest B_{MEG-NOI}, and the first set of basis functions of the generic magnetic field model B_{MOD} corresponds to modes of the MEG magnetic field B_{MEG-OI} of interest, while the second set of basis functions of the generic magnetic field model B_{MOD} corresponds to modes of the MEG magnetic field a B_{MEG-NOI} not of interest. In this case, the MEG magnetic field of interest B_{MEG-OI} component of total residual magnetic field measurement B_{TOT-MEAS} can be estimated at each of the fine detection location(s) based on the first set of basis functions of the parameterized magnetic field model B_{PAR}, while the second set of basis functions may be ignored. The MEG signals S_{MEG} may be derived from the estimates of the MEG magnetic field of interest B_{MEG-OI} at the detection location(s) external to the feedback control loop in step 110 of the method 100.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. A system, comprising:
a plurality of magnetometers configured for taking measurements of an arbitrary magnetic field having one or more magnetic field components; and
a processor configured for acquiring the arbitrary magnetic field measurements from the plurality of magnetometers, generating a generic model of at least one of the one or more magnetic field components of the arbitrary magnetic field in the vicinity of the plurality of magnetometers, wherein the generic magnetic field model comprises an initial number of different basis functions, applying Maxwell's equations to the generic magnetic field model to reduce the initial number of different basis functions, thereby yielding a Maxwell-constrained model of the at least one magnetic field component of the arbitrary magnetic field, estimating the at least one magnetic field component of the arbitrary magnetic field at each of at least one of the plurality of magnetometers based on the constrained magnetic field model and the arbitrary magnetic field measurements acquired from the each at least one magnetometer.

2. The system of claim 1, wherein the at least one magnetic field component of the magnetic field measurement acquired from the each at least one magnetometer comprises a physical portion and a non-physical portion, and the at least one magnetic field component estimate at the at least one magnetometer has a physical portion and a non-physical portion, wherein the non-physical portion of the at least one magnetic field component estimate at the each of at least one magnetometer is respectively less than the non-physical portion of the at least one magnetic field component of the magnetic field measurement acquired from the each at least one magnetometer.

3. The system of claim 1, wherein the processor is configured for estimating the at least one magnetic field component at the each of at least one magnetometer by parameterizing the constrained magnetic field model at least partially based on the arbitrary magnetic field measurements acquired from the plurality of magnetometers, thereby yielding a parameterized model of the at least one magnetic field component of the arbitrary magnetic field in the vicinity of the plurality of magnetometers, and substituting each location of the at least one magnetometer into the parameterized magnetic field model.

4. The system of claim 3, wherein the processor is configured for parameterizing the constrained magnetic field model by fitting the coefficients of the reduced number of basis functions of the constrained magnetic field model at least partially to the arbitrary magnetic field measurements acquired from the plurality of magnetometers.

5. The system of claim 4, wherein the processor is configured for fitting the coefficients of the reduced number of basis functions at least partially to the arbitrary magnetic field measurements acquired from the plurality of magnetometers using a least squares optimization technique.

6. The system of claim 4, wherein the processor is configured for parameterizing the constrained magnetic field model by incorporating the fitted coefficients into the constrained magnetic field model.

7. The system of claim 1, wherein the initial number of basis functions comprises 0^{th} order basis functions and 1st order basis functions.

8. The system of claim 1, wherein the initial number of basis functions comprises at least one non-linear basis function.

9. The system of claim 8, wherein the at least one non-linear basis function comprises a vector spherical harmonics (VSH) basis function.

10. The system of claim 1, wherein the one or more magnetic field components of the arbitrary magnetic field comprises an outside magnetic field and a magnetoencephalography (MEG) magnetic field, the at least one magnetic field component of the arbitrary magnetic field comprises the outside magnetic field, the initial number of different basis functions in the generic magnetic field model comprises basis functions for the outside magnetic field, and the at least one magnetic field component estimate at the each of at least one magnetometer comprises an outside magnetic field estimate.

11. The system of claim 10, wherein the at least one magnetic field component of the arbitrary magnetic field further comprises the MEG magnetic field, wherein the initial number of different basis functions in the generic magnetic field model further comprises basis functions for the MEG magnetic field, and the at least one magnetic field component estimate at the each of at least one magnetometer further comprises a MEG magnetic field estimate.

12. The system of claim 10,
wherein the arbitrary magnetic field is a total residual magnetic field, the system further comprising at least one magnetic field actuator configured for generating an actuated magnetic field that at least partially cancels the outside magnetic field at the each of at least one magnetometer, thereby yielding the total residual magnetic field at the each of at least one magnetometer, such that the arbitrary magnetic field measurements acquired from the plurality of magnetometers are total residual magnetic field measurements acquired from the plurality of magnetometers;
wherein the processor is configured for estimating the total residual magnetic field at the each of at least one magnetometer based on the outside magnetic field estimate at the each of at least one magnetometer and the total residual magnetic field measurements acquired from the plurality of magnetometers, and controlling the actuated magnetic field at least partially based on the total residual magnetic field estimate at the each of at least one magnetometer in a manner that suppresses the total residual magnetic field at the each of at least one magnetometer to a baseline level, such that the each at least one magnetometer is in-range.

13. The system of claim 12, wherein the processor is configured for estimating the total residual magnetic field at the each of at least one magnetometer by determining a known actuated magnetic field at the each of at least one magnetometer, and estimating the total residual magnetic field at the each of at least one magnetometer based on the known actuated magnetic field at the each of at least one magnetometer and the outside magnetic field estimate at the each of at least one magnetometer.

14. The system of claim 13, wherein the at least one magnetic field actuator respectively has at least one actuation strength, and wherein the processor is configured for determining the known actuated magnetic field at the each of at least one magnetometer based on a known profile of the at least one magnetic field actuator and the at least one actuation strength of the at least one magnetic field actuator.

15. The system of claim 12, wherein the processor is configured for estimating the total residual magnetic field at the each of at least one magnetometer by summing the known actuated magnetic field at the each of at least one magnetometer and the outside magnetic field estimate at the each of at least one magnetometer.

16. The system of claim 12, further comprising:
a signal acquisition unit configured for being worn on a head of a user, the signal acquisition unit comprising a support structure, the at least one magnetic field actuator affixed to the support structure, the plurality of magnetometers affixed to the support structure, the signal acquisition unit configured for deriving a MEG signal from the total residual magnetic field estimate at the each of at least one magnetometer; and
a signal processing unit configured for determining an existence of neural activity in the brain of the user at least partially based on the MEG signal derived from the total residual magnetic field estimate at the each of at least one magnetometer.

17. The system of claim 12, wherein the at least one magnetic field actuator comprises three orthogonal magnetic field actuators.

18. The system of claim 12, wherein each of the at least one magnetic field actuator comprises a uniform magnetic field actuator.

19. The system of claim 12, wherein the plurality of magnetometers comprises a plurality of coarse magnetometers and a plurality of fine magnetometers, and wherein the each at least one magnetometer comprises a fine magnetometer.

20. The system of claim 19, wherein each of the plurality of coarse magnetometers is a flux gate magnetometer, and the fine magnetometer is an optically pumped magnetometer (OPM).

21. A method, comprising:
acquiring measurements of an arbitrary magnetic field having one or more magnetic field components at a plurality of detection locations;
generating a generic model of at least one of the one or more magnetic field components of the arbitrary magnetic field in the vicinity of the plurality of detection locations, wherein the generic magnetic field model comprises an initial number of different basis functions;
applying Maxwell's equations to the generic magnetic field model to reduce the initial number of different basis functions, thereby yielding a Maxwell-constrained model of the at least one magnetic field component of the arbitrary magnetic field;
estimating the at least one magnetic field component of the arbitrary magnetic field at each of at least one of the plurality of detection locations based on the constrained magnetic field model and the arbitrary magnetic field measurements acquired from the each at least one detection location.

22. The method of claim 21, wherein the at least one magnetic field component of the magnetic field measurement acquired from the each at least one detection location comprises a physical portion and a non-physical portion, and the at least one magnetic field component estimate at the at least one detection location has a physical portion and a non-physical portion, wherein the non-physical portion of the at least one magnetic field component estimate at the each of at least one detection location is respectively less than the non-physical portion of the at least one magnetic field component of the magnetic field measurement acquired from the each at least one detection location.

23. The method of claim 21, wherein estimating the at least one magnetic field component at the each of at least one detection location comprises:
parameterizing the constrained magnetic field model at least partially based on the arbitrary magnetic field measurements acquired from the plurality of detection locations, thereby yielding a parameterized model of the at least one magnetic field component of the arbitrary magnetic field in the vicinity of the plurality of detection locations; and
substituting the each at least one detection location into the parameterized magnetic field model.

24. The method of claim 23, parameterizing the constrained magnetic field model comprises fitting the coefficients of the reduced number of basis functions of the constrained magnetic field model at least partially to the arbitrary magnetic field measurements acquired from the plurality of detection locations.

25. The method of claim 24, wherein the coefficients of the reduced number of basis functions are fitted to the arbitrary magnetic field measurements acquired from the plurality of detection locations using a least squares optimization technique.

26. The method of claim 24, wherein parameterizing the constrained magnetic field model comprises incorporating the fitted coefficients into the constrained magnetic field model.

27. The method of claim 21, wherein the initial number of basis functions comprises 0^{th} order basis functions and 1st order basis functions.

28. The method of claim 21, wherein the initial number of basis functions comprises at least one non-linear basis function.

29. The method of claim 28, wherein the at least one non-linear basis function comprises a vector spherical harmonics (VSH) basis function.

30. The method of claim 21, wherein the one or more magnetic field components of the arbitrary magnetic field comprises an outside magnetic field and a magnetoencephalography (MEG) magnetic field, the at least one magnetic field component of the arbitrary magnetic field comprises the outside magnetic field, the initial number of different basis functions in the generic magnetic field model comprises basis functions for the outside magnetic field, and the at least one magnetic field component estimate at the each of at least one detection location comprises an outside magnetic field estimate.

31. The method of claim 30, wherein the at least one magnetic field component of the arbitrary magnetic field further comprises the MEG magnetic field, wherein the initial number of different basis functions in the generic magnetic field model further comprises basis functions for the MEG magnetic field, and the at least one magnetic field component estimate at the each of at least one detection location further comprises an outside magnetic field estimate.

32. The method of claim 30, wherein the arbitrary magnetic field is a total residual magnetic field, the system further comprising:
generating an actuated magnetic field that at least partially cancels the outside magnetic field at the each of at least one detection location, thereby yielding the total residual magnetic field at the each of at least one detection location, such that the arbitrary magnetic field measurements acquired from the plurality of detection locations are total residual magnetic field measurements acquired from the plurality of detection locations;
estimating the total residual magnetic field at the each of at least one detection location based on the outside magnetic field estimate at the each of at least one detection location and the total residual magnetic field measurements acquired from the plurality of detection locations; and
controlling the actuated magnetic field at least partially based on the total residual magnetic field estimate at the each of at least one detection location in a manner that suppresses the total residual magnetic field at the each of at least one detection location to a baseline level, such that an accuracy of the total residual magnetic field at the each of at least one detection location increases.

33. The method of claim 32, wherein estimating the total residual magnetic field at the each of at least one detection location comprises:
determining a known actuated magnetic field at the each of at least one detection location; and
estimating the total residual magnetic field at the each of at least one detection location based on the known actuated magnetic field at the each of at least one detection location and the outside magnetic field estimate at the each of at least one detection location.

34. The method of claim 33, wherein the known actuated magnetic field is determined at the each of at least one detection location based on a known profile of the actuated magnetic field and an actuation strength of the actuated magnetic field.

35. The method of claim 32, wherein estimating the total residual magnetic field at the each of at least one detection location comprises summing the known actuated magnetic field at the each of at least one detection location and the outside magnetic field estimate at the each of at least one detection location.

36. The method of claim 32, further comprising:
deriving a MEG signal from the total residual magnetic field estimate at the each of at least one detection location; and
determining an existence of neural activity in the brain of a user at least partially based on the MEG signal derived from the total residual magnetic field estimate at the each of at least one detection location.

37. The method of claim 32, wherein the actuated magnetic field is generated in three dimensions.

38. The method of claim 32, wherein the actuated magnetic field is uniform.

39. The method of claim 38, wherein the total residual magnetic field measurements acquired from the plurality of detection locations comprises coarse total residual magnetic field measurements and fine total residual magnetic field measurements, and wherein at least one of the fine total residual magnetic field measurements is acquired from the at least one detection location.

40.-98. (canceled)
